SEQUENCE LISTING (55 sequences)

SEQ ID NO: 1 - protein, 999 aa, Artificial Sequence
  Human MerTK
MGPAPLPLLLGLFLPALWRRAITEAREEAKPYPLFPGPFPGSLQTDHTPLLSLPHASGYQPALMFSPTQPGRPHTGNVAIPQVTSVESKPLPPLAFKHTVGHIILSEHKGVKFNCSISVPNIYQDTTISWWKDGKELLGAHHAITQFYPDDEVTAIIASFSITSVQRSDNGSYICKMKINNEEIVSDPIYIEVQGLPHFTKQPESMNVTRNTAFNLTCQAVGPPEPVNIFWVQNSSRVNEQPEKSPSVLTVPGLTEMAVFSCEAHNDKGLTVSKGVQINIKAIPSPPTEVSIRNSTAHSILISWVPGFDGYSPFRNCSIQVKEADPLSNGSVMIFNTSALPHLYQIKQLQALANYSIGVSCMNEIGWSAVSPWILASTTEGAPSVAPLNVTVFLNESSDNVDIRWMKPPTKQQDGELVGYRISHVWQSAGISKELLEEVGQNGSRARISVQVHNATCTVRIAAVTRGGVGPFSDPVKIFIPAHGWVDYAPSSTPAPGNADPVLIIFGCFCGFILIGLILYISLAIRKRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 2 - protein, 505 aa, Artificial Sequence
  Human MerTK ECD
MGPAPLPLLLGLFLPALWRRAITEAREEAKPYPLFPGPFPGSLQTDHTPLLSLPHASGYQPALMFSPTQPGRPHTGNVAIPQVTSVESKPLPPLAFKHTVGHIILSEHKGVKFNCSISVPNIYQDTTISWWKDGKELLGAHHAITQFYPDDEVTAIIASFSITSVQRSDNGSYICKMKINNEEIVSDPIYIEVQGLPHFTKQPESMNVTRNTAFNLTCQAVGPPEPVNIFWVQNSSRVNEQPEKSPSVLTVPGLTEMAVFSCEAHNDKGLTVSKGVQINIKAIPSPPTEVSIRNSTAHSILISWVPGFDGYSPFRNCSIQVKEADPLSNGSVMIFNTSALPHLYQIKQLQALANYSIGVSCMNEIGWSAVSPWILASTTEGAPSVAPLNVTVFLNESSDNVDIRWMKPPTKQQDGELVGYRISHVWQSAGISKELLEEVGQNGSRARISVQVHNATCTVRIAAVTRGGVGPFSDPVKIFIPAHGWVDYAPSSTPAPGNADPVLII

SEQ ID NO: 3 - protein, 507 aa, Artificial Sequence
  Cyno MerTK ECD
MGLAPLPLPLLLGLFLPALWSRAITEAREEAKPYPLFPGPLPGSLQTDHTSLLSLPHTSGYQPALMFSPTQPGRPYTGNVAIPRVTSAGSKLLPPLAFKHTVGHIILSEHKDVKFNCSISVPNIYQDTTISWWKDGKELLGAHHAITQFYPDDEVTAIIASFSITSVQRSDNGSYICKMKINNEEIVSDPIYIEVQGLPHFTKQPESMNVTRNTAFNLTCQAVGPPEPVNIFWVQNSSRVNEQPEKSPSVLTVPGLTEMAVFSCEAHNDKGLTVSKGVQINIKAIPSPPTEVSIHNSTAHSILISWVPGFDGYSPFRNCSVQVKEVDPLSNGSVMIFNTSASPHMYQIKQLQALANYSIGVSCMNEIGWSAVSPWILASTTEGAPSVAPLNVTVFLNESRDNVDIRWMKPLTKRQAGELVGYRISHVWQSAGISKELLEEVGQNNSRAQISVQVHNATCTVRIAAVTKGGVGPFSDPVKIFIPAHGWVDHAPSSTPAPGNADPVLII

SEQ ID NO: 4 - protein, 497 aa, Artificial Sequence
  Murine MerTK ECD
MVLAPLLLGLLLLPALWSGGTAEKWEETELDQLFSGPLPGRLPVNHRPFSAPHSSRDQLPPPQTGRSHPAHTAAPQVTSTASKLLPPVAFNHTIGHIVLSEHKNVKFNCSINIPNTYQETAGISWWKDGKELLGAHHSITQFYPDEEGVSIIALFSIASVQRSDNGSYFCKMKVNNREIVSDPIYVEVQGLPYFIKQPESVNVTRNTAFNLTCQAVGPPEPVNIFWVQNSSRVNEKPERSPSVLTVPGLTETAVFSCEAHNDKGLTVSKGVHINIKVIPSPPTEVHILNSTAHSILVSWVPGFDGYSPLQNCSIQVKEADRLSNGSVMVFNTSASPHLYEIQQLQALANYSIAVSCRNEIGWSAVSPWILASTTEGAPSVAPLNITVFLNESNNILDIRWTKPPIKRQDGELVGYRISHVWESAGTYKELSEEVSQNGSWAQIPVQIHNATCTVRIAAITKGGIGPFSEPVNIIIPEHSKVDYAPSSTPAPGNTDSM

SEQ ID NO: 5 - protein, 118 aa, Artificial Sequence
  Heavy Chain Variable - MTK-15
QIQLVQSGPELKKPGETVKISCKASGYTFTNYGVHWVKQAPGKGLKWMGWINTYTGEPTYADDFKGRFAFSLETSASTAYLQINNLKNEDMATYFCARGNRYAYMDYWGQGTSVTVSS

SEQ ID NO: 6 - protein, 106 aa, Artificial Sequence
  Light Chain Variable - MTK-15
QIVLSQSPAILSASPGEKVTMTCRASSSVSYMHWYQQKSGSSPKPWIYATSNLASGVPARFSGSGSGTSYSLTISRVEAEDAATYYCQQWSSNPRTFGGGTKLEIK

SEQ ID NO: 7 - protein, 120 aa, Artificial Sequence
  Heavy Chain Variable - MTK-16
LIQLVQSGPELKKPGETVKISCKASGYTFTNHGMNWVKQDPGKGLKWMGWINTYTGEPTYADDFKGRFVFSMETSASAAFLQINNLKNEDTATYFCARKGVTAARYFDYWGQGTTLTVSS

SEQ ID NO: 8 - protein, 107 aa, Artificial Sequence
  Light Chain Variable - MTK-16
DIVMTQSPKFMSTSVGDRVSITCKASQNVRTAVAWYKKKPGQSPKALINLASNRHTGVPDRFTGSGSGTDFTLTISNVQSEDLADYFCLQHWNYPLTFGGGTKLEIK

SEQ ID NO: 9 - protein, 120 aa, Artificial Sequence
  Heavy Chain Variable - MTK-16.2
QVQLVQSGSELKKPGASVKVSCKASGYTFTNHGMNWVRQAPGQGLEWMGWINTYTGEPTYADDFKGRFVFSLDTSVSTAYLQISSLKAEDTAVYYCARKGVTAARYFDYWGQGTLVTVSS

SEQ ID NO: 10 - protein, 107 aa, Artificial Sequence
  Light Chain Variable - MTK-16.2
DIQMTQSPSSLSASVGDRVTITCRASQNVRTAVAWYQQKPGKAPKRLIYLASNRHTGVPSRFSGSGSGTEFTLTISNLQPEDFATYYCLQHWNYPLTFGGGTKLEIK

SEQ ID NO: 11 - protein, 119 aa, Artificial Sequence
  Heavy Chain Variable - MTK-33
QVQLQQPGPELVRPGTSVKLSCKASGYTFTSYWMHWIKQRPGQGLEWIGVIDPSDNYINYNQKFKGKATLTVDTSSSTAYLQLSSLTSEDSAVYYCAREAGTRGYFDYWGQGTTLTVSS

SEQ ID NO: 12 - protein, 107 aa, Artificial Sequence
  Light Chain Variable - MTK-33
SIVMTQTPKFLLVSAGDRVIITCKASQSVSNTVAWYQQKPGQSPKLLIYYASNRYTGVPDRFTGSGYGTDFTFTISTVQAEDLAVYFCQQDYRSPFTFGSGTQLEMK

SEQ ID NO: 13 - protein, 119 aa, Artificial Sequence
  Heavy Chain Variable - MTK-33.11
QVQLVQSGAEVKKPGASVKVSCKASGYTFTSYWMHWVRQAPGQGLEWIGVIDPSDNYINYNQKFQGRVTMTRDTSTSTVYMELSSLRSEDTAVYYCAREAWTRGYFDYWGQGTLVTVSS

SEQ ID NO: 14 - protein, 107 aa, Artificial Sequence
  Light Chain Variable - MTK-33.11
DIQMTQSPSSLSASVGDRVTITCQASRSVRNTVAWYQQKPGKAPKLLIYYASNRYTGVPSRFSGSGSGTDFTFTISSLQPEDIATYYCQQDYRSPFTFGQGTKLEIK

SEQ ID NO: 15 - protein, 448 aa, Artificial Sequence
  MTK-15 LALAPS heavy chain
QIQLVQSGPELKKPGETVKISCKASGYTFTNYGVHWVKQAPGKGLKWMGWINTYTGEPTYADDFKGRFAFSLETSASTAYLQINNLKNEDMATYFCARGNRYAYMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 213 aa, Artificial Sequence
  MTK-15 light chain
QIVLSQSPAILSASPGEKVTMTCRASSSVSYMHWYQQKSGSSPKPWIYATSNLASGVPARFSGSGSGTSYSLTISRVEAEDAATYYCQQWSSNPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 450 aa, Artificial Sequence
  MTK-16.2 wild-type IgG1 heavy chain
QVQLVQSGSELKKPGASVKVSCKASGYTFTNHGMNWVRQAPGQGLEWMGWINTYTGEPTYADDFKGRFVFSLDTSVSTAYLQISSLKAEDTAVYYCARKGVTAARYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 450 aa, Artificial Sequence
  MTK-16.2 LALAPS heavy chain
QVQLVQSGSELKKPGASVKVSCKASGYTFTNHGMNWVRQAPGQGLEWMGWINTYTGEPTYADDFKGRFVFSLDTSVSTAYLQISSLKAEDTAVYYCARKGVTAARYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 450 aa, Artificial Sequence
  MTK-16.2 NSLF heavy chain
QVQLVQSGSELKKPGASVKVSCKASGYTFTNHGMNWVRQAPGQGLEWMGWINTYTGEPTYADDFKGRFVFSLDTSVSTAYLQISSLKAEDTAVYYCARKGVTAARYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 447 aa, Artificial Sequence
  MTK-16.2 IgG4 heavy chain
QVQLVQSGSELKKPGASVKVSCKASGYTFTNHGMNWVRQAPGQGLEWMGWINTYTGEPTYADDFKGRFVFSLDTSVSTAYLQISSLKAEDTAVYYCARKGVTAARYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 214 aa, Artificial Sequence
  MTK-16.2 light chain
DIQMTQSPSSLSASVGDRVTITCRASQNVRTAVAWYQQKPGKAPKRLIYLASNRHTGVPSRFSGSGSGTEFTLTISNLQPEDFATYYCLQHWNYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 449 aa, Artificial Sequence
  MTK-33.11 wild-type IgG1 heavy chain
QVQLVQSGAEVKKPGASVKVSCKASGYTFTSYWMHWVRQAPGQGLEWIGVIDPSDNYINYNQKFQGRVTMTRDTSTSTVYMELSSLRSEDTAVYYCAREAWTRGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 449 aa, Artificial Sequence
  MTK-33.11 LALAPS heavy chain
QVQLVQSGAEVKKPGASVKVSCKASGYTFTSYWMHWVRQAPGQGLEWIGVIDPSDNYINYNQKFQGRVTMTRDTSTSTVYMELSSLRSEDTAVYYCAREAWTRGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 449 aa, Artificial Sequence
  MTK-33.11 NSLF heavy chain
QVQLVQSGAEVKKPGASVKVSCKASGYTFTSYWMHWVRQAPGQGLEWIGVIDPSDNYINYNQKFQGRVTMTRDTSTSTVYMELSSLRSEDTAVYYCAREAWTRGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 446 aa, Artificial Sequence
  MTK-33.11 IgG4 heavy chain
QVQLVQSGAEVKKPGASVKVSCKASGYTFTSYWMHWVRQAPGQGLEWIGVIDPSDNYINYNQKFQGRVTMTRDTSTSTVYMELSSLRSEDTAVYYCAREAWTRGYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 214 aa, Artificial Sequence
  MTK-33.11 light chain
DIQMTQSPSSLSASVGDRVTITCQASRSVRNTVAWYQQKPGKAPKLLIYYASNRYTGVPSRFSGSGSGTDFTFTISSLQPEDIATYYCQQDYRSPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 448 aa, Artificial Sequence
  Anti-PDL1 Wildtype IgG1 Heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 448 aa, Artificial Sequence
  Anti-PDL1 LALAPS heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 448 aa, Artificial Sequence
  Anti-PDL1 NSLF Heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 445 aa, Artificial Sequence
  Anti-PDL1 IgG4 Heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 214 aa, Artificial Sequence
  Anti-PDL1 light chain
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 454 aa, Artificial Sequence
  MTK-16.2 IgG1 knob Heavy chain
QVQLVQSGSELKKPGASVKVSCKASGYTFTNHGMNWVRQAPGQGLEWMGWINTYTGEPTYADDFKGRFVFSLDTSVSTAYLQISSLKAEDTAVYYCARKGVTAARYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASELFPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 450 aa, Artificial Sequence
  MTK-16.2 LALAPS knob Heavy chain
QVQLVQSGSELKKPGASVKVSCKASGYTFTNHGMNWVRQAPGQGLEWMGWINTYTGEPTYADDFKGRFVFSLDTSVSTAYLQISSLKAEDTAVYYCARKGVTAARYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 450 aa, Artificial Sequence; identical to SEQ ID NO: 19
  MTK-16.2 NSLF knob Heavy chain

SEQ ID NO: 35 - protein, 447 aa, Artificial Sequence
  MTK-16.2 IgG4 knob
QVQLVQSGSELKKPGASVKVSCKASGYTFTNHGMNWVRQAPGQGLEWMGWINTYTGEPTYADDFKGRFVFSLDTSVSTAYLQISSLKAEDTAVYYCARKGVTAARYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 447 aa, Artificial Sequence
  MTK-16.2 IgG4 knob S228P Heavy chain
QVQLVQSGSELKKPGASVKVSCKASGYTFTNHGMNWVRQAPGQGLEWMGWINTYTGEPTYADDFKGRFVFSLDTSVSTAYLQISSLKAEDTAVYYCARKGVTAARYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 449 aa, Artificial Sequence
  MTK-33.11 IgG1 knob Heavy chain
QVQLVQSGAEVKKPGASVKVSCKASSYSFTSYWMHWVRQAPGQGLEWIGVIDPSDNYINYNQKFRGRVTMTRDTSTSTVYMELSSLRSEDTAVYYCAREAGTRGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 449 aa, Artificial Sequence
  MTK-33.11 LALAPS knob Heavy chain
QVQLVQSGAEVKKPGASVKVSCKASSYSFTSYWMHWVRQAPGQGLEWIGVIDPSDNYINYNQKFRGRVTMTRDTSTSTVYMELSSLRSEDTAVYYCAREAGTRGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 449 aa, Artificial Sequence
  MTK-33.11 NSLF knob Heavy chain
QVQLVQSGAEVKKPGASVKVSCKASSYSFTSYWMHWVRQAPGQGLEWIGVIDPSDNYINYNQKFRGRVTMTRDTSTSTVYMELSSLRSEDTAVYYCAREAGTRGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 446 aa, Artificial Sequence
  MTK-33.11 IgG4 knob Heavy chain
QVQLVQSGAEVKKPGASVKVSCKASSYSFTSYWMHWVRQAPGQGLEWIGVIDPSDNYINYNQKFRGRVTMTRDTSTSTVYMELSSLRSEDTAVYYCAREAGTRGYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 446 aa, Artificial Sequence
  MTK-33.11 IgG4 knob S228P Heavy chain
QVQLVQSGAEVKKPGASVKVSCKASSYSFTSYWMHWVRQAPGQGLEWIGVIDPSDNYINYNQKFRGRVTMTRDTSTSTVYMELSSLRSEDTAVYYCAREAGTRGYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 448 aa, Artificial Sequence
  Anti-PDL1 IgG hole heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 43 - protein, 448 aa, Artificial Sequence
  Anti-PDL1 LALAPS hole Heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 44 - protein, 448 aa, Artificial Sequence
  Anti-PDL1 NSLF hole Heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 45 - protein, 445 aa, Artificial Sequence
  Anti-PDL1 IgG4 hole Heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 46 - protein, 445 aa, Artificial Sequence
  Anti-PDL1 IgG4 hole S228P Heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 47 - protein, 227 aa, Artificial Sequence
  IgG1 wildtype Fc hole
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 48 - protein, 227 aa, Artificial Sequence
  IgG1 LALAPS Fc hole
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 49 - protein, 227 aa, Artificial Sequence
  IgG1 NSLF Fc hole
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 50 - protein, 229 aa, Artificial Sequence
  IgG4 wildtype Fc hole
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 51 - protein, 229 aa, Artificial Sequence
  IgG4 S228P Fc hole
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 52 - protein, 118 aa, Artificial Sequence [sequence omitted]

SEQ ID NO: 53 - protein, 107 aa, Artificial Sequence [sequence omitted]

SEQ ID NO: 54 - protein, 12 aa, Artificial Sequence
  Wildtype hinge region of huIgG4
ESKYGPPCPSCP

SEQ ID NO: 55 - protein, 12 aa, Artificial Sequence
  S228P amino acid substitution of IgG4 hinge region
ESKYGPPCPPCP

(19) United States
(12) Patent Application Publication
(10) Pub. No.: US 2024/0279341 A1
(43) Pub. Date: Aug. 22, 2024

(54) BISPECIFIC ANTI-MERTK AND ANTI-PDL1 ANTIBODIES AND METHODS OF USE THEREOF

(71) Applicant: ALECTOR LLC, South San Francisco, CA (US)

(72) Inventors: Seung-Joo LEE, South San Francisco, CA (US); Tarangsri NIVITCHANYONG, Castro Valley, CA (US); Wei-Hsien HO, Belmont, CA (US); Wei LI, Pacifica, CA (US); Marina K. ROELL, Concord, CA (US); Spencer LIANG, South San Francisco, CA (US)

(21) Appl. No.: 18/570,865

(22) PCT Filed: Jun. 15, 2022
(86) PCT No.: PCT/US2022/033632

§ 371 Date: Dec. 15, 2023

Related U.S. Application Data
(60) Provisional application No. 63/211,437, filed on Jun. 16, 2021.

(51) Int. Cl. C07K 16/28; A61K 39/00; A61P 35/00; C07K 16/40

(52) U.S. Cl. CPC C07K 16/2827; A61P 35/00; C07K 16/40; A61K 2039/505; C07K 2317/24; C07K 2317/31; C07K 2317/33; C07K 2317/54; C07K 2317/76; C07K 2317/92

(57) ABSTRACT

The present disclosure is generally directed to compositions that include bispecific antibodies, e.g., monoclonal antibodies, that specifically bind a MerTK polypeptide (e.g., a mammalian MerTK or human MerTK) and a PDL1 polypeptide (e.g. a mammalian PDL1 polypeptide or human PDL1 polypeptide), and use of such compositions in treating an individual in need thereof.

### CROSS-REFERENCE TO RELATED APPLICATIONS

This application claims the priority benefit of U.S. Provisional Appl. No. 63/211,437, filed Jun. 16, 2021, which is hereby incorporated by reference herein in its entirety.

### SUBMISSION OF SEQUENCE LISTING ON ASCII TEXT FILE

The content of the following submission of ASCII text file is incorporated herein by reference in its entirety: a computer readable form (CRF) of the Sequence Listing (file name: 4503_020 PC01_Seqlisting_ST25.txt; size: 163,928 bytes; and date of creation: Jun. 14, 2022)

### FIELD OF THE PRESENT DISCLOSURE

The present disclosure relates to bispecfic anti-MerTK and anti-PDL1 antibodies and uses (e.g., therapeutic uses) of such antibodies.

### BACKGROUND OF THE PRESENT DISCLOSURE

Mer Tyrosine Kinase (MerTK) belongs to the TAM (Tyro3, Axl, and MerTK) family of receptor tyrosine kinases. MerTK is a single-pass type 1 transmembrane protein with an extracellular domain having two immunoglobulin (Ig)-like and two fibronectin (FN) type III motifs (Graham et al, 2014, Nat Rev Cancer, 14:769-785; Rothlin et al, 2015, Annu Rev Immunol, 33:355-391).

Several ligands of MerTK have been identified, including Protein S (ProS or ProS1), Growth arrest specific gene 6 (Gas6), Tubby, Tubby-like protein 1 (TULP-1), and Galectin-3. MerTK transduces signals from the extracellular space via activation following ligand binding, leading to MerTK tyrosine auto-phosphorylation (Cummings et al, 2013, Clin Cancer Res, 19:5275-5280; Verma et al, 2011, Mol Cancer Ther, 10:1763-1773) and subsequent ERK and AKT-associated signal transduction.

MerTK regulates various physiological processes including cell survival, migration, and differentiation. MerTK ligands ProS and Gas6 contribute to several oncogenic processes, such as cell survival, invasion, migration, chemo-resistance, and metastasis, in which their expression often correlates with poor clinical outcomes. Additionally, MerTK is implicated in numerous cancers, and MerTK or ProS deficiency is associated with anti-tumor effects (Cook et al, 2013, J Clin Invest, 123:3231-3242; Ubil et al, 2018, J Clin Invest, 128:2356-2369; Huey et al, 2016, Cancers, 8:101). However, MerTK is also expressed on retinal pigment epithelial cells and plays a critical role in clearing shed photoreceptor outer segment in the eye; loss of function mutations in MerTK result in retinitis pigmentosa and other retinal dystrophies (see, e.g. Al-khersan et al, Graefes Arch Clin Exp Ophthalmol, 2017, 255:1613-1619; Lorach et al, Nature Scientific Reports, 2018, 8:11312; Audo et al, Human Mutation, Wiley, 2018, 39:997-913; LaVail et al, Adv Exp Med Biol, 2016, 854:487-493).

MerTK plays an essential role in phagocytosis of apoptotic cells (efferocytosis) by phagocytic cells, leading to M2-like macrophage polarization, production of anti-inflammatory cytokines, and promoting an immunosuppressive tumor microenvironment. Reducing efferocytosis by phagocytic cells increases M1-like macrophage polarization, leading to the production of pro-inflammatory cytokines and an immune-active milieu. Modulating efferocytosis can provide an effective means for anti-tumor activity.

Anti-MerTK antibodies have been previously described in, e.g., International Patent Application Publication Nos: WO2020/214995, WO2020/076799, WO2020/106461, WO2020/176497, WO2019/084307, WO2019/005756, WO2016/106221, WO2016/001830, WO2009/062112, and WO2006/058202; International Patent Application Serial No. PCT/US2020/064640 (WO 2021/119508); and in, e.g., Dayoub and Brekken, 2020, Cell Communications and Signaling, 18:29; Zhou et al, 2020, Immunity, 52:1-17; Kedage et al, 2020, MABS, 12:e1685832; Cummings et al, 2014, Oncotarget, 5:10434-10445.

There is a need for novel therapeutic anti-MerTK antibodies that are effective at treating conditions such as cancer.

All references cited herein, including patent applications and publications, are hereby incorporated by reference in their entirety.

### SUMMARY OF THE PRESENT DISCLOSURE

International Patent Application Serial No. PCT/US2020/064640 (WO 2021/119508) disclosed that administration of an anti-MerTK antibody in combination with an anti-PDL1 antibody in a mouse tumor model showed greater reduction of tumor volume compared to administration of antibody alone, indicating better efficacy was obtained by combination therapy. However, administration of two separate antibodies places a burden on both clinicians and patients. The present disclosure meets this need by providing bispecific anti-MerTK:anti-PDL1 antibodies having improved efficacy at mediating anti-tumor immunity.

It is to be understood that one, some, or all of the properties of the various embodiments described herein may be combined to form other embodiments of the present disclosure. These and other aspects of the disclosure will become apparent to one of skill in the art. These and other aspects of the disclosure are further described by the detailed description that follows.

In some aspects, provided herein is a bispecific antibody that binds to human Mer Tyrosine Kinase (MerTK) and programmed death-ligand 1 (PDL1), wherein the bispecific antibody contains a first antigen-binding domain that binds to human MerTk and a second antigen-binding domain that binds to PDL1.

In some aspects, the first antigen-binding domain binds to the Ig1 domain of MerTK protein.

In some aspects, the first antigen binding domain competitively inhibits binding to the MerTK of an antibody comprising a variable heavy chain comprising the amino acid sequence of SEQ ID NO:9 and a variable light chain comprising the amino acid sequence of SEQ ID NO:10.

In some aspects, the first antigen binding domain binds to the same MerTK epitope as an antibody comprising a variable heavy chain comprising the amino acid sequence of SEQ ID NO:9 and a variable light chain comprising the amino acid sequence of SEQ ID NO:10.

In some aspects, the first antigen binding domain an HVR-H1 comprising amino acids 31-35 of SEQ ID NO:9, an HVR-H2 comprising amino acids 50-66 of SEQ ID NO:9, an HVR-H3 comprising amino acids 99-109 of SEQ ID NO:9, an HVR-L1 comprising amino acids 24-34 of SEQ ID NO:10, an HVR-L2 comprising amino acids 50-56 of SEQ ID NO: 10, and an HVR-L3 comprising amino acids 89-97 of SEQ ID NO:10.

In some aspects, the first antigen binding domain comprises the HVRs of the 16.2 antibody, optionally wherein the HVRs are the Kabat-defined HVRs, the Chothia-defined HVRs, the AbM-defined HVRs, or the contact-defined HVRs

In some aspects, the first antigen binding domain comprises a variable heavy chain comprising an amino acid sequence that is at least 85%, at least 90%, or at least 95% identical to the amino acid sequence of SEQ ID NO:9.

In some aspects, the first antigen binding domain comprises a variable light chain comprising an amino acid sequence that is at least 85%, at least 90%, or at least 95% identical to the amino acid sequence of SEQ ID NO:10.

In some aspects, the first antigen binding domain comprises a variable heavy chain comprising the amino acid sequence of SEQ ID NO:9 and/or a variable light chain comprising the amino acid sequence of SEQ ID NO: 10.

In some aspects, the first antigen binding domain binds to the same MerTK epitope as an antibody comprising a variable heavy chain comprising the amino acid sequence of SEQ ID NO:13 and a variable light chain comprising the amino acid sequence of SEQ ID NO: 14.

In some aspects, the first antigen binding domain competitively inhibits binding to the MerTK of an antibody comprising a variable heavy chain comprising the amino acid sequence of SEQ ID NO: 13 and a variable light chain comprising the amino acid sequence of SEQ ID NO: 14.

In some aspects, the first antigen binding domain comprises an HVR-H1 comprising amino acids 31-35 of SEQ ID NO: 13, an HVR-H2 comprising amino acids 50-66 of SEQ ID NO: 13, an HVR-H3 comprising amino acids 99-108 of SEQ ID NO: 13, an HVR-L1 comprising amino acids 24-34 of SEQ ID NO: 14, an HVR-L2 comprising amino acids 50-56 of SEQ ID NO: 14, and an HVR-L3 comprising amino acids 89-97 of SEQ ID NO: 14.

In some aspects, the first antigen binding domain comprises the HVRs of the 13.11 antibody, optionally wherein the HVRs are the Kabat-defined HVRs, the Chothia-defined HVRs, the AbM-defined HVRs, or the contact-defined HVRs.

In some aspects, the first antigen binding domain comprises a variable heavy chain comprising an amino acid sequence that is at least 85%, at least 90%, or at least 95% identical to the amino acid sequence of SEQ ID NO: 13.

In some aspects, the first antigen binding domain comprises a variable light chain comprising an amino acid sequence that is at least 85%, at least 90%, or at least 95% identical to the amino acid sequence of SEQ ID NO: 14.

In some aspects, the first antigen binding domain comprises a variable heavy chain comprising the amino acid sequence of SEQ ID NO:13 and a variable light chain comprising the amino acid sequence of SEQ ID NO: 14.

In some aspects, the second antigen binding domain binds to the same PDL1 epitope as an antibody comprising a variable heavy chain comprising the amino acid sequence of SEQ ID NO:52 and a variable light chain comprising the amino acid sequence of SEQ ID NO:53.

In some aspects, the second antigen binding domain competitively inhibits binding to the PDL1 of an antibody comprising a variable heavy chain comprising the amino acid sequence of SEQ ID NO:52 and a variable light chain comprising the amino acid sequence of SEQ ID NO:53.

In some aspects, the second antigen binding domain comprises an HVR-H1 comprising amino acids 31-35 of SEQ ID NO:52, an HVR-H2 comprising amino acids 50-66 of SEQ ID NO:52, an HVR-H3 comprising amino acids 99-107 of SEQ ID NO:52, an HVR-L1 comprising amino acids 24-34 of SEQ ID NO:53, an HVR-L2 comprising amino acids 50-56 of SEQ ID NO:53, and an HVR-L3 comprising amino acids 89-97 of SEQ ID NO:53

In some aspects, the second antigen binding domain comprises the HVRs of the atezolizumab antibody, optionally wherein the HVRs are the Kabat-defined HVRs, the Chothia-defined HVRs, the AbM-defined HVRs, or the contact-defined HVRs.

In some aspects, the second antigen binding domain comprises a variable heavy chain comprising an amino acid sequence that is at least 85%, at least 90%, or at least 95% identical to the amino acid sequence of SEQ ID NO:52.

In some aspects, the second antigen binding domain comprises a variable light chain comprising an amino acid sequence that is at least 85%, at least 90%, or at least 95% identical to the amino acid sequence of SEQ ID NO:53.

In some aspects, the second antigen binding domain comprises a variable heavy chain comprising the amino acid sequence of SEQ ID NO:52 and a variable light chain comprising the amino acid sequence of SEQ ID NO:53.

In some aspects, the bispecific antibody is of the IgG class, the IgM class, or the IgA class.

In some aspects, the bispecific antibody is of the IgG class, optionally wherein the bispecific antibody has an IgG1, an IgG2, or an IgG4 isotype.

In some aspects, the bispecific antibody is an IgG1 antibody.

In some aspects, the bispecific antibody is an IgG4 antibody.

In some aspects, the bispecific antibody (i) has two arms comprising different antigen-binding domains, (ii) is a single chain antibody that has specificity to two different epitopes, (iii) is a chemically-linked bispecific (Fab′)2 fragment, (iv) is a fusion of two single chain diabodies resulting in a tetravalent bispecific antibody that has two binding sites for each of the target antigens, (v) is a combination of scFvs with a diabody resulting in a multivalent molecule, (vi) comprises two scFvs fused to both termini of a human Fab-arm, or (vii) is a diabody.

In some aspects, the bispecific antibody is a kappa-lambda body, a dual-affinity re-targeting molecule (DART), a knob-in-hole antibody, a strand-exchange engineered domain body (SEEDbody), or a DuoBody.

In some aspects, the bispecific antibody comprises an Fc region comprising a first polypeptide and a second polypeptide. In some aspects, the first polypeptide comprises the amino acid substitution T366Y, and the second polypeptide comprises the amino acid substation Y407T. In some aspects, the first polypeptide comprises the amino acid substitution T366W, and the second polypeptide comprises the amino acid substitutions T366S, L368W, and Y407V. In some aspects, the first polypeptide comprises the amino acid substitution T366W, and the second polypeptide comprises the amino acid substitutions T366S, L368A, and Y407V. In some aspects, the first polypeptide comprises the amino acid substitutions T366W, and S354C and the second polypeptide comprises the amino acid substitutions T366S, L368A, Y407V, and Y349C. In some aspects, the first polypeptide comprises the amino acid substitutions T350V, L351Y, F405A, Y407V, and the second polypeptide comprises the amino acid substitutions T350V, T366L, K392L, and T394W. In some aspects, the first polypeptide comprises the amino acid substitutions K360D, D399M, and Y407A, and the second polypeptide comprises the amino acid substitutions E345R, Q347R, T366V, and K409V. In some aspects, the first polypeptide comprises the amino acid substitutions K409D and K392D, and the second polypeptide comprises the amino acid substitutions D399K and E356K. In some aspects, the first polypeptide comprises the amino acid substitutions K360E and K409W, and the second polypeptide comprises the amino acid substitutions Q347R, D399V, and F405T. In some aspects, the first polypeptide comprises the amino acid substitutions L360E, K409W, and Y349C, and the second polypeptide comprises the amino acid substitutions Q347R, D399V, F405T, and S354C. In some aspects, the first polypeptide comprises the amino acid substitutions K370E and K409W, and the second polypeptide comprises the amino acid substitutions E357N, D399V, and F405T. In some aspects, the substitution is according to EU numbering.

In some aspects, the bispecific antibody comprises a knob mutation and a hole mutation.

In some aspects, the knob mutation comprises the amino acid substitution T366W according to EU numbering.

In some aspects, the hole mutation comprises the amino acids substitutions T366S, L368A, and Y407V according to EU numbering.

In some aspects, the bispecific antibody comprises an Fc region comprising an amino acid substitution, addition, or deletion that promotes heterodimerization.

In some aspects, the bispecific antibody comprises the amino acid substitution S228P according to EU numbering.

In some aspects, the bispecific antibody comprises the amino acid substitutions L234A, L235A, and P331S (LALAPS) accordingly to EU numbering.

In some aspects, the bispecific antibody comprises the amino acid substitutions N325S and L328F (NSLF) according to EU numbering.

In some aspects, the bispecific antibody comprises a heavy chain comprising amino acids 1-449 of SEQ ID NO: 17 or the amino acid sequence of SEQ ID NO:17.

In some aspects, the bispecific antibody comprises a heavy chain comprising amino acids 1-449 of SEQ ID NO:18 or the amino acid sequence of SEQ ID NO:18.

In some aspects, the bispecific antibody comprises a heavy chain comprising amino acids 1-449 of SEQ ID NO: 19 or the amino acid sequence of SEQ ID NO:19.

In some aspects, the bispecific antibody comprises a heavy chain comprising amino acids 1-446 of SEQ ID NO:20 or the amino acid sequence of SEQ ID NO:20.

In some aspects, the bispecific antibody comprises a heavy chain comprising amino acids 1-453 of SEQ ID NO:32 or the amino acid sequence of SEQ ID NO:32.

In some aspects, the bispecific antibody comprises a heavy chain comprising amino acids 1-449 of SEQ ID NO:33 or the amino acid sequence of SEQ ID NO:33.

In some aspects, the bispecific antibody comprises a heavy chain comprising amino acids 1-449 of SEQ ID NO:34 or the amino acid sequence of SEQ ID NO:34.

In some aspects, the bispecific antibody comprises a heavy chain comprising amino acids 1-446 of SEQ ID NO:35 or the amino acid sequence of SEQ ID NO:35.

In some aspects, the bispecific antibody comprises a heavy chain comprising amino acids 1-446 of SEQ ID NO:36 or the amino acid sequence of SEQ ID NO:36.

In some aspects, the bispecific antibody comprises a light chain comprising the amino acid sequence of SEQ ID NO:21.

In some aspects, the bispecific antibody comprises a heavy chain comprising amino acids 1-448 of SEQ ID NO:22 or the amino acid sequence of SEQ ID NO:22.

In some aspects, the bispecific antibody comprises a heavy chain comprising amino acids 1-448 of SEQ ID NO:23 or the amino acid sequence of SEQ ID NO:23.

In some aspects, the bispecific antibody comprises a heavy chain comprising amino acids 1-448 of SEQ ID NO:24 or the amino acid sequence of SEQ ID NO:24.

In some aspects, the bispecific antibody comprises a heavy chain comprising amino acids 1-445 of SEQ ID NO:25 or the amino acid sequence of SEQ ID NO:25.

In some aspects, the bispecific antibody comprises a heavy chain comprising amino acids 1-448 of SEQ ID NO:37 or the amino acid sequence of SEQ ID NO:37.

In some aspects, the bispecific antibody comprises a heavy chain comprising amino acids 1-448 of SEQ ID NO:38 or the amino acid sequence of SEQ ID NO:38.

In some aspects, the bispecific antibody comprises a heavy chain comprising amino acids 1-448 of SEQ ID NO:39 or the amino acid sequence of SEQ ID NO:39.

In some aspects, the bispecific antibody comprises a heavy chain comprising amino acids 1-445 of SEQ ID NO:40 or the amino acid sequence of SEQ ID NO:40.

In some aspects, the bispecific antibody comprises a heavy chain comprising amino acids 1-445 of SEQ ID NO:41 or the amino acid sequence of SEQ ID NO:41.

In some aspects, the bispecific antibody comprises a light chain comprising the amino acid sequence of SEQ ID NO:26.

In some aspects, the bispecific antibody comprises a heavy chain comprising amino acids 1-447 of SEQ ID NO:27 or the amino acid sequence of SEQ ID NO:27.

In some aspects, the bispecific antibody comprises a heavy chain comprising amino acids 1-447 of SEQ ID NO:28 or the amino acid sequence of SEQ ID NO:28.

In some aspects, the bispecific antibody comprises a heavy chain comprising amino acids 1-447 of SEQ ID NO:29 or the amino acid sequence of SEQ ID NO:29.

In some aspects, the bispecific antibody comprises a heavy chain comprising amino acids 1-444 of SEQ ID NO:30 or the amino acid sequence of SEQ ID NO:30.

In some aspects, the bispecific antibody comprises a heavy chain comprising amino acids 1-447 of SEQ ID NO:42 or the amino acid sequence of SEQ ID NO:42.

In some aspects, the bispecific antibody comprises a heavy chain comprising amino acids 1-447 of SEQ ID NO:43 or the amino acid sequence of SEQ ID NO:43.

In some aspects, the bispecific antibody comprises a heavy chain comprising amino acids 1-447 of SEQ ID NO:44 or the amino acid sequence of SEQ ID NO:44.

In some aspects, the bispecific antibody comprises a heavy chain comprising amino acids 1-444 of SEQ ID NO:45 or the amino acid sequence of SEQ ID NO:45.

In some aspects, the bispecific antibody comprises a heavy chain comprising amino acids 1-444 of SEQ ID NO:46 or the amino acid sequence of SEQ ID NO:46.

In some aspects, the bispecific antibody comprises a light chain comprising the amino acid sequence of SEQ ID NO:31.

In some aspects, the bispecific antibody comprises a heavy chain constant region comprising the amino acid sequence of SEQ ID NO:47.

In some aspects, the bispecific antibody comprises a heavy chain constant region comprising the amino acid sequence of SEQ ID NO:48.

In some aspects, the bispecific antibody comprises a heavy chain constant region comprising the amino acid sequence of SEQ ID NO:49.

In some aspects, the bispecific antibody comprises a heavy chain constant region comprising the amino acid sequence of SEQ ID NO:50.

In some aspects, the bispecific antibody comprises a heavy chain constant region comprising the amino acid sequence of SEQ ID NO:51.

In some aspects, the bispecific antibody comprises a heavy chain comprising amino acids 1-449 of SEQ ID NO: 17 or the amino acid sequence of SEQ ID NO:17, a light chain comprising the amino acid sequence of SEQ ID NO:21, a heavy chain comprising amino acids 1-447 of SEQ ID NO:27 or the amino acid sequence of SEQ ID NO:27 and a light chain comprising the amino acid sequence of SEQ ID NO:31.

In some aspects, the bispecific antibody comprises a heavy chain comprising amino acids 1-449 of SEQ ID NO: 18 or the amino acid sequence of SEQ ID NO:18, a light chain comprising the amino acid sequence of SEQ ID NO:21, a heavy chain comprising amino acids 1-447 of SEQ ID NO:28 or the amino acid sequence of SEQ ID NO:28 and a light chain comprising the amino acid sequence of SEQ ID NO:31.

In some aspects, the bispecific antibody comprises a heavy chain comprising amino acids 1-449 of SEQ ID NO: 19 or the amino acid sequence of SEQ ID NO:19, a light chain comprising the amino acid sequence of SEQ ID NO:21, a heavy chain comprising amino acids 1-447 of SEQ ID NO:29 or the amino acid sequence of SEQ ID NO:29 and a light chain comprising the amino acid sequence of SEQ ID NO:31.

In some aspects, the bispecific antibody comprises a heavy chain comprising amino acids 1-446 of SEQ ID NO:20 or the amino acid sequence of SEQ ID NO:20, a light chain comprising the amino acid sequence of SEQ ID NO:21, a heavy chain comprising amino acids 1-444 of SEQ ID NO:30 or the amino acid sequence of SEQ ID NO:30 and a light chain comprising the amino acid sequence of SEQ ID NO:31.

In some aspects, the bispecific antibody comprises a heavy chain comprising amino acids 1-448 of SEQ ID NO:22 or the amino acid sequence of SEQ ID NO:22, a light chain comprising the amino acid sequence of SEQ ID NO:26, a heavy chain comprising amino acids 1-447 of SEQ ID NO:27 or the amino acid sequence of SEQ ID NO:27 and a light chain comprising the amino acid sequence of SEQ ID NO:31.

In some aspects, the bispecific antibody comprises a heavy chain comprising amino acids 1-448 of SEQ ID NO:23 or the amino acid sequence of SEQ ID NO:23, a light chain comprising the amino acid sequence of SEQ ID NO:26, a heavy chain comprising amino acids 1-447 of SEQ ID NO:28 or the amino acid sequence of SEQ ID NO:28 and a light chain comprising the amino acid sequence of SEQ ID NO:31.

In some aspects, the bispecific antibody comprises a heavy chain comprising amino acids 1-448 of SEQ ID NO:24 or the amino acid sequence of SEQ ID NO:24, a light chain comprising the amino acid sequence of SEQ ID NO:26, a heavy chain comprising amino acids 1-447 of SEQ ID NO:29 or the amino acid sequence of SEQ ID NO:29 and a light chain comprising the amino acid sequence of SEQ ID NO:31.

In some aspects, the bispecific antibody comprises a heavy chain comprising amino acids 1-445 of SEQ ID NO:25 or the amino acid sequence of SEQ ID NO:25, a light chain comprising the amino acid sequence of SEQ ID NO:26, a heavy chain comprising amino acids 1-444 of SEQ ID NO:30 or the amino acid sequence of SEQ ID NO:30 and a light chain comprising the amino acid sequence of SEQ ID NO:31.

In some aspects, the bispecific antibody comprises a heavy chain comprising amino acids 1-453 of SEQ ID NO:32 or the amino acid sequence of SEQ ID NO:32, a light chain comprising the amino acid sequence of SEQ ID NO:21, a heavy chain comprising amino acids 1-447 of SEQ ID NO:42 or the amino acid sequence of SEQ ID NO:42 and a light chain comprising the amino acid sequence of SEQ ID NO:31.

In some aspects, the bispecific antibody comprises a heavy chain comprising amino acids 1-449 of SEQ ID NO:33 or the amino acid sequence of SEQ ID NO:33, a light chain comprising the amino acid sequence of SEQ ID NO:21, a heavy chain comprising amino acids 1-447 of SEQ ID NO:43 or the amino acid sequence of SEQ ID NO:43 and a light chain comprising the amino acid sequence of SEQ ID NO:31.

In some aspects, the bispecific antibody comprises a heavy chain comprising amino acids 1-449 of SEQ ID NO:34 or the amino acid sequence of SEQ ID NO:34, a light chain comprising the amino acid sequence of SEQ ID NO:21, a heavy chain comprising amino acids 1-447 of SEQ ID NO:44 or the amino acid sequence of SEQ ID NO:44 and a light chain comprising the amino acid sequence of SEQ ID NO:31.

In some aspects, the bispecific antibody comprises a heavy chain comprising amino acids 1-446 of SEQ ID NO:35 or the amino acid sequence of SEQ ID NO:35, a light chain comprising the amino acid sequence of SEQ ID NO:21, a heavy chain comprising amino acids 1-444 of SEQ ID NO:45 or the amino acid sequence of SEQ ID NO:45 and a light chain comprising the amino acid sequence of SEQ ID NO:31.

In some aspects, the bispecific antibody comprises a heavy chain comprising amino acids 1-446 of SEQ ID NO:36 or the amino acid sequence of SEQ ID NO:36, a light chain comprising the amino acid sequence of SEQ ID NO:21, a heavy chain comprising amino acids 1-444 of SEQ ID NO:46 or the amino acid sequence of SEQ ID NO:46 and a light chain comprising the amino acid sequence of SEQ ID NO:31.

In some aspects, the bispecific antibody comprises a heavy chain comprising amino acids 1-448 of SEQ ID NO:37 or the amino acid sequence of SEQ ID NO:37, a light chain comprising the amino acid sequence of SEQ ID NO:26, a heavy chain comprising amino acids 1-447 of SEQ ID NO:42 or the amino acid sequence of SEQ ID NO:42 and a light chain comprising the amino acid sequence of SEQ ID NO:31.

In some aspects, the bispecific antibody comprises a heavy chain comprising amino acids 1-448 of SEQ ID NO:38 or the amino acid sequence of SEQ ID NO:38, a light chain comprising the amino acid sequence of SEQ ID NO:26, a heavy chain comprising amino acids 1-447 of SEQ ID NO:43 or the amino acid sequence of SEQ ID NO:43 and a light chain comprising the amino acid sequence of SEQ ID NO:31.

In some aspects, the bispecific antibody comprises a heavy chain comprising amino acids 1-448 of SEQ ID NO:39 or the amino acid sequence of SEQ ID NO:39, a light chain comprising the amino acid sequence of SEQ ID NO:26, a heavy chain comprising amino acids 1-447 of SEQ ID NO:44 or the amino acid sequence of SEQ ID NO:44 and a light chain comprising the amino acid sequence of SEQ ID NO:31.

In some aspects, the bispecific antibody comprises a heavy chain comprising amino acids 1-445 of SEQ ID NO:40 or the amino acid sequence of SEQ ID NO:40, a light chain comprising the amino acid sequence of SEQ ID NO:26, a heavy chain comprising amino acids 1-444 of SEQ ID NO:45 or the amino acid sequence of SEQ ID NO:45 and a light chain comprising the amino acid sequence of SEQ ID NO:31.

In some aspects, the bispecific antibody comprises a heavy chain comprising amino acids 1-445 of SEQ ID NO:41 or the amino acid sequence of SEQ ID NO:41, a light chain comprising the amino acid sequence of SEQ ID NO:26, a heavy chain comprising amino acids 1-444 of SEQ ID NO:46 or the amino acid sequence of SEQ ID NO:46 and a light chain comprising the amino acid sequence of SEQ ID NO:31.

In some aspects, the bispecific antibody is capable of binding MerTK and PDL1 simultaneously.

In some aspects, the bispecific antibody reduces efferocytosis by a phagocytic cell.

In some aspects, the bispecific antibody reduces efferocytosis with an IC50 value of about 4 nM to about 16 nM.

In some aspects, the bispecific antibody reduces efferocytosis with an IC50 value of about 4 nM to about 5 nM, or about 15 nM to about 16 nM.

In some aspects, the phagocytic cell is a macrophage, a tumor-associated macrophage, or a dendritic cell.

In some aspects, the phagocytic cell is a macrophage.

In some aspects, the bispecific antibody inhibits tumor growth.

In some aspects, the bispecific antibody reduces binding of ProS to MerTK.

In some aspects, the bispecific antibody reduces binding of Gas6 to MerTK.

In some aspects, the bispecific antibody reduces binding of ProS to MerTK and reduces the binding of Gas6 to MerTK.

In some aspects, the bispecific antibody binds to human MerTK with a binding affinity of about 2 nM to about 30 nM, optionally wherein the bispecific antibody binds to human MerTK with a binding affinity of about 2 nM or about 30 nM.

In some aspects, the bispecific antibody also binds to cynomolgus monkey MerTk.

In some aspects, the bispecific antibody binds to cynomolgus MerTK with a binding affinity of about 2 nM to about 30 nM, optionally wherein the bispecific antibody binds to cynomolgus MerTK with a binding affinity of about 2 nM or about 30 nM.

In some aspects, the bispecific antibody also binds to murine MerTK.

In some aspects, the bispecific antibody binds to murine MerTK with a binding affinity of about 40 nM.

In some aspects, the bispecific antibody does not bind to murine MerTK.

In some aspects, the bispecific antibody reduces Gas6-mediated phosphorylation of AKT.

In some aspects, the bispecific antibody reduces Gas6-mediated phosphorylation of AKT with an IC50 value of about 9 nM to about 13 nM, optionally wherein the bispecific antibody reduces Gas6-mediated phosphorylation of AKT with an IC50 value of about 9 nM or about 13 nM.

In some aspects, the bispecific antibody is a murine antibody, a human antibody, a humanized antibody, a monoclonal antibody, a multivalent antibody, a conjugated antibody, or a chimeric antibody.

In some aspects, provided herein is a humanized form of any of the bispecific antibodies described herein.

In some aspects, the bispecific antibody is a recombinant antibody.

In some aspects, the bispecific antibody is an isolated antibody.

In some aspects, provided herein is an isolated nucleic acid comprising a nucleic acid sequence encoding any of the bispecific antibodies described herein. In some aspects, provided herein is a vector comprising the nucleic acid.

In some aspects, provided herein is an isolated host cell comprising the nucleic acid described herein or the vector described herein.

In some aspects, the isolated host cell comprises (i) a nucleic acid comprising a nucleic acid sequence encoding the variable heavy chain of the first antigen binding domain of any of the bispecific antibodies described herein; (ii) a nucleic acid comprising a nucleic acid sequence encoding the variable light chain of the first antigen binding domain of the bispecific antibody; (iii) a nucleic acid comprising a nucleic acid sequence encoding the variable heavy chain of the second antigen binding domain of the bispecific antibody; and (iv) a nucleic acid comprising a nucleic acid sequence encoding the variable light chain of the second antigen binding domain of the bispecific antibody;

In some aspects, the isolated host cell comprises (i) a nucleic acid comprising a nucleic acid sequence encoding the heavy chain of the first antigen binding domain of the bispecific antibody; (ii) a nucleic acid comprising a nucleic acid sequence encoding the light chain of the first antigen binding domain of the bispecific antibody; (iii) a nucleic acid comprising a nucleic acid sequence encoding the heavy chain of the second antigen binding domain of any of the bispecific antibodies described herein; and (iv) a nucleic acid comprising a nucleic acid sequence encoding the light chain of the second antigen binding domain of the bispecific antibody.

In some aspects, provided herein is a method of producing a bispecific antibody that binds to human MerTK and PDL1, the method comprising culturing any of the cells described herein so that the bispecific antibody is produced.

In some aspects, the method further comprises recovering the bispecific antibody produced by the cell.

In some aspects, provided herein is a bispecific antibody produced by the methods described herein.

In some aspects, provided herein is a pharmaceutical composition comprising any of the bispecific antibodies described herein and a pharmaceutically acceptable carrier.

In some aspects, provided herein is a method of treating cancer in an individual, the method comprising administering to an individual a therapeutically effective amount of any of the bispecific antibodies described herein or the pharmaceutical composition described herein. In some aspects, the cancer is colon cancer, ovarian cancer, liver cancer, or endometrial cancer.

In some aspects, the administration does not lead to a retinal pathology in the individual.

In some aspects, provided herein is a method for detecting MerTK in a sample comprising contacting said sample with any of the bispecific antibodies described herein.

### BRIEF DESCRIPTION OF THE DRAWINGS

FIG. 1A, FIG. 1B, and FIG. 1C set forth data showing binding of certain anti-MerTK:anti-PDL1 bispecific antibodies of the present disclosure to CHO cells overexpressing human PDL1, CHO cells overexpressing human MerTK, and M2c-differentiated human macrophages, respectively.

FIG. 2 sets forth data showing reduced efferocytosis by phagocytic cells by various bispecific anti-MerTK:anti-PDL1 antibodies of the present disclosure.

FIG. 3 sets forth data showing pAKT/tAKT levels in cells treated with various bispecific anti-MerTK:anti-PDL1 antibodies of the present disclosure.

FIG. 4 sets forth data showing fold-change in pAKT activity in cells treated with various bispecific anti-MerTK:anti-PDL1 of the present disclosure in the absence of MerTK ligand huGas6.

FIG. 5 sets forth data showing reduced tumor volume in vivo in animals treated with a bivalent anti-PDL1 antibody compared to that observed in animals treated with a combination of a bivalent anti-PDL1 antibody and either bivalent anti-MerTK antibody MTK-16.2 or anti-MerTK antibody MTK-33.11.

FIG. 6 sets forth data showing changes in tumor volume in individual mice administered control antibody, bivalent anti-PDL1 antibody, and a combination of bivalent anti-PDL1 antibody and either bivalent anti-MerTK antibody MTK-16.2 or bivalent anti-MerTK antibody MTK-33.11

### DETAILED DESCRIPTION OF THE PRESENT DISCLOSURE

The present disclosure relates to anti-MerTK antibodies (e.g., monoclonal antibodies); methods of making and using such antibodies; pharmaceutical compositions comprising such antibodies; nucleic acids encoding such antibodies; and host cells comprising nucleic acids encoding such antibodies.

The techniques and procedures described or referenced herein are generally well understood and commonly employed using conventional methodology by those skilled in the art, such as, for example, the widely utilized methodologies such as those described in Sambrook et al. Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003); Monoclonal Antibodies: A Practical Approach (P. Shepherd and C. Dean, eds., Oxford University Press, 2000).

### I. Definitions

The terms “MerTK” or “MerTK polypeptide” or “MerTK protein” are used interchangeably herein refer herein to any native MerTK from any vertebrate source, including mammals such as primates (e.g., humans and cynomolgus monkeys (cynos)) and rodents (e.g., mice and rats), unless otherwise indicated. MerTK is also referred to as c-mer, MER, Proto-oncogene c-Mer, Receptor Tyrosine Kinase MerTK, Tyrosine-protein Kinase Mer, STK Kinase, RP38, and MGC133349. In some embodiments, the term encompasses both wild-type sequences and naturally occurring variant sequences, e.g., splice variants or allelic variants. In some embodiments, the term encompasses “full-length,” unprocessed MerTK as well as any form of MerTK that results from processing in the cell. In some embodiments, the MerTK is human MerTK. As used herein, the term “human MerTK” refers to a polypeptide with the amino acid sequence of SEQ ID NO:1.

The terms “anti-MerTK antibody,” an “antibody that binds to MerTK,” and “antibody that specifically binds MerTK” refer to an antibody that is capable of binding MerTK with sufficient affinity such that the antibody is useful as a diagnostic and/or therapeutic agent in targeting MerTK. In one embodiment, the extent of binding of an anti-MerTK antibody to an unrelated, non-MerTK polypeptide is less than about 10% of the binding of the antibody to MerTK as measured, e.g., by a radioimmunoassay (RIA). In certain embodiments, an antibody that binds to MerTK has a dissociation constant (KD) of <1 μM, <100 nM, <10 nM, <1 nM, <0.1 nM, <0.01 nM, or <0.001 nM (e.g., 10⁻⁸ M or less, e.g. from 10⁻⁸ M to 10⁻¹³ M, e.g., from 10⁻⁹ M to 10⁻¹³ M). In certain embodiments, an anti-MerTK antibody binds to an epitope of MerTK that is conserved among MerTK from different species.

With regard to the binding of an antibody to a target molecule, the term “specific binding” or “specifically binds” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a KD for the target of about any of 10⁻⁴ M or lower, 10⁻⁵ M or lower, 10⁻⁶ M or lower, 10⁻⁷ M or lower, 10⁻⁸ M or lower, 10⁻⁹ M or lower, 10⁻¹⁰ M or lower, 10⁻¹¹ M or lower, 10⁻¹² M or lower or a KD in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and KD values are inversely related. A high affinity for an antigen is measured by a low KD value. In one embodiment, the term “specific binding” refers to binding where a molecule binds to a particular polypeptide or epitope on a particular polypeptide without substantially binding to any other polypeptide or polypeptide epitope.

The term “immunoglobulin” (Ig) is used interchangeably with “antibody” herein. The term “antibody” herein is used in the broadest sense and specially covers monoclonal antibodies, polyclonal antibodies, multispecific antibodies (e.g., bispecific antibodies) including those formed from at least two intact antibodies, and antigen-binding antibody fragments so long as they exhibit the desired biological activity.

“Native antibodies” are usually heterotetrameric glycoproteins of about 150,000 Daltons, composed of two identical light (“L”) chains and two identical heavy (“H”) chains. Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, C T, 1994, page 71 and Chapter 6.

The light chain from any vertebrate species can be assigned to one of two clearly distinct types, called kappa (“κ”) and lambda (“λ”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ε”), gamma (“γ”), and mu (“μ”), respectively. The γ and α classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three-dimensional configurations of different classes of immunoglobulins are well known and described generally in, for example, Abbas et al., Cellular and Molecular Immunology, 4^{th} ed. (W.B. Saunders Co., 2000).

The “variable region” or “variable domain” of an antibody, such as an anti-MerTK antibody of the present disclosure, refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

The term “variable” refers to the fact that certain segments of the variable domains differ extensively in sequence among antibodies, such as anti-MerTK antibodies of the present disclosure. The variable domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Immunological Interest, Fifth Edition, National Institute of Health, Bethesda, MD (1991)). The constant domains are not involved directly in the binding of antibody to an antigen, but exhibit various effector functions, such as participation of the antibody in antibody-dependent-cellular toxicity.

The term “monoclonal antibody” as used herein refers to an antibody, such as a monoclonal anti-MerTK antibody of the present disclosure, obtained from a population of substantially homogeneous antibodies, i.e., the individual antibodies comprising the population are identical except for possible naturally occurring mutations and/or post-translation modifications (e.g., isomerizations, amidations, etc.) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, but not limited to one or more of the following methods, immunization methods of animals including, but not limited to rats, mice, rabbits, guinea pigs, hamsters and/or chickens with one or more of DNA(s), virus-like particles, polypeptide(s), and/or cell(s), the hybridoma methods, B-cell cloning methods, recombinant DNA methods, and technologies for producing human or human-like antibodies in animals that have parts or all of the human immunoglobulin loci or genes encoding human immunoglobulin sequences.

The terms “full-length antibody,” “intact antibody” or “whole antibody” are used interchangeably to refer to an antibody, such as an anti-MerTK antibody, in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

The terms “monovalent antibody” or “monoarm antibody” refers to an antibody having a single antigen-binding domain that is specific to a target antigen (i.e., the antibody comprises no more than one antigen-binding domain). In some embodiments, a single antigen-binding domain comprises a single variable region heavy chain polypeptide and a single variable region light chain polypeptide. An antibody that is “monovalent” for a target comprises no more than one antigen-binding domain for that target.

An “antibody fragment” refers to a molecule other than an intact antibody that comprises a portion of an intact antibody that binds the antigen to which the intact antibody binds. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10):1057-1062 (1995)); single-chain antibody molecules and multispecific antibodies formed from antibody fragments.

As used herein, the terms “antigen-binding domain,” “antigen-binding region,” “antigen-binding site,” and similar terms refer to the portion of antibody molecules which comprises the amino acid residues that confer on the antibody molecule its specificity for the antigen (e.g., the hypervariable regions (HVR)).

Papain digestion of antibodies, such as anti-MerTK antibodies of the present disclosure, produces two identical antigen-binding fragments, called “Fab” fragments, and a residual “Fc” fragment, a designation reflecting the ability to crystallize readily. The Fab fragment consists of an entire light chain along with the variable region domain of the heavy chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both heavy chains held together by disulfides. The effector functions of antibodies are determined by sequences in the Fc region, the region which is also recognized by Fc receptors (FcR) found on certain types of cells.

“Functional fragments” of antibodies, such as anti-MerTK antibodies of the present disclosure, comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments with short linkers (about 5-10 residues) between the V_{H} and V_{L} domains such that inter-chain but not intra-chain pairing of the variable domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains.

As used herein, a “chimeric antibody” refers to an antibody (immunoglobulin), such as a chimeric anti-MerTK antibody of the present disclosure, in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity. Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

“Humanized” forms of non-human (e.g., murine) antibodies, such as humanized forms of anti-MerTK antibodies of the present disclosure, are chimeric antibodies comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” is one that possesses an amino-acid sequence corresponding to that of an antibody, such as an anti-MerTK antibody of the present disclosure, produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries and yeast-display libraries. Human antibodies can be prepared by administering the antigen to a transgenic animal that has been modified to produce such antibodies in response to antigenic challenge, but whose endogenous loci have been disabled, e.g., immunized xenomice as well as generated via a human B-cell hybridoma technology.

The term “hypervariable region,” “HVR,” or “HV,” when used herein refers to the regions of an antibody-variable domain, such as that of an anti-MerTK antibody of the present disclosure, that are hypervariable in sequence and/or form structurally defined loops. Generally, antibodies comprise six HVRs; three in the V_{H} (H1, H2, H3), and three in the V_{L} (L1, L2, L3). In native antibodies, H3 and L3 display the most diversity of the six HVRs, and H3 in particular is believed to play a unique role in conferring fine specificity to antibodies. Naturally occurring camelid antibodies consisting of a heavy chain only are functional and stable in the absence of light chain.

A number of HVR delineations are in use and are encompassed herein. In some embodiments, the HVRs may be Kabat complementarity-determining regions (CDRs) based on sequence variability and are the most commonly used (Kabat et al., supra). In some embodiments, the HVRs may be Chothia CDRs. Chothia refers instead to the location of the structural loops (Chothia and Lesk J. Mol. Biol. 196:901-917 (1987)). In some embodiments, the HVRs may be AbM HVRs. The AbM HVRs represent a compromise between the Kabat CDRs and Chothia structural loops, and are used by Oxford Molecular's AbM antibody-modeling software. In some embodiments, the HVRs may be “contact” HVRs. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable-domain residues are numbered according to Kabat et al., supra, for each of these extended-HVR definitions.

“Framework” or “FR” residues are those variable-domain residues other than the HVR residues as herein defined.

An “acceptor human framework” as used herein is a framework comprising the amino acid sequence of a V_{L} or V_{H} framework derived from a human immunoglobulin framework or a human consensus framework. An acceptor human framework “derived from” a human immunoglobulin framework or a human consensus framework may comprise the same amino acid sequence thereof, or it may comprise pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less. Where pre-existing amino acid changes are present in a VH, preferable those changes occur at only three, two, or one of positions 71H, 73H and 78H; for instance, the amino acid residues at those positions may by 71A, 73T and/or 78A. In one embodiment, the VL acceptor human framework is identical in sequence to the VL human immunoglobulin framework sequence or human consensus framework sequence.

A “human consensus framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, MD (1991). Examples include for the V_{L}, the subgroup may be subgroup kappa I, kappa II, kappa III or kappa IV as in Kabat et al., supra. Additionally, for the V_{H}, the subgroup may be subgroup I, subgroup II, or subgroup III as in Kabat et al., supra.

An “amino-acid modification” at a specified position, e.g., of an anti-MerTK antibody of the present disclosure, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

“Fv” is the minimum antibody fragment which comprises a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the V_{H} and V_{L} domains, which enables the sFv to form the desired structure for antigen binding.

Antibody “effector functions” refer to those biological activities attributable to the Fc region (a native sequence Fc region or amino acid sequence variant Fc region) of an antibody, and vary with the antibody isotype.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the present disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least 80% homology with a native sequence Fc region and/or with an Fc region of a parent polypeptide, and most preferably at least 90% homology therewith, more preferably at least 95% homology therewith.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. Other FcRs, including those to be identified in the future, are encompassed by the term “FcR” herein. FcRs can also increase the serum half-life of antibodies.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full-length of the sequences being compared.

The term “compete” when used in the context of antibodies that compete for the same epitope or overlapping epitopes means competition between antibody as determined by an assay in which the antibody being tested prevents or inhibits (e.g., reduces) specific binding of a reference molecule (e.g., a ligand, or a reference antibody) to a common antigen (e.g., MerTK or a fragment thereof). Numerous types of competitive binding assays can be used to determine if antibody competes with another, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabeled test antibody and a labeled reference antibody. Competitive inhibition is measured by determining the amount of label bound to the solid surface or cells in the presence of the test antibody. Usually the test antibody is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur. Usually, when a competing antibody is present in excess, it will inhibit (e.g., reduce) specific binding of a reference antibody to a common antigen by at least 20%, 30%, 40%, 50%, 60%, 70%, 80%, 85%, 90%, 95%, 97.5%, and/or near 100%.

As used herein, an “interaction” between a MerTK polypeptide and a second polypeptide encompasses, without limitation, protein-protein interaction, a physical interaction, a chemical interaction, binding, covalent binding, and ionic binding. As used herein, an antibody “inhibits interaction” between two polypeptides when the antibody disrupts, reduces, or completely eliminates an interaction between the two polypeptides. An antibody of the present disclosure, thereof, “inhibits interaction” between two polypeptides when the antibody thereof binds to one of the two polypeptides. In some embodiments, the interaction can be inhibited by at least any of 20%, 30%, 40%, 50%, 60%, 70%, 80%, 85%, 90%, 95%, 97.5%, and/or near 100%.

The term “epitope” includes any determinant capable of being bound by an antibody. An epitope is a region of an antigen that is bound by an antibody that targets that antigen, and when the antigen is a polypeptide, includes specific amino acids that directly contact the antibody. Most often, epitopes reside on polypeptides, but in some instances, can reside on other kinds of molecules, such as nucleic acids. Epitope determinants can include chemically active surface groupings of molecules such as amino acids, sugar side chains, phosphoryl or sulfonyl groups, and can have specific three dimensional structural characteristics, and/or specific charge characteristics. Generally, antibodies specific for a particular target antigen will preferentially recognize an epitope on the target antigen in a complex mixture of polypeptides and/or macromolecules.

An “isolated” antibody, such as an isolated anti-MerTK antibody of the present disclosure, is one that has been identified, separated and/or recovered from a component of its production environment (e.g., naturally or recombinantly). Preferably, the isolated antibody is free of association with all other contaminant components from its production environment. Contaminant components from its production environment, such as those resulting from recombinant transfected cells, are materials that would typically interfere with research, diagnostic or therapeutic uses for the antibody, and may include enzymes, hormones, and other proteinaceous or non-proteinaceous solutes. In preferred embodiments, the antibody will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. Isolated antibody includes the antibody in situ within recombinant T-cells since at least one component of the antibody's natural environment will not be present. Ordinarily, however, an isolated polypeptide or antibody will be prepared by at least one purification step.

An “isolated” nucleic acid molecule encoding an antibody, such as an anti-MerTK antibody of the present disclosure, is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

“Carriers” as used herein include pharmaceutically acceptable carriers, excipients, or stabilizers that are nontoxic to the cell or mammal being exposed thereto at the dosages and concentrations employed.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

An “effective amount” refers to at least an amount effective, at dosages and for periods of time necessary, to achieve the desired therapeutic result. An effective amount can be provided in one or more administrations. An effective amount is also one in which any toxic or detrimental effects of the treatment are outweighed by the therapeutically beneficial effects. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount of drug, compound, or pharmaceutical composition is an amount sufficient to accomplish therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

An “individual” for purposes of treatment refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. In some embodiments, the individual is human.

As used herein, administration “in conjunction” or “in combination” with another compound or composition includes simultaneous administration and/or administration at different times. Administration in conjunction or in combination also encompasses administration as a co-formulation or administration as separate compositions, including at different dosing frequencies or intervals, and using the same route of administration or different routes of administration. In some embodiments, administration in conjunction is administration as a part of the same treatment regimen.

The term “about” as used herein refers to the usual error range for the respective value readily known to the skilled person in this technical field. Reference to “about” a value or parameter herein includes (and describes) embodiments that are directed to that value or parameter per se.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

It is understood that aspect and embodiments of the present disclosure described herein include “comprising,” “consisting,” and “consisting essentially of” aspects and embodiments.

### II. Bispecific Anti-MerTK:Anti-PDL1 Antibodies

Provided herein are bispecific anti-MerTK:anti-PDL1 antibodies. Antibodies provided herein are useful, e.g., for the treatment of MerTK associated disorders.

In one aspect, the present disclosure provides isolated (e.g., monoclonal) antibodies that bind to an epitope within a MerTK protein or polypeptide of the present disclosure. MerTK proteins or polypeptides of the present disclosure include, without limitation, a mammalian MerTK protein or polypeptide, human MerTK protein or polypeptide, mouse (murine) MerTK protein or polypeptide, and cynomolgus MerTK protein or polypeptide. MerTK proteins and polypeptides of the present disclosure include naturally occurring variants of MerTK. In some embodiments, MerTK proteins and polypeptides of the present disclosure are membrane bound. In some embodiments, MerTK proteins and polypeptides of the present disclosure are a soluble extracellular domain of MerTK.

In some embodiments, MerTK is expressed in a cell. In some embodiments, MerTK is expressed in phagocytic cells, including without limitation, macrophages and dendritic cells. In some embodiments, MerTK is expressed in monocytes, natural killer cells, natural killer T cells, microglia, endothelial cells, megakaryocytes, and platelets. In some embodiments, high levels of MerTK expression are also found in ovary, prostate, testis, lung, retina, and kidney. Additionally, MerTK displays ectopic or overexpression in numerous cancers (Linger et al, 2008, Adv Cancer Res, 100:35-83).

### III. MerTK Binding Partners

MerTK proteins of the present disclosure interact with (e.g., bind) one or more ligands or binding partners, including, without limitation, Protein S (ProS or ProS1), Growth arrest specific gene 6 (Gas6), Tubby, Tubby-like protein 1 (TULP-1), and Galectin-3. Anti-MerTK antibodies of the present disclosure can affect the interaction of MerTK with one or more of its various ligands and binding partners. In particular, parental mouse anti-MerTK antibody MTK-16 and parental mouse anti-MerTK antibody MTK-33 are effective at blocking the binding of both Gas6 ligand and ProS ligand to MerTK, as disclosed in International Patent Application Serial No. PCT/US2020/064640) (WO 2021/119508).

### IV. pAKT

AKT activity is a downstream target of Gas6 binding to MerTK, Axl, or Tyro-3 receptors. For example, the binding of MerTK ligand Gas6 to MerTK induces AKT phosphorylation (pAKT) (see, e.g., Angelillo-Scherrer et al, 2008, J Clin Invest, 118:583-596; Moody et al, 2016, Int J Cancer, 139:1340-1349). Bispecific anti-MerTK:anti-PDL1 antibodies of the present disclosure were effective at reducing Gas6-mediated phospho-AKT (pAKT) activity in human macrophages (e.g., M2c-differentiated human macrophages) in a dose-dependent manner. Accordingly, anti-MerTK:anti-PDL1 antibodies of the present disclosure were effective at reducing Gas6-mediated MerTK signaling as evidenced by reduction of pAKT levels. In some embodiments, a bispecific anti-MerTK:anti-PDL1 antibody of the present disclosure inhibits or reduces Gas6-mediated pAKT activity in vitro.

The relative effectiveness of a bispecific anti-MerTK:anti-PDL1 antibody at reducing pAKT activity in a cell can be determined by measuring the IC50 values. IC50 values for reduction of Gas6-mediated pAKT activity can be determined using methods known by one of skill in the art, such as that described herein in Example 13 below.

### V. Efferocytosis

Efferocytosis refers to phagocytic clearance of dying or apoptotic cells. Efferocytosis can be accomplished by professional phagocytes (e.g., macrophages, dendritic cells, microglia), non-professional phagocytes (e.g., epithelial cells, fibroblasts, retinal pigment epithelial cells), or specialized phagocytes. (Elliott et al, 2017, J Immunol, 198:1387-1394). Efferocytosis leads to the removal of dead or dying cells before their membrane integrity is breached and their cellular contents leak into the surrounding tissue, thus preventing exposure of tissue to toxic enzymes, oxidants, and other intracellular components.

Apoptotic cells expose a variety of molecules on their cell surface (“eat-me” signals) that are recognized by receptors on phagocytic cells. One such “eat me” signaling molecules is phosphatidylserine (PtdSer), which is normally confined to the inner leaflet of the cell membrane. During apoptosis, PtdSer is exposed to the outer leaflet of the cell membrane. MerTK ligands ProS and Gas6 contain gamma-carboxylated glutamic acid residues near their N-terminal domains; gamma-carboxylation of the glutamic acid domain enables binding to phosphatidylserine. Gas6 or ProS bind to PtdSer on apoptotic cells and simultaneously bind MerTK on phagocytes. Such ligand engagement with MerTK activates efferocytosis.

As shown in Example 12 below, bispecific anti-MerTK:anti-PDL1 antibodies of the present disclosure were effective at reducing efferocytosis activity by phagocytic cells.

Accordingly, in some embodiments, a bispecific anti-MerTK:anti-PDL1 antibody of the present disclosure reduces efferocytosis by phagocytic cells. In some embodiments, a bispecific anti-MerTK:anti-PDL1 antibody of the present disclosure reduces efferocytosis by macrophages. In some embodiments, a bispecific anti-MerTK:anti-PDL1 antibody of the present disclosure reduces efferocytosis by dendritic cells. In some embodiments, a bispecific anti-MerTK:anti-PDL1 antibody of the present disclosure reduces efferocytosis by bone marrow-derived macrophages. Reduction of efferocytosis can be determined using standard methods known to one of skill in the art, such as described herein in Example 12.

In some embodiments, a bispecific anti-MerTK:anti-PDL1 antibody of the present disclosure reduces efferocytosis by a phagocytic cell (e.g., a human macrophage) with an IC50 in the range of about 0.13 nM to about 30 nM, as assessed by methods described herein in Example 12. In some embodiments, a bispecific anti-MerTK:anti-PDL1 antibody of the present disclosure reduces efferocytosis by a phagocytic cell with an IC50 value in the range of approximately 4.4 nM to approximately 15.68 nM.

Blocking efferocytosis drives M1-like macrophage polarization, resulting in increased production of pro-inflammatory cytokines (e.g., TNF, IFN, IL-12) and recruitment of cytotoxic cells, such as CD8+ T cells and natural killer cells, that mediate anti-tumor immunity. By reducing efferocytosis by phagocytic cells, anti-MerTK:anti-PDL1 antibodies of the present disclosure are thus effective at increasing M1-like macrophage polarization and at increasing production, expression, and/or secretion of pro-inflammatory cytokines/chemokines, including TNF, IFN, IL-6, IL-1, IL-12, chemokine (C-X-C motif) ligand 1 (CXCL-1, KC), monocyte chemoattractant protein-1 (MCP1, CCL2), macrophage inflammatory protein-1-alpha (MIP-1α, CCL3), and/or macrophage inflammatory protein-1-beta (MIP-1β, CCL4).

A link between efferocytosis and cancer progression has been described. For example, blockade of efferocytosis using Annexin V, which blocks PtdSer from interacting with the efferocytosis machinery of phagocytes, sufficiently reduces tumor progression and metastasis (Stach et al, 2000, Cell Death Diff, 7:911; Bondanza et al, 2004, J Exp Med, 200:1157; Werfel and Cook, 2018, Sem Immunopathology, 40:545-554). Further, MerTK correlates with poor prognosis and survival in numerous human cancers, as does its PtdSer bridging ligand Gas6 (Graham et al, 2014, Nat Rev Cancer, 14:769; Linger et al, 2010, Expert Opin Ther Targets, 14:1073-1090; Wang et al, 2013, Oncogene, 32:872; Jansen et al, 2011, J Proteome Res, 11:728-735; Tworkoski et al, 2011, Mol Cancer Res, p.molcanres-0512; Graham et al, 2006, Clin Cancer Res, 12:2662-2669; Keating et al, 2006, Oncogene, 25:6092). Accordingly, anti-MerTK antibodies of the present disclosure, which reduce efferocytosis by phagocytic cells, are thus effective at reducing tumor progression and metastasis.

Cynomolgus studies indicated that in some instances, bivalent anti-MerTK antibodies that block binding to both Gas6 and ProS (e.g., anti-MerTK antibody MTK-16) showed less in vivo toxicity (e.g., weight loss) compared to that observed in cynomolgus monkeys administered a bivalent anti-MerTK antibody that blocks ProS binding to MerTK but does not block binding of Gas6 to MerTK. Accordingly, in some embodiments, a bispecific anti-MerTK:anti-PDL1 antibody of the present disclosure that is monovalent for MerTK and blocks binding of both Gas6 and ProS to MerTK may display less systemic in vivo toxicity compared to an anti-MerTK antibody of the present disclosure that blocks binding of ProS to MerTK but does not block binding of Gas6 to MerTK. Anti-MerTK antibody MTK-16 and anti-MerTK antibody MTK-33 (which both block binding of both Gas6 and ProS to MerTK) bind to the Ig1 domain of MerTK protein, while anti-MerTK antibody MTK-15 (which blocks binding of ProS to MerTK, but which does not block binding of Gas6 to MerTK) bind to both the Ig2 and the FN1 domain of MerTK protein (see International Patent Application Serial No. PCT/US2020/064640 (WO 2021/119508). Accordingly, in some embodiments, a bispecific anti-MerTK:anti-PDL1 antibody that binds to the Ig1 domain of MerTK (e.g., bispecific anti-MerTK:anti-PDL1 antibody MTK-16, MTK-16.2, MTK-33, and MTK-33.11) may display less systemic in vivo toxicity than an anti-MerTK antibody that binds to both the Ig2 and FN1 domains of MerTK. Additionally, in some embodiments, a bispecific anti-MerTK:anti-PDL1 antibody that binds to the Ig1 domain of MerTK (e.g., bispecific anti-MerTK:anti-PDL1 antibody MTK-16, MTK-16.2, MTK-33, and MTK-33.11) and displays reduced pAKT activity (in part, due to its monovalent configuration), may display less systemic in vivo toxicity than a bivalent anti-MerTK antibody that binds to both the Ig2 and FN1 domains of MerTK.

### Bispecific Antibody Configurations

Many different formats and uses of bispecific antibodies are known in the art (reviewed in, e.g., Kontermann; Drug Discov Today, 2015 July; 20(7):838-47; MAbs, 2012 March-April; 4(2): 182-97). A bispecific antibody according to the present invention is not limited to any particular bispecific format or method of producing it. Accordingly, bispecific anti-MerTK:anti-PDL1 antibodies of the present disclosure can include various configurations of a bispecific antibody having a first antigen-binding domain that binds to human MerTK and a second antigen-binding domain that binds to PDL1.

Examples of bispecific antibody molecules which may be used in the present disclosure comprise (i) a single antibody that has two arms comprising different antigen-binding domains; (ii) a single chain antibody that has specificity to two different epitopes, e.g., via two scFvs linked in tandem by an extra peptide linker; (iii) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig.TM.) Molecule, In: Antibody Engineering, Springer Berlin Heidelberg (2010)); (iv) a chemically-linked bispecific (Fab′)2 fragment; (v) a Tandab, which is a fusion of two single chain diabodies resulting in a tetravalent bispecific antibody that has two binding sites for each of the target antigens; (vi) a flexibody, which is a combination of scFvs with a diabody resulting in a multivalent molecule; (vii) a so-called “dock and lock” molecule, based on the “dimerization and docking domain” in Protein Kinase A, which, when applied to Fabs, can yield a trivalent bispecific binding protein consisting of two identical Fab fragments linked to a different Fab fragment; (viii) a so-called Scorpion molecule, comprising, e.g., two scFvs fused to both termini of a human Fab-arm; and (ix) a diabody.

In some aspects, a dimerized Fc region of a bispecific anti-MerTK:anti-PDL1 antibody of the present disclosure is formed by Fc regions that contain amino acid mutations, substitutions, additions, or deletions to promote heterodimerization in which different polypeptides comprising different Fc regions can dimerize to yield a heterodimer configuration. In one aspect, a bispecific antibody of the present disclosure comprises a first Fc sequence comprising a first CH3 region, and a second Fc sequence comprising a second CH3 region, wherein the sequences of the first and second CH3 regions are different and are such that the heterodimeric interaction between said first and second CH3 regions is stronger than each of the homodimeric interactions of said first and second CH3 regions.

Methods to promote heterodimerization of Fc regions include amino acid deletions, additions, or substitutions of the amino acid sequence of the Fc region, such as by including a set of “knob-into-hole” deletions, additions, or substitutions or including amino acid deletions, additions, or substitutions to effect electrostatic steering of the Fc to favor attractive interactions among different polypeptide chains. Methods for promoting heterodimerization of complementary Fc polypeptides have been previously described in, for example, Ridgway et al, 1996, Protein Eng, 9:617-621; Merchant et al, 1998, Nature Biotechnol, 16:677-681; Moore et al, 2011, MAbs, 3:546-557; Von Kreudenstein et al, 2013, 5:646-654; Gunasekaran et al, 2010, J Biol Chem, 285:19637-19464; Leaver-Fay et al, 2016, Structure, 24:641-651; Ha et al, 2016, Frontiers in Immunology, 7:1; Davis et al, 2010, Protein Eng Des Sel, 23:195-202; WO1996/027011; WO1998/050431; WO2006/028936; WO2009/089004; WO2011/143545; WO2014/067011; WO2012/058768; WO2018/027025; US2014/0363426; US2015/0307628; US2018/0016354; US2015/0239991; US2017/0058054; U.S. Pat. Nos. 5,731,168; 7,183,076; 9,701,759; 9,605,084; 9,650,446; 8,216,805; 8,765,412; and 8,258,268.

For example, in some embodiments, complementary Fc polypeptides of an Fc heterodimer include a mutation to alter charge polarity across the Fc dimer interface such that co-expression of electrostatically matched Fc regions support favorable attractive interactions, thereby promoting desired Fc heterodimer formation; whereas unfavorable repulsive charge interactions suppress unwanted Fc homodimer formation (Guneskaran et al, 2010, J Biol Chem, 285:19637-19646). When co-expressed in a cell, association between the polypeptide chains is possible but the chains do not substantially self-associate due to charge repulsion.

Additionally, complementary Fc polypeptides of an Fc heterodimer include “knob-into-hole” configurations to promote heterodimerization of two Fc polypeptides. “Knob-into-hole” technology is described in e.g. U.S. Pat. Nos. 5,731,168; 7,695,936; 8,216,805; 8,765,412; Ridgway et al., Prot Eng 9, 617-621 (1996); and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)). Thus, in such configurations, a first Fc region polypeptide comprises amino acid modifications to form the “knob” and a second Fc region polypeptide comprises amino acid modifications to form the “hole” thus forming an Fc heterodimer comprising complementary Fc polypeptides.

Exemplary paired amino acid modifications of complementary Fc polypeptides of an Fc heterodimeric configuration are set forth below in Table A (EU numbering).

TABLE A

| First Fc polypeptide | Second Fc polypeptide |
|---|---|
| T366Y | Y407T |
| T366W | T366S/L368W/Y407V |
| T366W | T366S/L368A/Y407V |
| T366W/S354C | T366S/L368A/Y407V/Y349C |
| T350V/L351Y/F405A/Y407V | T350V/T366L/K392L/T394W |
| K360D/D399M/Y407A | E345R/Q347R/T366V/K409V |
| K409D/K392D | D399K/E356K |
| K360E/K409W | Q347R/D399V/F405T |
| L360E/K409W/Y349C | Q347R/D399V/F405T/S354C |
| K370E/K409W | E357N/D399V/F405T |

In one particular embodiment of a “knob-into-hole” configuration, a first Fc polypeptide of an Fc heterodimeric configuration comprises a T366Y amino acid substitution, and a second Fc polypeptide of the Fc heterodimeric configuration comprises a Y407T amino acid substitution (EU numbering). In another particular embodiment of a “knob-into-hole” configuration, a first Fc polypeptide of an Fc heterodimeric configuration comprises a T366W amino acid substitution, and a second Fc polypeptide of the Fc heterodimeric configuration comprises a T366S, L368A, and Y407V amino acid substitutions (EU numbering).

### A. Exemplary Antibodies and Certain Other Antibody Embodiments

The present disclosure provides bispecific anti-MerTK:anti-PDL1 antibodies comprising a first antigen-binding domain that binds to human MerTK and a second antigen-binding domain that binds to PDL1.

In some embodiments, a bispecific anti-MerTK:anti-PDL1 antibody of the present disclosure comprises an anti-MerTK antibody heavy chain comprising a wildtype IgG Fc amino acid sequence and an anti-PDL1 antibody heavy chain comprising a wildtype IgG Fc amino acid sequence. In some embodiments, a bispecific anti-MerTK:anti-PDL1 antibody of the present disclosure comprises an anti-MerTK antibody heavy chain comprising a wildtype IgG1 Fc amino acid sequence and an anti-PDL1 antibody heavy chain comprising a wildtype IgG1 Fc amino acid sequence. In some embodiments, a bispecific anti-MerTK:anti-PDL1 antibody of the present disclosure comprises an anti-MerTK antibody heavy chain comprising an IgG Fc region having LALAPS amino acid substitutions an anti-PDL1 antibody heavy chain comprising an IgG Fc region having LALAPS amino acid substitutions. In some embodiments, a bispecific anti-MerTK:anti-PDL1 antibody of the present disclosure comprises an anti-MerTK antibody heavy chain comprising an IgG1 Fc region having LALAPS amino acid substitutions an anti-PDL1 antibody heavy chain comprising an IgG1 Fc region having LALAPS amino acid substitutions. In some embodiments, a bispecific anti-MerTK:anti-PDL1 antibody of the present disclosure comprises an anti-MerTK antibody heavy chain comprising an IgG Fc region having NSLF amino acid substitutions an anti-PDL1 antibody heavy chain comprising an IgG Fc region having NSLF amino acid substitutions. In some embodiments, a bispecific anti-MerTK:anti-PDL1 antibody of the present disclosure comprises an anti-MerTK antibody heavy chain comprising an IgG1 Fc region having NSLF amino acid substitutions an anti-PDL1 antibody heavy chain comprising an IgG1 Fc region having NSLF amino acid substitutions. In some embodiments, a bispecific anti-MerTK:anti-PDL1 antibody of the present disclosure comprises an anti-MerTK antibody heavy chain comprising a wildtype IgG4 Fc amino acid sequence and an anti-PDL1 antibody heavy chain comprising a wildtype IgG4 Fc amino acid sequence. In some embodiments, a bispecific anti-MerTK:anti-PDL1 antibody of the present disclosure comprises an anti-MerTK antibody heavy chain comprising an IgG4 Fc amino acid sequence having any of the amino acid substitutions described above and further comprising an S228P amino acid substitution, and an anti-PDL1 antibody heavy chain comprising an IgG4 Fc amino acid sequence having any of the amino acid substitutions described above and further comprising an S228P amino acid substitution.

In some embodiments, an anti-MerTK:anti-PDL1 antibody of the present disclosure comprises an anti-MerTK antibody heavy chain comprising “knob” amino acid substitutions in the Fc region, and an anti-PDL1 antibody heavy chain comprising “hole” amino acid substitutions in the Fc region. In some embodiments, an anti-MerTK:anti-PDL1 antibody of the present disclosure comprises an anti-MerTK antibody heavy chain comprising “hole” amino acid substitutions in the Fc region, and an anti-PDL1 antibody heavy chain comprising “knob” amino acid substitutions in the Fc region.

In some embodiments, a bispecific anti-MerTK:anti-PDL1 antibody of the present disclosure comprises a first antigen-binding domain that binds to human MerTK comprising a heavy chain variable region comprising the amino acid sequence of SEQ ID NO:9 and a light chain variable region comprising the amino acid sequence of SEQ ID NO:10, and a second antigen-binding domain that binds to PDL1 comprising a heavy chain variable region comprising the amino acid sequence of SEQ ID NO:52 and a light chain variable region comprising the amino acid sequence of SEQ ID NO:53.

In some embodiments, a bispecific anti-MerTK:anti-PDL1 antibody of the present disclosure comprises a first antigen-binding domain that binds to human MerTK comprising a heavy chain comprising the amino acid sequence of SEQ ID NO: 17 and a light chain comprising the amino acid sequence of SEQ ID NO:21, and a second antigen-binding domain that binds to PDL1 comprising a heavy chain comprising the amino acid sequence of SEQ ID NO:27 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising the amino acid sequence of SEQ ID NO: 18 and a light chain comprising the amino acid sequence of SEQ ID NO:21, and a second antigen-binding domain that binds to PDL1 comprising a heavy chain comprising the amino acid sequence of SEQ ID NO:28 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising the amino acid sequence of SEQ ID NO: 19 and a light chain comprising the amino acid sequence of SEQ ID NO:21, and a second antigen-binding domain that binds to PDL1 comprising a heavy chain comprising the amino acid sequence of SEQ ID NO:29 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising the amino acid sequence of SEQ ID NO:20 and a light chain comprising the amino acid sequence of SEQ ID NO:21, and a second antigen-binding domain that binds to PDL1 comprising a heavy chain comprising the amino acid sequence of SEQ ID NO:30 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising the amino acid sequence of SEQ ID NO:32 and a light chain comprising the amino acid sequence of SEQ ID NO:21, and a second antigen-binding domain that binds to PDL1 comprising a heavy chain comprising the amino acid sequence of SEQ ID NO:42 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising the amino acid sequence of SEQ ID NO:33 and a light chain comprising the amino acid sequence of SEQ ID NO:21, and a second antigen-binding domain that binds to PDL1 comprising a heavy chain comprising the amino acid sequence of SEQ ID NO:43 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising the amino acid sequence of SEQ ID NO:34 and a light chain comprising the amino acid sequence of SEQ ID NO:21, and a second antigen-binding domain that binds to PDL1 comprising a heavy chain comprising the amino acid sequence of SEQ ID NO:44 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising the amino acid sequence of SEQ ID NO:35 and a light chain comprising the amino acid sequence of SEQ ID NO:21, and a second antigen-binding domain that binds to PDL1 comprising a heavy chain comprising the amino acid sequence of SEQ ID NO:45 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising the amino acid sequence of SEQ ID NO:36 and a light chain comprising the amino acid sequence of SEQ ID NO:21, and a second antigen-binding domain that binds to PDL1 comprising a heavy chain comprising the amino acid sequence of SEQ ID NO:46 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain variable region comprising the amino acid sequence of SEQ ID NO:11 and a light chain variable region comprising the amino acid sequence of SEQ ID NO: 12, and a second antigen-binding domain that binds to PDL1 comprising a heavy chain variable region comprising the amino acid sequence of SEQ ID NO:52 and a light chain variable region comprising the amino acid sequence of SEQ ID NO:53.

In some embodiments, a bispecific anti-MerTK:anti-PDL1 antibody of the present disclosure comprises a first antigen-binding domain that binds to human MerTK comprising a heavy chain comprising the amino acid sequence of SEQ ID NO:22 and a light chain comprising the amino acid sequence of SEQ ID NO:26, and a second antigen-binding domain that binds to PDL1 comprising a heavy chain comprising the amino acid sequence of SEQ ID NO:27 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising the amino acid sequence of SEQ ID NO:23 and a light chain comprising the amino acid sequence of SEQ ID NO:26, and a second antigen-binding domain that binds to PDL1 comprising a heavy chain comprising the amino acid sequence of SEQ ID NO:28 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising the amino acid sequence of SEQ ID NO:24 and a light chain comprising the amino acid sequence of SEQ ID NO:26, and a second antigen-binding domain that binds to PDL1 comprising a heavy chain comprising the amino acid sequence of SEQ ID NO:29 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising the amino acid sequence of SEQ ID NO:25 and a light chain comprising the amino acid sequence of SEQ ID NO:26, and a second antigen-binding domain that binds to PDL1 comprising a heavy chain comprising the amino acid sequence of SEQ ID NO:30 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising the amino acid sequence of SEQ ID NO:37 and a light chain comprising the amino acid sequence of SEQ ID NO:26, and a second antigen-binding domain that binds to PDL1 comprising a heavy chain comprising the amino acid sequence of SEQ ID NO:42 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising the amino acid sequence of SEQ ID NO:38 and a light chain comprising the amino acid sequence of SEQ ID NO:26, and a second antigen-binding domain that binds to PDL1 comprising a heavy chain comprising the amino acid sequence of SEQ ID NO:43 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising the amino acid sequence of SEQ ID NO:39 and a light chain comprising the amino acid sequence of SEQ ID NO:26, and a second antigen-binding domain that binds to PDL1 comprising a heavy chain comprising the amino acid sequence of SEQ ID NO:44 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising the amino acid sequence of SEQ ID NO:40 and a light chain comprising the amino acid sequence of SEQ ID NO:26, and a second antigen-binding domain that binds to PDL1 comprising a heavy chain comprising the amino acid sequence of SEQ ID NO:45 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising the amino acid sequence of SEQ ID NO:41 and a light chain comprising the amino acid sequence of SEQ ID NO:26, and a second antigen-binding domain that binds to PDL1 comprising a heavy chain comprising the amino acid sequence of SEQ ID NO:46 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising amino acids 1-449 of SEQ ID NO:17 and a light chain comprising the amino acid sequence of SEQ ID NO:21, and a second antigen-binding domain that binds to PDL1 comprising a heavy chain comprising amino acids 1-447 of SEQ ID NO:27 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising amino acids 1-449 of SEQ ID NO: 18 and a light chain comprising the amino acid sequence of SEQ ID NO:21, and a second antigen-binding domain that binds to PDL1 comprising a heavy chain comprising amino acids 1-447 of SEQ ID NO:28 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising amino acids 1-449 of SEQ ID NO: 19 and a light chain comprising the amino acid sequence of SEQ ID NO:21, and a second antigen-binding domain that binds to PDL1 comprising a heavy chain comprising amino acids 1-447 of SEQ ID NO:29 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising amino acids 1-446 of SEQ ID NO:20 and a light chain comprising the amino acid sequence of SEQ ID NO:21, and a second antigen-binding domain that binds to PDL1 comprising a heavy chain comprising amino acids 1-444 of SEQ ID NO:30 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising amino acids 1-453 of SEQ ID NO:32 and a light chain comprising the amino acid sequence of SEQ ID NO:21, and a second antigen-binding domain that binds to PDL1 comprising a heavy chain comprising amino acids 1-447 of SEQ ID NO:42 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising amino acids 1-449 of SEQ ID NO:33 and a light chain comprising the amino acid sequence of SEQ ID NO:21, and a second antigen-binding domain that binds to PDL1 comprising a heavy chain comprising amino acids 1-447 of SEQ ID NO:43 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising amino acids 1-449 of SEQ ID NO:34 and a light chain comprising the amino acid sequence of SEQ ID NO:21, and a second antigen-binding domain that binds to PDL1 comprising a heavy chain comprising amino acids 1-447 of SEQ ID NO:44 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising amino acids 1-446 of SEQ ID NO:35 and a light chain comprising the amino acid sequence of SEQ ID NO:21, and a second antigen-binding domain that binds to PDL1 comprising a heavy chain comprising amino acids 1-444 of SEQ ID NO:45 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising amino acids 1-446 of SEQ ID NO:36 and a light chain comprising the amino acid sequence of SEQ ID NO:21, and a second antigen-binding domain that binds to PDL1 comprising a heavy chain comprising amino acids 1-444 of SEQ ID NO:46 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising amino acids 1-448 of SEQ ID NO:22 and a light chain comprising the amino acid sequence of SEQ ID NO:26, and a second antigen-binding domain that binds to PDL1 comprising a heavy chain comprising amino acids 1-447 of SEQ ID NO:27 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising amino acids 1-448 of SEQ ID NO:23 and a light chain comprising the amino acid sequence of SEQ ID NO:26, and a second antigen-binding domain that binds to PDL1 comprising a heavy chain comprising amino acids 1-447 of SEQ ID NO:28 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising amino acids 1-448 of SEQ ID NO:24 and a light chain comprising the amino acid sequence of SEQ ID NO:26, and a second antigen-binding domain that binds to PDL1 comprising a heavy chain comprising amino acids 1-447 of SEQ ID NO:29 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising amino acids 1-445 of SEQ ID NO:25 and a light chain comprising the amino acid sequence of SEQ ID NO:26, and a second antigen-binding domain that binds to PDL1 comprising a heavy chain comprising amino acids 1-444 of SEQ ID NO:30 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising amino acids 1-448 of SEQ ID NO:37 and a light chain comprising the amino acid sequence of SEQ ID NO:26, and a second antigen-binding domain that binds to PDL1 comprising a heavy chain comprising amino acids 1-447 of SEQ ID NO:42 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising amino acids 1-447 of SEQ ID NO:38 and a light chain comprising the amino acid sequence of SEQ ID NO:26, and a second antigen-binding domain that binds to PDL1 comprising a heavy chain comprising amino acids 1-447 of SEQ ID NO:43 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising amino acids 1-448 of SEQ ID NO:39 and a light chain comprising the amino acid sequence of SEQ ID NO:26, and a second antigen-binding domain that binds to PDL1 comprising a heavy chain comprising amino acids 1-447 of SEQ ID NO:44 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising amino acids 1-445 of SEQ ID NO:40 and a light chain comprising the amino acid sequence of SEQ ID NO:26, and a second antigen-binding domain that binds to PDL1 comprising a heavy chain comprising amino acids 1-444 of SEQ ID NO:45 and a light chain comprising the amino acid sequence of SEQ ID NO:31.

In some embodiments, a bispecific anti-MerTK:anti-PDL1 antibody of the present disclosure comprises a first antigen-binding domain that binds to human MerTK comprising a heavy chain comprising amino acids 1-445 of SEQ ID NO:41 and a light chain comprising the amino acid sequence of SEQ ID NO:26, and a second antigen-binding domain that binds to PDL1 comprising a heavy chain comprising amino acids 1-444 of SEQ ID NO:46 and a light chain comprising the amino acid sequence of SEQ ID NO:31.

In some embodiments, a bispecific anti-MerTK:anti-PDL1 antibody according to any of the above embodiments may incorporate any of the features, singly or in combination, as described in Sections 1-7 below:

### (1) Anti-MerTK Antibody Binding Affinity

In some embodiments of any of the antibodies provided herein, the antibody has a dissociation constant (KD) of <1 μM, <100 nM, <10 nM, <1 nM, <0.1 nM, <0.01 nM, or <0.001 nM (e.g., 10⁻⁸ M or less, e.g., from 10⁻⁸ M to 10⁻¹³ M, e.g., from 10⁻⁹ M to 10⁻¹³ M). Dissociation constants may be determined through any analytical technique, including any biochemical or biophysical technique such as ELISA, surface plasmon resonance (SPR), bio-layer interferometry (see, e.g., Octet System by ForteBio), isothermal titration calorimetry (ITC), differential scanning calorimetry (DSC), circular dichroism (CD), stopped-flow analysis, and colorimetric or fluorescent protein melting analyses. In one embodiment, Kd is measured by a radiolabeled antigen binding assay (RIA). In some embodiment, an RIA is performed with the Fab version of an antibody of interest and its antigen, for example as described in Chen et al. J. Mol. Biol. 293:865-881(1999)). In some embodiments, KD is measured using a BIACORE surface plasmon resonance assay, for example, an assay using a BIACORE-2000 or a BIACORE-3000 (BIAcore, Inc., Piscataway, NJ) is performed at 25° C. with immobilized antigen CM5 chips at ˜10 response units (RU). In some embodiments, the KD is determined using a monovalent antibody (e.g., a Fab) or a full-length antibody. In some embodiments, the K_{D} is determined using a full-length antibody in a monovalent form.

In some embodiments, an anti-MerTK antibody of the present disclosure binds to human MerTK, wherein the K_{D} of binding to human MerTK is from about 1.4 nM to about 81 nM. In some embodiments, an anti-MerTK antibody binds to cyno MerTK, wherein the K_{D} of binding to cyno MerTK is from about 1.6 nM to about 107 nM. In some embodiments, an anti-MerTK antibody of the present disclosure binds to murine MerTK, wherein the K_{D} of binding to murine MerTK is from about 30 nM to about 186 nM.

### (2) Antibody Fragments

In some embodiments of any of the antibodies provided herein, the antibody is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP404097; WO 1993/01161; Hudson et al. Nat. Med. 9:129-134 (2003). Triabodies and tetrabodies are also described in Hudson et al. Nat. Med. 9:129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

### (3) Chimeric and Humanized Antibodies

In some embodiments of any of the antibodies provided herein, the antibody is a chimeric antibody. Certain chimeric antibodies are described, e.g., in U.S. Pat. No. 4,816,567. In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some embodiments of any of the antibodies provided herein, the antibody is a humanized antibody. Typically, a non-human antibody is humanized to reduce immunogenicity to humans, while retaining the specificity and affinity of the parental non-human antibody. In certain embodiments, a humanized antibody is substantially non-immunogenic in humans. In certain embodiments, a humanized antibody has substantially the same affinity for a target as an antibody from another species from which the humanized antibody is derived. See, e.g., U.S. Pat. Nos. 5,530,101, 5,693,761; 5,693,762; and 5,585,089. In certain embodiments, amino acids of an antibody variable domain that can be modified without diminishing the native affinity of the antigen-binding domain while reducing its immunogenicity are identified. See, e.g., U.S. Pat. Nos. 5,766,886 and 5,869,619. Generally, a humanized antibody comprises one or more variable domains in which HVRs (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), for example, to restore or improve antibody specificity or affinity.

Humanized antibodies and methods of making them are reviewed, for example, in Almagro et al. Front. Biosci. 13:161 9-1633 (2008), and are further described, e.g., in U.S. Pat. Nos. 5,821,337, 7,527,791, 6,982,321, and 7,087,409. Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA 89:4285 (1992); and Presta et al., J. Immunol. 151:2623 (1993)); human mature (somatically mutated) framework regions or human germline framework regions (see, e.g., Almagro and Fransson Front. Biosci. 13:1619-1633 (2008)); and framework regions derived from screening FR libraries (see, e.g., Baca et al. J. Biol. Chem. 272:10678-10684 (1997) and Rosok et al. J. Biol. Chem. 271:22611-22618 (1996)).

### (4) Human Antibodies

In some embodiments of any of the antibodies provided herein, the antibody is a human antibody. Human antibodies can be produced using various techniques known in the art. Human antibodies are described generally in van Dijk et al. Curr. Opin. Pharmacol. 5:368-74 (2001) and Lonberg Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. One can engineer mouse strains deficient in mouse antibody production with large fragments of the human Ig loci in anticipation that such mice would produce human antibodies in the absence of mouse antibodies. Large human Ig fragments can preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains can yield high affinity fully human antibodies against any antigen of interest, including human antigens. Using the hybridoma technology, antigen-specific human MAbs with the desired specificity can be produced and selected. Certain exemplary methods are described in U.S. Pat. No. 5,545,807, EP 546073, and EP 546073. See also, for example, U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology. Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol. 133:3001 (1984) and Boerner et al. J. Immunol. 147:86 (1991)). Human antibodies generated via human B-cell hybridoma technology are also described in Li et al. Proc. Natl. Acad. Sci. USA, 1 03:3557-3562 (2006). Additional methods include those described, for example, in U.S. Pat. No. 7,189,826 (describing production of monoclonal human IgM antibodies from hybridoma cell lines). Human hybridoma technology (Trioma technology) is also described in Vollmers et al. Histology and Histopathology 20(3): 927-937 (2005) and Vollmers et al. Methods and Findings in Experimental and Clinical Pharmacology 27(3): 185-91 (2005). Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

In some embodiments of any of the antibodies provided herein, the antibody is a human antibody isolated by in vitro methods and/or screening combinatorial libraries for antibodies with the desired activity or activities. Suitable examples include but are not limited to phage display (CAT, Morphosys, Dyax, Biosite/Medarex, Xoma, Symphogen, Alexion (formerly Proliferon), Affimed) ribosome display (CAT), yeast display (Adimab), and the like. In certain phage display methods, repertoires of VH and VL genes are separately cloned by polymerase chain reaction (PCR) and recombined randomly in phage libraries, which can then be screened for antigen-binding phage as described in Winter et al. Ann. Rev. Immunol. 12: 433-455 (1994). For example, a variety of methods are known in the art for generating phage display libraries and screening such libraries for antibodies possessing the desired binding characteristics. See also Sidhu et al. J. Mol. Biol. 338(2): 299-310, 2004; Lee et al. J. Mol. Biol. 340(5): 1073-1093, 2004; Fellouse Proc. Natl. Acad. Sci. USA 101(34): 12467-12472 (2004); and Lee et al. J. Immunol. Methods 284(-2): 1 19-132 (2004). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al. EMBO J. 12: 725-734 (1993). Finally, naive libraries can also be made synthetically by cloning unrearranged V-gene segments from stem cells, and using PCR primers comprising random sequence to encode the highly variable HVR3 regions and to accomplish rearrangement in vitro, as described by Hoogenboom et al. J. Mol. Biol., 227: 381-388, 1992. Patent publications describing human antibody phage libraries include, for example: U.S. Pat. No. 5,750,373, and US Patent Publication Nos. 2007/0292936 and 2009/0002360. Antibodies isolated from human antibody libraries are considered human antibodies or human antibody fragments herein.

### (5) Constant Regions Including Fc Regions

In some embodiments of any of the antibodies provided herein, the antibody comprises an Fc. In some embodiments, the Fc is a human IgG1, IgG2, IgG3, and/or IgG4 isotype. In some embodiments, the antibody is of the IgG class, the IgM class, or the IgA class.

In certain embodiments of any of the antibodies provided herein, the antibody has an IgG2 isotype. In some embodiments, the antibody contains a human IgG2 constant region. In some embodiments, the human IgG2 constant region includes an Fc region. In some embodiments, the antibody induces the one or more MerTK activities or independently of binding to an Fc receptor. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has an IgG1 isotype. In some embodiments, the antibody contains a mouse IgG1 constant region. In some embodiments, the antibody contains a human IgG1 constant region. In some embodiments, the human IgG1 constant region includes an Fc region. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has an IgG4 isotype. In some embodiments, the antibody contains a human IgG4 constant region. In some embodiments, the human IgG4 constant region includes an Fc region. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has a hybrid IgG2/4 isotype. In some embodiments, the antibody includes an amino acid sequence comprising amino acids 118 to 260 according to EU numbering of human IgG2 and amino acids 261-447 according to EU numbering of human IgG4 (WO 1997/11971; WO 2007/106585).

In some embodiments, the Fc region increases clustering without activating complement as compared to a corresponding antibody comprising an Fc region that does not comprise the amino acid substitutions. In some embodiments, the antibody induces one or more activities of a target specifically bound by the antibody. In some embodiments, the antibody binds to MerTK.

It may also be desirable to modify an anti-MerTK antibody of the present disclosure to modify effector function and/or to increase serum half-life of the antibody. For example, the Fc receptor binding site on the constant region may be modified or mutated to remove or reduce binding affinity to certain Fc receptors, such as FcγRI, FcγRII, and/or FcγRIII to reduce Antibody-dependent cell-mediated cytotoxicity. In some embodiments, the effector function is impaired by removing N-glycosylation of the Fc region (e.g., in the CH2 domain of IgG) of the antibody. In some embodiments, the effector function is impaired by modifying regions such as 233-236, 297, and/or 327-331 of human IgG as described in WO 99/58572 and Armour et al. Molecular Immunology 40: 585-593 (2003); Reddy et al. J. Immunology 164:1925-1933 (2000). In other embodiments, it may also be desirable to modify an anti-MerTK antibody of the present disclosure to modify effector function to increase finding selectivity toward the ITIM-containing FcgRIIb (CD32b) to increase clustering of MerTK antibodies on adjacent cells without activating humoral responses including Antibody-dependent cell-mediated cytotoxicity and antibody-dependent cellular phagocytosis.

To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule. Other amino acid sequence modifications.

### (6) Antibody Variants

In some embodiments of any of the antibodies provided herein, amino acid sequence variants of the antibodies are contemplated. For example, it may be desirable to improve the binding affinity and/or other biological properties of the antibody.

### (i) Substitution, Insertion, and Deletion Variants

In some embodiments of any of the antibodies provided herein, antibody variants having one or more amino acid substitutions are provided. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody.

TABLE A
Amino Acid Substitutions

| Original | | |
|---|---|---|
| Residue | Exemplary Substitutions | Preferred Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Tyr |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro; and
(6) aromatic: Trp, Tyr, Phe.

For example, non-conservative substitutions can involve the exchange of a member of one of these classes for a member from another class. Such substituted residues can be introduced, for example, into regions of a human antibody that are homologous with non-human antibodies, or into the non-homologous regions of the molecule.

In making changes to the polypeptide or antibody described herein, according to certain embodiments, the hydropathic index of amino acids can be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al. J. Mol. Biol., 157:105-131 (1982). It is known that certain amino acids can be substituted for other amino acids having a similar hydropathic index or score and still retain a similar biological activity. In making changes based upon the hydropathic index, in certain embodiments, the substitution of amino acids whose hydropathic indices are within +2 is included. In certain embodiments, those which are within +1 are included, and in certain embodiments, those within +0.5 are included.

It is also understood in the art that the substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0±1); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within +2 is included, in certain embodiments, those which are within +1 are included, and in certain embodiments, those within +0.5 are included. One can also identify epitopes from primary amino acid sequences on the basis of hydrophilicity. These regions are also referred to as “epitopic core regions”.

In certain embodiments of the variant VH and VL sequences provided above, each HVR is unaltered.

Amino acid sequence insertions include amino- and/or carboxyl-terminal fusions ranging in length from one residue to polypeptides comprising a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

Any cysteine residue outside the HVRs and not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment, such as an Fv fragment).

### (ii) Glycosylation Variants

In some embodiments of any of the antibodies provided herein, the antibody is altered to increase or decrease the extent to which the antibody is glycosylated. Addition or deletion of glycosylation sites to an antibody may be conveniently accomplished by altering the amino acid sequence such that one or more glycosylation sites is created or removed.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Where the antibody comprises an Fc region, the carbohydrate attached thereto may be altered. Native antibodies produced by mammalian cells typically comprise a branched, biantennary oligosaccharide that is generally attached by an N-linkage to Asn297 according to Kabat numbering of the CH2 domain of the Fc region. The oligosaccharide may include various carbohydrates, for example, mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the disclosure may be made in order to create antibody variants with certain improved properties.

In one embodiment, antibody variants are provided having a carbohydrate structure that lacks fucose attached (directly or indirectly) to an Fc region. See, e.g., US Patent Publication Nos. 2003/0157108 and 2004/0093621. Examples of publications related to “defucosylated” or “fucose-deficient” antibody variants include: US 2003/0157108; US 2003/0115614; US 2002/0164328; US 2004/0093621; US 2004/0132140; US 2004/0110704; US 2004/0110282; US 2004/0109865; Okazaki et al. J. Mol. Biol. 336:1239-1249 (2004); Yamane-Ohnuki et al. Biotech. Bioeng. 87:614 (2004). Examples of cell lines capable of producing defucosylated antibodies include Led 3 CHO cells deficient in protein fucosylation (Ripka et al. Arch. Biochem. Biophys. 249:533-545 (1986); US 2003/0157108), and knockout cell lines, such as alpha-1,6-fucosyltransferase gene, FUT8, knockout CHO cells (see, e.g., Yamane-Ohnuki et al. Biotech. Bioeng. 87: 614 (2004) and Kanda et al. Biotechnol. Bioeng. 94(4):680-688 (2006)).

(iii) Modified Constant Regions

In some embodiments of any of the antibodies provided herein, the antibody Fc is an antibody Fc isotypes and/or modifications. In some embodiments, the antibody Fc isotype and/or modification is capable of binding to Fc gamma receptor.

In some embodiments of any of the antibodies provided herein, the modified antibody Fc is an IgG1 modified Fc. In some embodiments, the IgG1 modified Fc comprises one or more modifications. For example, in some embodiments, the IgG1 modified Fc comprises one or more amino acid substitutions (e.g., relative to a wild-type Fc region of the same isotype). In some embodiments, the one or more amino acid substitutions are selected from N297A (Bolt S et al. (1993) Eur J Immunol 23:403-411), D265A (Shields et al. (2001) R. J. Biol. Chem. 276, 6591-6604), L234A, L235A (Hutchins et al. (1995) Proc Natl Acad Sci USA, 92:11980-11984; Alegre et al., (1994) Transplantation 57:1537-1543. 31; Xu et al., (2000) (′ell Immunol, 200:16-26), G237A (Alegre et al. (1994) Transplantation 57:1537-1543. 31; Xu et al. (2000) Cell Immunol, 200:16-26), C226S, C229S, E233P, L234V, L234F, L235E (McEarchern et al., (2007) Blood, 109:1185-1192), P331S (Sazinsky et al., (2008) Proc Natl Acad Sci USA 2008, 105:20167-20172), S267E, L328F, A330L, M252Y, S254T, and/or T256E, where the amino acid position is according to the EU numbering convention.

In some embodiments of any of the IgG1 modified Fc, the Fc comprises N297A mutation according to EU numbering. In some embodiments of any of the IgG1 modified Fc, the Fc comprises D265A and N297A mutations according to EU numbering. In some embodiments of any of the IgG1 modified Fc, the Fc comprises D270A mutations according to EU numbering. In some embodiments, the IgG1 modified Fc comprises L234A and L235A mutations according to EU numbering. In some embodiments of any of the IgG1 modified Fc, the Fc comprises L234A and G237A mutations according to EU numbering. In some embodiments of any of the IgG1 modified Fc, the Fc comprises L234A, L235A and G237A mutations according to EU numbering. In some embodiments of any of the IgG1 modified Fc, the Fc comprises one or more (including all) of P238D, L328E, E233, G237D, H268D, P271G and A330R mutations according to EU numbering. In some embodiments of any of the IgG1 modified Fc, the Fc comprises one or more of S267E/L328F mutations according to EU numbering. In some embodiments of any of the IgG1 modified Fc, the Fc comprises P238D, L328E, E233D, G237D, H268D, P271G and A330R mutations according to EU numbering. In some embodiments of any of the IgG1 modified Fc, the Fc comprises P238D, L328E, G237D, H268D, P271G and A330R mutations according to EU numbering. In some embodiments of any of the IgG1 modified Fc, the Fc comprises P238D, S267E, L328E, E233D, G237D, H268D, P271G and A330R mutations according to EU numbering. In some embodiments of any of the IgG1 modified Fc, the Fc comprises P238D, S267E, L328E, G237D, H268D, P271G and A330R mutations according to EU numbering. In some embodiments of any of the IgG1 modified Fc, the Fc comprises C226S, C229S, E233P, L234V, and L235A mutations according to EU numbering. In some embodiments of any of the IgG1 modified Fc, the Fc comprises L234F, L235E, and P331S mutations according to EU numbering. In some embodiments of any of the IgG1 modified Fc, the Fc comprises S267E and L328F mutations according to EU numbering. In some embodiments of any of the IgG1 modified Fc, the Fc comprises N325S and L328F mutations according to EU numbering. In some embodiments of any of the IgG1 modified Fc, the Fc comprises S267E mutations according to EU numbering. In some embodiments of any of the IgG1 modified Fc, the Fc comprises a substitute of the constant heavy 1 (CH1) and hinge region of IgG1 with CH1 and hinge region of IgG2 (amino acids 118-230 of IgG2 according to EU numbering) with a Kappa light chain.

In some embodiments of any of the IgG1 modified Fc, the Fc includes two or more amino acid substitutions that increase antibody clustering without activating complement as compared to a corresponding antibody having an Fc region that does not include the two or more amino acid substitutions. Accordingly, in some embodiments of any of the IgG1 modified Fc, the IgG1 modified Fc is an antibody comprising an Fc region, where the antibody comprises an amino acid substitution at position E430G and one or more amino acid substitutions in the Fc region at a residue position selected from: L234F, L235A, L235E, S267E, K322A, L328F, A330S, P331S, and any combination thereof according to EU numbering. In some embodiments, the IgG1 modified Fc comprises an amino acid substitution at positions E430G, L243A, L235A, and P331S according to EU numbering. In some embodiments, the IgG1 modified Fc comprises an amino acid substitution at positions E430G and P331S according to EU numbering. In some embodiments, the IgG1 modified Fc comprises an amino acid substitution at positions E430G and K322A according to EU numbering. In some embodiments, the IgG1 modified Fc comprises an amino acid substitution at positions E430G, A330S, and P331S according to EU numbering. In some embodiments, the IgG1 modified Fc comprises an amino acid substitution at positions E430G, K322A, A330S, and P331S according to EU numbering. In some embodiments, the IgG1 modified Fc comprises an amino acid substitution at positions E430G, K322A, and A330S according to EU numbering. In some embodiments, the IgG1 modified Fc comprises an amino acid substitution at positions E430G, K322A, and P331S according to EU numbering.

In some embodiments of any of the IgG1 modified Fc, the IgG1 modified Fc may further comprise herein may be combined with an A330L mutation (Lazar et al. Proc Natl Acad Sci USA, 103:4005-4010 (2006)), or one or more of L234F, L235E, and/or P331S mutations (Sazinsky et al. Proc Natl Acad Sci USA, 105:20167-20172 (2008)), according to the EU numbering convention, to eliminate complement activation. In some embodiments of any of the IgG1 modified Fc, the IgG1 modified Fc may further comprise one or more of A330L, A330S, L234F, L235E, and/or P331S according to EU numbering. In some embodiments of any of the IgG1 modified Fc, the IgG1 modified Fc may further comprise one or more mutations to enhance the antibody half-life in human serum (e.g., one or more (including all) of M252Y, S254T, and T256E mutations according to the EU numbering convention). In some embodiments of any of the IgG1 modified Fc, the IgG1 modified Fc may further comprise one or more of E430G, E430S, E430F, E430T, E345K, E345Q, E345R, E345Y, S440Y, and/or S440W according to EU numbering.

Other aspects of the present disclosure relate to antibodies having modified constant regions (i.e., Fc regions). An antibody dependent on binding to FcgR receptor to activate targeted receptors may lose its agonist activity if engineered to eliminate FcgR binding (see, e.g., Wilson et al. Cancer Cell 19:101-113 (2011); Armour at al. Immunology 40:585-593 (2003); and White et al. Cancer Cell 27:138-148 (2015)). As such, it is thought that an anti-MerTK antibody of the present disclosure with the correct epitope specificity can activate the target antigen, with minimal adverse effects, when the antibody has an Fc domain from a human IgG2 isotype (CH1 and hinge region) or another type of Fc domain that is capable of preferentially binding the inhibitory FcgRIIB r receptors, or a variation thereof.

In some embodiments of any of the antibodies provided herein, the modified antibody Fc is an IgG2 modified Fc. In some embodiments, the IgG2 modified Fc comprises one or more modifications. For example, in some embodiments, the IgG2 modified Fc comprises one or more amino acid substitutions (e.g., relative to a wild-type Fc region of the same isotype). In some embodiments of any of the IgG2 modified Fc, the one or more amino acid substitutions are selected from V234A (Alegre et al. Transplantation 57:1537-1543 (1994); Xu et al. Cell Immunol, 200:16-26 (2000)); G237A (Cole et al. Transplantation, 68:563-571 (1999)); H268Q, V309L, A330S, P331S (US 2007/0148167; Armour et al. Eur J Immunol 29: 2613-2624 (1999); Armour et al. The Haematology Journal 1(Suppl. 1):27 (2000); Armour et al. The Haematology Journal 1(Suppl.1):27 (2000)), C219S, and/or C220S (White et al. Cancer Cell 27, 138-148 (2015)); S267E, L328F (Chu et al. Mol Immunol, 45:3926-3933 (2008)); and M252Y, S254T, and/or T256E according to the EU numbering convention. In some embodiments of any of the IgG2 modified Fc, the Fc comprises an amino acid substitution at positions V234A and G237A according to EU numbering. In some embodiments of any of the IgG2 modified Fc, the Fc comprises an amino acid substitution at positions C219S or C220S according to EU numbering. In some embodiments of any of the IgG2 modified Fc, the Fc comprises an amino acid substitution at positions A330S and P331S according to EU numbering. In some embodiments of any of the IgG2 modified Fc, the Fc comprises an amino acid substitution at positions S267E and L328F according to EU numbering.

In some embodiments of any of the IgG2 modified Fc, the Fc comprises a C127S amino acid substitution according to the EU numbering convention (White et al., (2015) Cancer Cell 27, 138-148; Lightle et al. Protein Sci. 19:753-762 (2010); and WO 2008/079246). In some embodiments of any of the IgG2 modified Fc, the antibody has an IgG2 isotype with a Kappa light chain constant domain that comprises a C214S amino acid substitution according to the EU numbering convention (White et al. Cancer Cell 27:138-148 (2015); Lightle et al. Protein Sci. 19:753-762 (2010); and WO 2008/079246).

In some embodiments of any of the IgG2 modified Fc, the Fc comprises a C220S amino acid substitution according to the EU numbering convention. In some embodiments of any of the IgG2 modified Fc, the antibody has an IgG2 isotype with a Kappa light chain constant domain that comprises a C214S amino acid substitution according to the EU numbering convention.

In some embodiments of any of the IgG2 modified Fc, the Fc comprises a C219S amino acid substitution according to the EU numbering convention. In some embodiments of any of the IgG2 modified Fc, the antibody has an IgG2 isotype with a Kappa light chain constant domain that comprises a C214S amino acid substitution according to the EU numbering convention.

In some embodiments of any of the IgG2 modified Fc, the Fc includes an IgG2 isotype heavy chain constant domain 1(CH1) and hinge region (White et al. Cancer Cell 27:138-148 (2015)). In certain embodiments of any of the IgG2 modified Fc, the IgG2 isotype CH1 and hinge region comprise the amino acid sequence of 118-230 according to EU numbering. In some embodiments of any of the IgG2 modified Fc, the antibody Fc region comprises a S267E amino acid substitution, a L328F amino acid substitution, or both, and/or a N297A or N297Q amino acid substitution according to the EU numbering convention.

In some embodiments of any of the IgG2 modified Fc, the Fc further comprises one or more amino acid substitution at positions E430G, E430S, E430F, E430T, E345K, E345Q, E345R, E345Y, S440Y, and S440W according to EU numbering. In some embodiments of any of the IgG2 modified Fc, the Fc may further comprise one or more mutations to enhance the antibody half-life in human serum (e.g., one or more (including all) of M252Y, S254T, and T256E mutations according to the EU numbering convention). In some embodiments of any of the IgG2 modified Fc, the Fc may further comprise A330S and P331S.

In some embodiments of any of the IgG2 modified Fc, the Fc is an IgG2/4 hybrid Fc. In some embodiments, the IgG2/4 hybrid Fc comprises IgG2 aa 118 to 260 and IgG4 aa 261 to 447. In some embodiments of any IgG2 modified Fc, the Fc comprises one or more amino acid substitutions at positions H268Q, V309L, A330S, and P331S according to EU numbering.

In some embodiments of any of the IgG1 and/or IgG2 modified Fc, the Fc comprises one or more additional amino acid substitutions selected from A330L, L234F; L235E, or P331S according to EU numbering; and any combination thereof.

In certain embodiments of any of the IgG1 and/or IgG2 modified Fc, the Fc comprises one or more amino acid substitutions at a residue position selected from C127S, L234A, L234F, L235A, L235E, S267E, K322A, L328F, A330S, P331S, E345R, E430G, S440Y, and any combination thereof according to EU numbering. In some embodiments of any of the IgG1 and/or IgG2 modified Fc, the Fc comprises an amino acid substitution at positions E430G, L243A, L235A, and P331S according to EU numbering. In some embodiments of any of the IgG1 and/or IgG2 modified Fc, the Fc comprises an amino acid substitution at positions E430G and P331S according to EU numbering. In some embodiments of any of the IgG1 and/or IgG2 modified Fc, the Fc comprises an amino acid substitution at positions E430G and K322A according to EU numbering. In some embodiments of any of the IgG1 and/or IgG2 modified Fc, the Fc comprises an amino acid substitution at positions E430G, A330S, and P331S according to EU numbering. In some embodiments of any of the IgG1 and/or IgG2 modified Fc, the Fc comprises an amino acid substitution at positions E430G, K322A, A330S, and P331S according to EU numbering. In some embodiments of any of the IgG1 and/or IgG2 modified Fc, the Fc comprises an amino acid substitution at positions E430G, K322A, and A330S according to EU numbering. In some embodiments of any of the IgG1 and/or IgG2 modified Fc, the Fc comprises an amino acid substitution at positions E430G, K322A, and P331S according to EU numbering. In some embodiments of any of the IgG1 and/or IgG2 modified Fc, the Fc comprises an amino acid substitution at positions S267E and L328F according to EU numbering. In some embodiments of any of the IgG1 and/or IgG2 modified Fc, the Fc comprises an amino acid substitution at position C127S according to EU numbering. In some embodiments of any of the IgG1 and/or IgG2 modified Fc, the Fc comprises an amino acid substitution at positions E345R, E430G and S440Y according to EU numbering.

In some embodiments of any of the antibodies provided herein, the modified antibody Fc is an IgG4 modified Fc. In some embodiments, the IgG4 modified Fc comprises one or more modifications. For example, in some embodiments, the IgG4 modified Fc comprises one or more amino acid substitutions (e.g., relative to a wild-type Fc region of the same isotype). In some embodiments of any of the IgG4 modified Fc, the one or more amino acid substitutions are selected from L235A, G237A, S229P, L236E (Reddy et al. J Immunol 164:1925-1933(2000)), S267E, E318A, L328F, M252Y, S254T, and/or T256E according to the EU numbering convention. In some embodiments of any of the IgG4 modified Fc, the Fc may further comprise L235A, G237A, and E318A according to the EU numbering convention. In some embodiments of any of the IgG4 modified Fc, the Fc may further comprise S228P and L235E according to the EU numbering convention. In some embodiments of any of the IgG4 modified Fc, the IgG4 modified Fc may further comprise S267E and L328F according to the EU numbering convention.

In some embodiments of any of the IgG4 modified Fc, the IgG4 modified Fc comprises may be combined with an S228P mutation according to the EU numbering convention (Angal et al. Mol Immunol. 30:105-108 (1993)) and/or with one or more mutations described in (Peters et al. J Biol Chem. 287(29):24525-33 (2012)) to enhance antibody stabilization.

In some embodiments of any of the IgG4 modified Fc, the IgG4 modified Fc may further comprise one or more mutations to enhance the antibody half-life in human serum (e.g., one or more (including all) of M252Y, S254T, and T256E mutations according to the EU numbering convention).

In some embodiments of any of the IgG4 modified Fc, the Fc comprises L235E according to EU numbering. In certain embodiments of any of the IgG4 modified Fc, the Fc comprises one or more amino acid substitutions at a residue position selected from C127S, F234A, L235A, L235E, S267E, K322A, L328F, E345R, E430G, S440Y, and any combination thereof, according to EU numbering. In some embodiments of any of the IgG4 modified Fc, the Fc comprises an amino acid substitution at positions E430G, L243A, L235A, and P331S according to EU numbering. In some embodiments of any of the IgG4 modified Fc, the Fc comprises an amino acid substitution at positions E430G and P331S according to EU numbering. In some embodiments of any of the IgG4 modified Fc, the Fc comprises an amino acid substitution at positions E430G and K322A according to EU numbering. In some embodiments of any of the IgG4 modified Fc, the Fc comprises an amino acid substitution at position E430 according to EU numbering. In some embodiments of any of the IgG4 modified Fc, the Fc region comprises an amino acid substitution at positions E430G and K322A according to EU numbering. In some embodiments of any of the IgG4 modified Fc, the Fc comprises an amino acid substitution at positions S267E and L328F according to EU numbering. In some embodiments of any of the IgG4 modified Fc, the Fc comprises an amino acid substitution at position C127S according to EU numbering. In some embodiments of any of the IgG4 modified Fc, the Fc comprises an amino acid substitution at positions E345R, E430G and S440Y according to EU numbering.

### Other Antibody Modifications

In some embodiments of any of the antibodies, the antibody is a derivative. The term “derivative” refers to a molecule that includes a chemical modification other than an insertion, deletion, or substitution of amino acids (or nucleic acids). In certain embodiments, derivatives comprise covalent modifications, including, but not limited to, chemical bonding with polymers, lipids, or other organic or inorganic moieties. In certain embodiments, a chemically modified antigen-binding protein can have a greater circulating half-life than an antigen binding protein that is not chemically modified. In certain embodiments, a chemically modified antigen binding protein can have improved targeting capacity for desired cells, tissues, and/or organs. In some embodiments, a derivative antigen-binding protein is covalently modified to include one or more water soluble polymer attachments, including, but not limited to, polyethylene glycol, polyoxyethylene glycol, or polypropylene glycol. See, e.g., U.S. Pat. Nos. 4,640,835, 4,496,689, 4,301,144, 4,670,417, 4,791,192 and 4,179,337. In certain embodiments, a derivative antigen binding protein comprises one or more polymer, including, but not limited to, monomethoxy-polyethylene glycol, dextran, cellulose, copolymers of ethylene glycol/propylene glycol, carboxymethylcellulose, polyvinyl pyrrolidone, poly-1, 3-dioxolane, poly-1,3,6-trioxane, ethylene/maleic anhydride copolymer, polyaminoacids (either homopolymers or random copolymers), poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of such polymers.

In certain embodiments, a derivative is covalently modified with polyethylene glycol (PEG) subunits. In certain embodiments, one or more water-soluble polymer is bonded at one or more specific position, for example at the amino terminus, of a derivative. In certain embodiments, one or more water-soluble polymer is randomly attached to one or more side chains of a derivative. In certain embodiments, PEG is used to improve the therapeutic capacity for an antigen-binding protein. In certain embodiments, PEG is used to improve the therapeutic capacity for a humanized antibody. Certain such methods are discussed, for example, in U.S. Pat. No. 6,133,426, which is hereby incorporated by reference for any purpose.

Peptide analogs are commonly used in the pharmaceutical industry as non-peptide drugs with properties analogous to those of the template peptide. These types of non-peptide compound are termed “peptide mimetics” or “peptidomimetics.” Fauchere, J. Adv. Drug Res., 15:29 (1986); and Evans et al. J. Med. Chem., 30:1229 (1987), which are incorporated herein by reference for any purpose. Such compounds are often developed with the aid of computerized molecular modeling. Peptide mimetics that are structurally similar to therapeutically useful peptides can be used to produce a similar therapeutic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from: —CH₂NH—, —CH₂S—, —CH₂—CH₂—, —CH═CH-(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) can be used in certain embodiments to generate more stable peptides. In addition, constrained peptides comprising a consensus sequence or a substantially identical consensus sequence variation can be generated by methods known in the art (Rizo and Gierasch Ann. Rev. Biochem., 61:387 (1992), incorporated herein by reference for any purpose); for example, by adding internal cysteine residues capable of forming intramolecular disulfide bridges which cyclize the peptide.

Drug conjugation involves coupling of a biological active cytotoxic (anticancer) payload or drug to an antibody that specifically targets a certain tumor marker (e.g. a polypeptide that, ideally, is only to be found in or on tumor cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cancer. Due to this targeting, ideally the drug has lower side effects and gives a wider therapeutic window than other chemotherapeutic agents. Technics to conjugate antibodies are disclosed are known in the art (see, e.g., Jane de Lartigue OncLive Jul. 5, 2012; ADC Review on antibody-drug conjugates; and Ducry et al. Bioconjugate Chemistry 21 (1):5-13 (2010).

### VI. Nucleic Acids, Vectors, and Host Cells

Anti-MerTK antibodies of the present disclosure may be produced using recombinant methods and compositions, e.g., as described in U.S. Pat. No. 4,816,567. In some embodiments, isolated nucleic acids having a nucleotide sequence encoding any of the anti-MerTK antibodies of the present disclosure are provided. Such nucleic acids may encode an amino acid sequence comprising the V_{L} and/or an amino acid sequence comprising the V_{H} of the anti-MerTK antibody (e.g., the light and/or heavy chains of the antibody). In some embodiments, one or more vectors (e.g., expression vectors) comprising such nucleic acids are provided. In some embodiments, a host cell comprising such nucleic acid is also provided. In some embodiments, the host cell comprises (e.g., has been transduced with): (1) a vector comprising a nucleic acid that encodes an amino acid sequence comprising the V_{L} of the antibody and an amino acid sequence comprising the V_{H} of the antibody, or (2) a first vector comprising a nucleic acid that encodes an amino acid sequence comprising the V_{L} of the antibody and a second vector comprising a nucleic acid that encodes an amino acid sequence comprising the V_{H} of the antibody.

In some embodiments, the host cell comprises (e.g., has been transduced with): (1) a nucleic acid that encodes an amino acid sequence comprising a light chain of an antibody, wherein the light chain comprises a V_{L} and (2) a nucleic acid that encodes an amino acid sequence comprising a heavy chain of an antibody, wherein the heavy chain comprises a V_{H}, wherein the V_{L} and the V_{H} form an antigen-binding domain that binds to MerTK. In some embodiments, the host cell comprises (e.g., has been transduced with): (1) a nucleic acid that encodes an amino acid sequence comprising a light chain of an antibody, wherein the light chain comprises a V_{L}, (2) a nucleic acid that encodes an amino acid sequence comprising a heavy chain of an antibody, wherein the heavy chain comprises a V_{H}, and (3) a nucleic acid that encodes a fragment of a heavy chain, wherein the heavy chain not comprise a V_{H} (e.g., a fragment of a heavy chain comprising a CH₂ and a CH₃ domain), wherein the V_{L} and the V_{H} form an antigen-binding domain that binds to MerTK. The nucleic acids can be within the same vector or can be in different vectors.

In some embodiments, the host cell is eukaryotic, e.g., a Chinese Hamster Ovary (CHO) cell or lymphoid cell (e.g., Y0, NS0, Sp20 cell). Host cells of the present disclosure also include, without limitation, isolated cells, in vitro cultured cells, and ex vivo cultured cells.

Methods of making an anti-MerTK antibody of the present disclosure are provided. In some embodiments, the method includes culturing a host cell of the present disclosure comprising a nucleic acid encoding the anti-MerTK antibody, under conditions suitable for expression of the antibody. In some embodiments, the antibody is subsequently recovered from the host cell (or host cell culture medium).

For recombinant production of an anti-MerTK antibody of the present disclosure, a nucleic acid encoding the anti-MerTK antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable vectors comprising a nucleic acid sequence encoding any of the anti-MerTK antibodies of the present disclosure, or cell-surface expressed fragments or polypeptides thereof polypeptides (including antibodies) described herein include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones comprising the vector. Suitable examples include plasmids and bacterial viruses, e.g., pUC18, pUC19, Bluescript (e.g., pBS SK+) and its derivatives, mpl8, mpl9, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Suitable host cells for cloning or expression of antibody-encoding vectors include prokaryotic or eukaryotic cells. For example, anti-MerTK antibodies of the present disclosure may be produced in bacteria, in particular when glycosylation and Fc effector function are not needed. For expression of antibody fragments and polypeptides in bacteria (e.g., U.S. Pat. Nos. 5,648,237, 5,789,199, and 5,840,523. After expression, the antibody may be isolated from the bacterial cell paste in a soluble fraction and can be further purified.

In addition to prokaryotes, eukaryotic microorganisms, such as filamentous fungi or yeast, are also suitable cloning or expression hosts for antibody-encoding vectors, including fungi and yeast strains whose glycosylation pathways have been “humanized,” resulting in the production of an antibody with a partially or fully human glycosylation pattern (e.g., Gerngross Nat. Biotech. 22:1409-1414 (2004); and Li et al. Nat. Biotech. 24:210-215 (2006)).

Suitable host cells for the expression of glycosylated antibody can also be derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts (e.g., U.S. Pat. Nos. 5,959,177, 6,040,498, 6,420,548, 7,125,978, and 6,417,429, describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al. J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al. Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al. Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, NJ), pp. 255-268 (2003).

### VII. Pharmaceutical Compositions/Formulations

Provided herein are pharmaceutical compositions and/or pharmaceutical formulations comprising the bispecific anti-MerTK:anti-PDL1 antibodies of the present disclosure and a pharmaceutically acceptable carrier.

In some embodiments, pharmaceutically acceptable carrier preferably are nontoxic to recipients at the dosages and concentrations employed. The pharmaceutical compositions and/or pharmaceutical formulations to be used for in vivo administration can be sterile. This is readily accomplished by filtration through, e.g., sterile filtration membranes

Pharmaceutical compositions and/or pharmaceutical formulations provided herein are useful as a medicament, e.g., for treating cancer.

### VIII. Therapeutic Uses

As disclosed herein, bispecific anti-MerTK:anti-PDL1 antibodies of the present disclosure may be used for treating diseases and disorders. In some embodiments, the present disclosure provides methods for treating an individual having cancer comprising administering to the individual a therapeutically effective amount of a bispecific anti-MerTK:anti-PDL1 antibody of the present disclosure.

Ectopic or expression of MerTK has been observed in various tumors; overexpression and activation of MerTK has been implicated in lymphoid leukemia, lymphoma, adenoma, melanoma, gastric, prostate, and breast cancers; and MerTK overexpression has been associated with metastasis. (Schlegel et al, 2013, J Clin Invest, 123:2257-2267; Tworkoski et al, 2013, Pigment Cell Melanoma, 26:527-541; Yi et al, 2017, Oncotarget, 8:96656-96667; Linger et al, 2013, Blood, 122: 1599-1609; Lee-Sherick et al, 2013, Oncogene, 32:5359-5368; Brandao et al, 2013, Blood Cancer, 3:e101; Xie et al, 2015, Oncotarget, 6:9206-9219; Shi et al, 2018, J Hematology & Oncology, 11:43). Accordingly, modulating the activity of MerTK with a bispecific anti-MerTK:anti-PDL1 antibody of the present disclosure is an effective means of treating cancer.

In certain aspects, provided herein are methods for treating cancer in a subject in need thereof, the method comprising administering to the subject a bispecific anti-MerTK:anti-PDL1 antibody of the present disclosure, or a pharmaceutical composition comprising a bispecific anti-MerTK:anti-PDL1 antibody of the present disclosure. In some embodiments, a method is provided for treating cancer in a subject in need thereof, the method comprising administering to the subject a bispecific anti-MerTK:anti-PDL1 antibody of the present disclosure, wherein the bispecific anti-MerTK:anti-PDL1 antibody reduces efferocytosis by phagocytic cells.

In some embodiments, the cancer is selected from sarcoma, bladder cancer, breast cancer, colon cancer, endometrial cancer, kidney cancer, renal cancer, leukemia, lung cancer, non-small cell lung cancer, melanoma, lymphoma, pancreatic cancer, prostate cancer, ovarian cancer, stomach cancer, thyroid cancer, cancer of the uterus, liver cancer, cervical cancer, testicular cancer, squamous cell carcinoma, glioma, glioblastoma, adenoma, and neuroblastoma. In some embodiments, the cancer is selected from glioblastoma multiforme, bladder carcinoma, and esophageal carcinoma. In some embodiments, the cancer is triple-negative breast carcinoma. In some embodiments, the cancer may be a primary tumor. In some embodiments, the cancer may be a metastatic tumor at a second site derived from any of the above types of cancer. In some embodiments, a bispecific anti-MerTK:anti-PDL1 antibody of the present disclosure is useful for treating cancer in s subject in need thereof, wherein the cancer expresses MerTK.

In some embodiments, a bispecific anti-MerTK:anti-PDL1 antibody of the present disclosure may be administered in conjunction with one or more therapeutic agents that act as a checkpoint inhibitor. In some embodiments, the method further includes administering to the individual at least one antibody that specifically binds to an inhibitory immune checkpoint molecule, and/or another standard or investigational anti-cancer therapy. In some embodiments, the inhibitory checkpoint molecule is selected from PD1, PDL1, and PD-L2, In some embodiments, the at least one antibody that specifically binds to an inhibitory checkpoint molecule is administered in combination with the anti-MerTK antibody of the present disclosure

In some embodiments, the at least one antibody that specifically binds to an inhibitory checkpoint molecule is selected from an anti-PDL1 antibody, an anti-PD-L2 antibody, and an anti-PD-1 antibody

In some embodiments, a subject or individual is a mammal. Mammals include, without limitation, domesticated animals (e.g., cows, sheep, cats, dogs, and horses), primates (e.g., humans and non-human primates such as monkeys), rabbits, and rodents (e.g., mice and rats). In some embodiments, the subject or individual is a human.

### IX. Articles of Manufacture

Provided herein are articles of manufacture (e.g., kit) comprising a bispecific anti-MerTK:anti-PDL1 antibody described herein. Article of manufacture may include one or more containers comprising an antibody described herein. Containers may be any suitable packaging including, but is not limited to, vials, bottles, jars, flexible packaging (e.g., sealed Mylar or plastic bags), and the like. The containers may be unit doses, bulk packages (e.g., multi-dose packages) or sub-unit doses.

In some embodiments, the kits may further include a second agent. In some embodiments, the second agent is a pharmaceutically-acceptable buffer or diluting agent including. In some embodiments, the second agent is a pharmaceutically active agent.

In some embodiments of any of the articles of manufacture, the article of manufactures further include instructions for use in accordance with the methods of this disclosure. The instructions generally include information as to dosage, dosing schedule, and route of administration for the intended treatment. In some embodiments, these instructions comprise a description of administration of the isolated antibody of the present disclosure (e.g., a bispecific anti-MerTK:anti-PDL1 antibody described herein) to treat an individual having a disease, disorder, or injury, such as for example cancer, according to any methods of this disclosure. In some embodiments, the instructions include instructions for use of the bispecific anti-MerTK:anti-PDL1 antibody and the second agent (e.g., second pharmaceutically active agent).

The present disclosure will be more fully understood by reference to the following Examples. They should not, however, be construed as limiting the scope of the present disclosure. All citations throughout the disclosure are hereby expressly incorporated by reference.

### EXAMPLES

### Example 1: Production of His-Conjugated and Murine Fc-Conjugated MerTK Polypeptides

Human, cyno, and murine MerTK polypeptides containing polyHis or TEVS/Thrombin/murine IgG2a-Fc tagged fusion proteins for use in the generation and characterization of anti-MerTK antibodies of the present disclosure were generated as follows. Nucleic acid encoding the extracellular domain (ECD) of human MerTK (SEQ ID NO:2), cyno MerTK (SEQ ID NO:3), and murine MerTK (SEQ ID NO:4) were each cloned into a mammalian expression vector containing nucleic acid encoding a heterologous signal peptide as well as containing either a PolyHis Fc tag or TEVS/Thrombin/murine IgG2a Fc tag.

The amino acid sequences of human MerTK, human MerTK extracellular domain, cyno MerTK extracellular domain, and murine MerTK extracellular domain are set forth below.

Human MerTK amino acid sequence
(SEQ ID NO: 1):
mgpaplplllglflpalwrraiteareeakpyplfpgpfpgslqt
dhtpllslphasgyqpalmfsptqpgrphtgnvaipqvtsvesk
plpplafkhtvghiilsehkgvkfncsisvpniyqdttiswwkdg
kellgahhaitqfypddevtaiiasfsitsvqrsdngsyickmki
nneeivsdpiyievqglphftkqpesmnvtrntafnltcqavgpp
epvnifwvqnssrvneqpekspsvltvpgltemavfsceahndkg
ltvskgvqinikaipspptevsirnstahsiliswvpgfdgyspf
rncsiqvkeadplsngsvmifntsalphlyqikqlqalanysigv
scmneigwsavspwilasttegapsvaplnvtvflnessdnvdir
wmkpptkqqdgelvgyrishvwqsagiskelleevgqngsraris
vqvhnatctvriaavtrggvgpfsdpvkifipahgwvdyapsstp
apgnadpvliifgcfcgfiliglilyislairkrvqetkfgnaft
eedselvvnyiakksfcrraieltlhslgvseelqnkledvvidr
nllilgkilgegefgsvmegnlkqedgtslkvavktmkldnssqr
eieeflseaacmkdfshpnvirllgvciemssqgipkpmvilpfm
kygdlhtyllysrletgpkhiplqtllkfmvdialgmeylsnrnf
lhrdlaarncmlrddmtvcvadfglskkiysgdyyrqgriakmpv
kwiaiesladrvytsksdvwafgvtmweiatrgmtpypgvqnhem
ydyllhghrlkqpedcldelyeimyscwrtdpldrptfsvlrlql
eklleslpdvrnqadviyvntqllesseglaqgstlap1dlnidp
dsiiasctpraaisvvtaevhdskphegryilnggseewedltsa
psaavtaeknsvlpgerlvrngvswshssmlplgsslpdellfad
dssegsevlm
Human MerTK ECD amino acid sequence
(SEQ ID NO: 2):
MGPAPLPLLLGLFLPALWRRAITEAREEAKPYPLFPGPFPGSLQT
DHTPLLSLPHASGYQPALMFSPTQPGRPHTGNVAIPQVTSVESK
PLPPLAFKHTVGHIILSEHKGVKFNCSISVPNIYQDTTISWWKDG
KELLGAHHAITQFYPDDEVTAIIASFSITSVQRSDNGSYICKMKI
NNEEIVSDPIYIEVQGLPHFTKQPESMNVTRNTAFNLTCQAVGPP
EPVNIFWVQNSSRVNEQPEKSPSVLTVPGLTEMAVFSCEAHNDKG
LTVSKGVQINIKAIPSPPTEVSIRNSTAHSILISWVPGFDGYSPF
RNCSIQVKEADPLSNGSVMIFNTSALPHLYQIKQLQALANYSIGV
SCMNEIGWSAVSPWILASTTEGAPSVAPLNVTVFLNESSDNVDIR
WMKPPTKQQDGELVGYRISHVWQSAGISKELLEEVGQNGSRARIS
VQVHNATCTVRIAAVTRGGVGPFSDPVKIFIPAHGWVDYAPSSTP
APGNADPVLII
Cyno MerTK ECD amino acid sequence
(SEQ ID NO: 3):
MGLAPLPLPLLLGLFLPALWSRAITEAREEAKPYPLFPGPLPGSL
QTDHTSLLSLPHTSGYQPALMFSPTQPGRPYTGNVAIPRVTSAG
SKLLPPLAFKHTVGHIILSEHKDVKFNCSISVPNIYQDTTISWWK
DGKELLGAHHAITQFYPDDEVTAIIASFSITSVQRSDNGSYICKM
KINNEEIVSDPIYIEVQGLPHFTKQPESMNVTRNTAFNLTCQAVG
PPEPVNIFWVQNSSRVNEQPEKSPSVLTVPGLTEMAVFSCEAHND
KGLTVSKGVQINIKAIPSPPTEVSIHNSTAHSILISWVPGFDGYS
PFRNCSVQVKEVDPLSNGSVMIFNTSASPHMYQIKQLQALANYSI
GVSCMNEIGWSAVSPWILASTTEGAPSVAPLNVTVFLNESRDNVD
IRWMKPLTKRQAGELVGYRISHVWQSAGISKELLEEVGQNNSRAQ
ISVQVHNATCTVRIAAVTKGGVGPFSDPVKIFIPAHGWVDHAPSS
TPAPGNADPVLII
Murine MerTK ECD amino acid sequence
(SEQ ID NO: 4):
MVLAPLLLGLLLLPALWSGGTAEKWEETELDQLFSGPLPGRLPVN
HRPFSAPHSSRDQLPPPQTGRSHPAHTAAPQVTSTASKLLPPVA
FNHTIGHIVLSEHKNVKFNCSINIPNTYQETAGISWWKDGKELLG
AHHSITQFYPDEEGVSIIALFSIASVQRSDNGSYFCKMKVNNREI
VSDPIYVEVQGLPYFIKQPESVNVTRNTAFNLTCQAVGPPEPVNI
FWVQNSSRVNEKPERSPSVLTVPGLTETAVFSCEAHNDKGLTVSK
GVHINIKVIPSPPTEVHILNSTAHSILVSWVPGFDGYSPLQNCS
IQVKEADRLSNGSVMVENTSASPHLYEIQQLQALANYSIAVSCRN
EIGWSAVSPWILASTTEGAPSVAPLNITVFLNESNNILDIRWTKP
PIKRQDGELVGYRISHVWESAGTYKELSEEVSQNGSWAQIPVQIH
NATCTVRIAAITKGGIGPFSEPVNIIIPEHSKVDYAPSSTPAPGN
TDSM

The human, cyno, and murine MerTK nucleic acid fusion constructs were transiently transfected into HEK293 cells. The recombinant fusion polypeptides were purified from the supernatants of the cells using Mabselect resin (GE Healthcare, Cat #17519902) following the manufacturer's instructions. Additionally, commercially available DDDDK-tagged human MerTK fusion polypeptide (Sino Biological, Wayne, PA, Cat #10298-HCCH) or human IgG1 Fc-tagged murine MerTK fusion proteins (R&D systems, Minneapolis, MA, Cat #591-MR-100) were also used for anti-MerTK antibody characterization as described below.

### Example 2: Generation of Human and Murine MerTK Overexpressing CHO Cell Lines

Human MerTK and murine MerTK overexpressing CHO cell lines were prepared as follows. Human MerTK open reading frame (ORF) clone Lentivirus particle (Cat #RC215289L4V) and mouse MerTK ORF clone Lentivirus particle (Cat #MR225392L4V) (Origene, Rockville, MD) (both mGFP-tagged) were used for preparing human MerTK overexpressing CHO-K1 and murine MerTK overexpressing CHO-K1 stable cell line generation, respectively.

CHO cells were cultured in F12-K media (ATCC, Cat #ATCC 30-2004) containing 10% FBS (Gibco) until >80% confluent. The cells were then dissociated with Trypsin buffer (0.25% EDTA/Trypsin, Gibco, Cat #25200056) and plated at 70-80% confluency in 6-well plates 24 hours prior to transduction with either the human or murine MerTK lentivirus construct. The following day, cells were incubated with the lentiviral particle at 4° C. for 2 hours and then the plates were incubated at 37° C. in 5% CO₂. Two days later, puromycin (Invivogen, San Diego, CA, Cat #ant-pr-1) was added for selection; selected puromycin-resistant cells were frozen in Cell Recovery Freezing Medium (Gibco, Cat #12648010) for subsequent use.

For FACS analysis of these cell lines, human MerTK overexpressing CHO cells (CHO-huMerTK OE cells) and mouse MerTK overexpressing CHO cells (CHO-muMerTK OE cells) generated as described above were plated at 1-2×10⁵ cells per well in 96-well U-bottom plates and incubated with a commercially available mouse anti-human MerTK monoclonal antibody (BioLegend, Clone: 590H11G1E3, Cat #367608, San Diego, CA) or a commercially available rat anti-mouse MerTK monoclonal antibody (ThermoFisher, Clone: DS5MMER, Cat #12-5751-82) for 30 minutes on ice. Cells were rinsed twice with ice-cold FACS buffer (2% FBS+PBS) and then incubated with APC-conjugated goat anti-mouse antibody (Jackson ImmunoResearch, West Grove, PA, Cat #115-606-071) or goat-anti-rat antibody (Jackson ImmunoResearch, Cat #112-606-071) for 30 min on ice. Following the secondary antibody incubation, the cells were washed with ice-cold FACS buffer and then resuspended in a final volume of 50-200 μl of FACS buffer containing 0.25 μl/well propidium iodide (BD, Cat #556463). Analysis was performed using a FACS CantoII system (BD Biosciences).

The resulting human MerTK and murine MerTK overexpressing (OE) CHO cell lines were used for subsequent studies to characterize anti-MerTK antibodies as described below.

### Example 3: Generation of Anti-MerTK Hybridoma Antibodies

The following experiments were performed in order to generate anti-MerTK hybridomas. BALB/c mice (Charles River Laboratories, Wilmington, MA) or MerTK knock-out (KO) mice (Jackson Laboratories, Bar Harbor, ME) were immunized twice a week by subcutaneous or intraperitoneal injections of purified extracellular domain polypeptides of human, cyno, and mouse MerTK (obtained as described above in Example 1) with or without adjuvant. A total of 8 injections were performed over 4 weeks. Three days following the final injection, spleens and lymph nodes were harvested from the mice for hybridoma cell line generation.

Lymphocytes from the spleens and lymph nodes of the immunized mice were isolated and then fused with P3X63Ag8.653 (CRL-1580, American Type Culture Collection, Rockville, MD) or SP2/mIL-6 (CRL-2016, American Type Culture Collection, Rockville, MD) mouse myeloma cells via electrofusion (Hybrimmune, BTX, Holliston, MA) and incubated at 37° C., 5% CO₂, overnight in Clonacell-HY Medium C (STEMCELL Technologies, Vancouver, BC, Canada, Cat #03803). The following day, the fused cells were centrifuged and resuspended in 10 ml of ClonaCell-HY Medium C with anti-mouse IgG Fc-FITC (Jackson ImmunoResearch, West Grove, PA) and then gently mixed with 90 ml of methylcellulose-based ClonaCell-HY Medium D (STEMCELL Technologies, Cat #03804) containing HAT components. The cells were plated into Nunc OmniTrays (Thermo Fisher Scientific, Rochester, NY) and allowed to grow at 37° C., 5% CO₂ for seven days. Fluorescent colonies were then selected and transferred into 96-well plates containing Clonacell-HY Medium E (STEMCELL Technologies, Cat #03805) using a Clonepix 2 (Molecular Devices, Sunnyvale, CA). After six days of culture, tissue culture supernatants from the hybridomas were screened by FACS analysis for specificity to bind human MerTK or mouse MerTK as described below.

### Example 4: Screening of Anti-MerTK Antibody Hybridoma Supernatants by FACS

Hybridoma culture supernatants obtained as described above were screened for their ability to bind MerTK on various cell types, including CHO cells stably overexpressing human MerTK (CHO-huMerTK OE cells) or stably overexpressing mouse MerTK (CHO-muMerTK OE cells) (generated as described above), and CHO parental cells; U937 cells (ATCC CRL-1593.2), SK-MEL-5 cells (ATCC HTB-70) (which endogenously express human MerTK), J774A. 1 cells (ATCC TIB-67) (which endogenously express mouse MerTK), and A375 cells (ATCC CRL-1619). THP-1 cells (ATCC TIB-202), which have no or minimal expression of MerTK, served as non-MerTK expressing negative control cells in these experiments.

For screening of the hybridoma cell culture supernatants, a multiplexed FACS experimental design was utilized to determine binding of anti-MerTK antibodies to these multiple cell lines. Briefly, cells were stained with various concentrations and combinations of CellTrace cell proliferation dyes CFSE and Violet (ThermoFisher, Cat #C34554 and Cat #34557, respectively) to create uniquely barcoded cell populations. 70,000 cells of each barcoded cell type were aliquoted into 96-well U-bottom plates and incubated with 50 μl of hybridoma cell culture supernatant or 5 μg/ml of commercially available purified mouse anti-human MerTK monoclonal antibody (BioLegend, Cat #367602; serving as a positive anti-MerTK antibody) on ice for 30 minutes. After this primary anti-MerTK hybridoma supernatant incubation with the various MerTK expressing cell types, the supernatants were removed via centrifugation, the cells were washed twice with 175 μl of ice-cold FACS buffer (PBS+1% FBS+2 mM EDTA), and the cells were then further incubated on ice for 20 minutes with anti-mouse IgG Fc-allophycocyanin (APC) (Jackson Labs, Cat #115-136-071) (diluted 1:1000). Following this secondary antibody incubation, the cells were again washed twice with ice-cold FACS buffer and resuspended in a final volume of 30 μl of FACS buffer containing 0.25 μl/well propidium iodide (BD Biosciences, Cat #556463). Binding intensity on cells was analyzed using the FACS Canto system (BD Biosciences), with sorting gates drawn to exclude dead (i.e., propidium iodide-positive) cells. The ratio of APC Mean Fluorescence Intensity (MFI) on each barcoded cell population was determined for each anti-MerTK hybridoma supernatant tested.

From this specific hybridoma supernatant screen, anti-MerTK hybridoma clones were identified that displayed greater than 2-fold difference in binding (as determined by MFI) to cells stably overexpressing or endogenously expressing human or mouse MerTK compared to the binding observed on parental or negative control cell types. Anti-MerTK antibodies identified using this screen were further characterized as described below.

### Example 5: Screening of Anti-MerTK Antibody Hybridoma Supernatants by Recombinant MerTK Protein Binding Assay

Hybridoma culture supernatants obtained as described above were screened for their ability to bind polyHis-tagged human, cyno, and mouse MerTK (prepared as described above in Example 1) as compared to binding to an irrelevant His-tagged control protein. Briefly, 96-well polystyrene plates were coated with lug/ml of human, cyno, or mouse poly-His-tagged MerTK polypeptide in coating buffer (0.05M carbonate buffer, pH 9.6, Sigma, Cat #C3041) overnight at 4° C. Coated plates were then blocked with ELISA diluent (PBS+0.5% BSA+0.05% Tween20) for one hour and washed three times with 300 μl of PBST (PBS+0.05% Tween20, Thermo 28352). The hybridoma cell culture supernatants or two commercially available purified mouse anti-human MerTK monoclonal antibodies (BioLegend Cat #367602; R&D Cat #MAB8912) were added (50 μl/well) to each well. After 30 mins incubation (room temperature, with shaking), the plates were washed three times with 300 μl of PBST. Anti-mouse IgG Fc-HRP (Jackson Immunoresearch, Cat #115-035-071) secondary antibody was diluted 1:5000 in ELISA diluent, added to each well at 50 μl/well, and incubated for 30 minutes at room temperature with shaking. After a final set of washes (3×300 μl in PBST), 50 μl/well of TMB substrate (BioFx, Cat #TMBW-1000-01) was added to the wells. The reaction was then quenched after 5-10 mins with 50 μl/well of stop solution (BioFx, Cat #BSTP-1000-01). The quenched reaction wells were detected for absorbance at 650 nm with a BioTek Synergy Microplate Reader using GEN5 2.04 software. From this hybridoma supernatant screen, anti-MerTK hybridoma clones were identified that displayed greater than 10-fold difference in binding to recombinant MerTK over background. Anti-MerTK antibodies identified using this screen were further characterized as described below.

### Example 6: Molecular Cloning of Anti-MerTK Antibodies

Anti-MerTK antibodies from the hybridomas described above were subcloned as follows. 5×10⁵ hybridoma cells were harvested and washed with PBS and then the cell pellets were flash frozen in dry ice and stored at −20° C. Total RNA was extracted by using RNeasy Mini Kit (QIAGEN, Cat #74104) following the manufacturer's protocol. cDNA was generated using Clontech's SMARTer RACE 5′/3′ Kit (Takara Bio USA, Cat #634859) following the manufacturer's protocol. Variable heavy and light immunoglobulin regions were cloned separately by touchdown PCR using the 5′ UPM primer provided in the RACE kit and reverse primers recognizing the heavy chain and light chain constant regions. The resulting PCR products were purified and ligated into a pCR2.1-TOPO cloning vector (TOPO TA cloning Kit, Invitrogen Cat #450641) and transformed into Escherichia coli (E. coli) cells. Transformed colonies were isolated and the variable heavy chain (VH) and variable light chain (VL) nucleic acids were sequenced for each corresponding hybridoma cell line. Following the sequence determinations, variable heavy chain regions and variable light chain regions were amplified by PCR using primers containing endonuclease restriction sites and then subcloned into pLEV-123 (LakePharma, San Carlos, CA) mammalian expression vector encoding human IgG1-Fc-LALAPS (human IgG1 Fc comprising amino acid substitutions L234A, L235A, and P331S by EU numbering) and IgG Kappa. Anti-MerTK antibodies of the present disclosure obtained as described above include anti-MerTK antibody MTK-16, MTK-33, and MTK-15.

### Example 7: Humanization and Affinity Maturation of Mouse Anti-MERTK Antibodies

Humanized variants of certain parental mouse anti-MerTK antibodies of the present disclosure were generated as follows.

One method of humanizing non-human antibodies is to transplant the CDRs from a non-human (e.g., murine) antibody onto a human antibody acceptor framework. Such CDR transplantation may result in attenuation or complete loss of affinity of the humanized antibody to its target due to perturbation in its framework. As a result, certain amino acid residues in the human framework may need to be replaced by amino acid residues from the corresponding positions of the murine antibody framework (referred to as back mutations) in order to restore attenuated or lost affinity as a result of humanization. Therefore, the amino acid residues to be replaced in the context of the selected human antibody germline acceptor framework must be determined so that the humanized antibody substantially retains functions and paratopes. In addition, retained or improved thermal stability and solubility are desired for good manufacturability and downstream development.

Briefly, VH and VL amino acid sequences of the mouse anti-MerTK monoclonal antibodies to be humanized were compared to human VL, VH, LJ, and HJ functional germline amino acid sequences taken from IMGT (http://www.imgt.org/). Pseudo-genes and open reading frames were excluded from these analyses. Per one mouse monoclonal antibody (query), one or two of the most similar VH and one of the most similar VL germline amino acid sequences were selected and combined with the most similar VJ and HJ genes, producing one or two humanized amino acid sequences. The CDRs to be transplanted onto the human framework were defined according to the AbM definition.

The query and the humanized amino acid sequences were used to create Fv homology models using BioMOE module or the Antibody Modeler module of MOE (Molecular Operating Environment, Chemical Computing Group, Montreal, Canada). AMBER10:EHT force field analysis was used for energy minimization through the entire antibody homology modeling process. Based on the Fv homology models obtained, molecular descriptors such as interaction energy between VL and VH, coordinate-based isoelectric point (3D pI), hydrophobic patch, and charged surface area were calculated, analyzed, and sorted by scoring metrics provided by MOE. These molecular descriptors were utilized to prioritize the humanized monoclonal antibodies for downstream experimental procedures, including protein expression, purification, binding affinity studies, and functional assays.

The BioMOE module of MOE provides a tool, Mutation Site Properties, to visualize and classify potential residues for back-mutation. In this context, back-mutation is defined as amino acid substitution which is reverted to the original query amino acid sequence replacing the humanized amino acid sequence. Using this tool, the original query (reference) was compared individually to the selected humanized variants for both the primary amino acid sequence and the 3D structure of the 3D Fv homology model.

Changes between the reference (i.e., parental) antibody and the humanized variant were classified based on amino acid type difference, interaction potential with CDR residues, impact potential for VL/VH pairing, and potential change in hydrophobic and charged surface area in and near the CDRs.

Mutations near the CDRs or the VL/VH interface having a significant charge difference or containing strong H-bond interactions were individually evaluated and the significantly disrupting mutations were reverted to the original query residues.

Affinity maturation of humanized anti-MerTK antibodies MTK-33 and MTK-16 were performed. Briefly, certain amino acid residues in the heavy chain or light chain were selectively mutagenized and mutants that improved binding were selected through additional rounds of screening. This process simultaneously improved specificity, species cross-reactivity, and developability profiles. Characterization of the affinity-matured anti-MerTK antibodies described herein included SPR affinity measurements on Carterra LSA and efferocytosis blocking assays on human macrophage. After multiple rounds of affinity maturation, anti-MerTK antibodies with desired affinity were obtained.

Amino acid sequences of the variable heavy chains (VH) and variable light (VL) chains of anti-MerTK antibodies of the present disclosure are provided below in Table 1. In Table 1, parental mouse anti-MerTK antibodies include MTK-15, MTK-16, and MTK-33; humanized and affinity matured variants of MTK-16 and MTK-33 are MTK-16.2 and MTK-33.11, respectively. In Table 1, the hypervariable regions (HVR) in each of the antibody chains are underlined.

TABLE 1

| | | SEQ | | SEQ |
|---|---|---|---|---|
| | | ID | | ID |
| Antibody | Heavy Chain Variable | NO: | Light Chain Variable | NO: |
| MTK-15 | QIQLVQSGPELKKPGETVKIS | 5 | QIVLSQSPAILSASPGEKVTMT | 6 |
| | CKASGYTFTNYGVHWVKQAPG | | CRASSSVSYMHWYQQKSGSSPK | |
| | KGLKWMGWINTYTGEPTYADD | | PWIYATSNLASGVPARFSGSGS | |
| | FKGRFAFSLETSASTAYLQIN | | GTSYSLTISRVEAEDAATYYCQ | |
| | NLKNEDMATYFCARGNRYAYM | | QWSSNPRTFGGGTKLEIK | |
| | DYWGQGTSVTVSS | | | |
| MTK-16 | LIQLVQSGPELKKPGETVKIS | 7 | DIVMTQSPKFMSTSVGDRVSIT | 8 |
| | CKASGYTFTNHGMNWVKQDPG | | CKASQNVRTAVAWYKKKPGQSP | |
| | KGLKWMGWINTYTGEPTYADD | | KALINLASNRHTGVPDRFTGSG | |
| | FKGRFVFSMETSASAAFLQIN | | SGTDFTLTISNVQSEDLADYFC | |
| | NLKNEDTATYFCARKGVTAAR | | LQHWNYPLTFGGGTKLEIK | |
| | YFDYWGQGTTLTVSS | | | |
| MTK-16.2 | QVQLVQSGSELKKPGASVKVS | 9 | DIQMTQSPSSLSASVGDRVTIT | 10 |
| | CKASGYTFTNHGMNWVRQAPG | | CRASQNVRTAVAWYQQKPGKAP | |
| | QGLEWMGWINTYTGEPTYADD | | KRLIYLASNRHTGVPSRFSGSG | |
| | FKGRFVFSLDTSVSTAYLQIS | | SGTEFTLTISNLQPEDFATYYC | |
| | SLKAEDTAVYYCARKGVTAAR | | LQHWNYPLTFGGGTKLEIK | |
| | YFDYWGQGTLVTVSS | | | |
| MTK-33 | QVQLQQPGPELVRPGTSVKLS | 11 | SIVMTQTPKFLLVSAGDRVIIT | 12 |
| | CKASGYTFTSYWMHWIKQRPG | | CKASQSVSNTVAWYQQKPGQSP | |
| | QGLEWIGVIDPSDNYINYNQK | | KLLIYYASNRYTGVPDRFTGSG | |
| | FKGKATLTVDTSSSTAYLQLS | | YGTDFTFTISTVQAEDLAVYFC | |
| | SLTSEDSAVYYCAREAGTRGY | | QQDYRSPFTFGSGTQLEMK | |
| | FDYWGQGTTLTVSS | | | |
| MTK-33.11 | QVQLVQSGAEVKKPGASVKVS | 13 | DIQMTQSPSSLSASVGDRVTIT | 14 |
| | CKASGYTFTSYWMHWVRQAPG | | CQASRSVRNTVAWYQQKPGKAP | |
| | QGLEWIGVIDPSDNYINYNQK | | KLLIYYASNRYTGVPSRFSGSG | |
| | FQGRVTMTRDTSTSTVYMELS | | SGTDFTFTISSLQPEDIATYYC | |
| | SLRSEDTAVYYCAREAWTRGY | | QQDYRSPFTFGQGTKLEIK | |
| | FDYWGQGTLVTVSS | | | |
| PDL1 | EVQLVESGGGLVQPGGSLRLS | 52 | DIQMTQSPSSLSASVGDRVTIT | 53 |
| | CAASGFTFSDSWIHWVRQAPG | | CRASQDVSTAVAWYQQKPGKAP | |
| | KGLEWVAWISPYGGSTYYADS | | KLLIYSASFLYSGVPSRFSGSG | |
| | VKGRFTISADTSKNTAYLQMN | | SGTDFTLTISSLQPEDFATYYC | |
| | SLRAEDTAVYYCARRHWPGGF | | QQYLYHPATFGQGTKVEIK | |
| | DYWGQGTLVTVSS | | | |

### Example 8: Anti-MerTK Antibodies of the Present Disclosure and Anti-PDL1 Antibodies Comprising Various Fc Regions for Use in Bispecific Antibody Constructs

Heavy chain antibody variable sequences were introduced into various IgG Fc regions: wild-type human IgG1; human IgG1 comprising LALAPS modifications (L234A, L235A, P331S; EU numbering); human IgG1 comprising NSLF (N325S, L328F; EU numbering); and wild-type human IgG4. Light chain antibody variable sequences were introduced into the human IgG light chain constant region. The resulting anti-MerTk antibody sequences are provided below in Table 2:

TABLE 2

| | | SEQ ID |
|---|---|---|
| Antibody | Amino Acid Sequence | NO |
| MTK-15 | QIQLVQSGPELKKPGETVKISCKASGYTFTNYGVHWVKQAPGKGL | 15 |
| LALAPS | KWMGWINTYTGEPTYADDFKGRFAFSLETSASTAYLQINNLKNED | |
| heavy chain | MATYFCARGNRYAYMDYWGQGTSVTVSSASTKGPSVFPLAPSSKS | |
| | TSGGTAALGCLVKDYFPEPVTVSWNSGALTSGVHTFPAVLQSSGL | |
| | YSLSSVVTVPSSSLGTQTYICNVNHKPSNTKVDKKVEPKSCDKTH | |
| | TCPPCPAPEAAGGPSVFLFPPKPKDTLMISRTPEVTCVVVDVSHE | |
| | DPEVKFNWYVDGVEVHNAKTKPREEQYNSTYRVVSVLTVLHQDWL | |
| | NGKEYKCKVSNKALPASIEKTISKAKGQPREPQVYTLPPSRDELT | |
| | KNQVSLTCLVKGFYPSDIAVEWESNGQPENNYKTTPPVLDSDGSF | |
| | FLYSKLTVDKSRWQQGNVFSCSVMHEALHNHYTQKSLSLSPGK | |
| MTK-15 | QIVLSQSPAILSASPGEKVTMTCRASSSVSYMHWYQQKSGSSPKP | 16 |
| light chain | WIYATSNLASGVPARFSGSGSGTSYSLTISRVEAEDAATYYCQQW | |
| | SSNPRTFGGGTKLEIKRTVAAPSVFIFPPSDEQLKSGTASVVCLL | |
| | NNFYPREAKVQWKVDNALQSGNSQESVTEQDSKDSTYSLSSTLTL | |
| | SKADYEKHKVYACEVTHQGLSSPVTKSFNRGEC | |
| MTK-16.2 | QVQLVQSGSELKKPGASVKVSCKASGYTFTNHGMNWVRQAPGQGL | 17 |
| wild-type | EWMGWINTYTGEPTYADDFKGRFVFSLDTSVSTAYLQISSLKAED | |
| IgG1 | TAVYYCARKGVTAARYFDYWGQGTLVTVSSASTKGPSVFPLAPSS | |
| heavy chain | KSTSGGTAALGCLVKDYFPEPVTVSWNSGALTSGVHTFPAVLQSS | |
| | GLYSLSSVVTVPSSSLGTQTYICNVNHKPSNTKVDKKVEPKSCDK | |
| | THTCPPCPAPELLGGPSVFLFPPKPKDTLMISRTPEVTCVVVDVS | |
| | HEDPEVKFNWYVDGVEVHNAKTKPREEQYNSTYRVVSVLTVLHQD | |
| | WLNGKEYKCKVSNKALPAPIEKTISKAKGQPREPQVYTLPPSRDE | |
| | LTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGK | |
| MTK-16.2 | QVQLVQSGSELKKPGASVKVSCKASGYTFTNHGMNWVRQAPGQGL | 18 |
| LALAPS | EWMGWINTYTGEPTYADDFKGRFVFSLDTSVSTAYLQISSLKAED | |
| heavy chain | TAVYYCARKGVTAARYFDYWGQGTLVTVSSASTKGPSVFPLAPSS | |
| | KSTSGGTAALGCLVKDYFPEPVTVSWNSGALTSGVHTFPAVLQSS | |
| | GLYSLSSVVTVPSSSLGTQTYICNVNHKPSNTKVDKKVEPKSCDK | |
| | THTCPPCPAPEAAGGPSVFLFPPKPKDTLMISRTPEVTCVVVDVS | |
| | HEDPEVKFNWYVDGVEVHNAKTKPREEQYNSTYRVVSVLTVLHQD | |
| | WLNGKEYKCKVSNKALPASIEKTISKAKGQPREPQVYTLPPSRDE | |
| | LTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGK | |
| MTK-16.2 | QVQLVQSGSELKKPGASVKVSCKASGYTFTNHGMNWVRQAPGQGL | 19 |
| NSLF | EWMGWINTYTGEPTYADDFKGRFVFSLDTSVSTAYLQISSLKAED | |
| heavy chain | TAVYYCARKGVTAARYFDYWGQGTLVTVSSASTKGPSVFPLAPSS | |
| | KSTSGGTAALGCLVKDYFPEPVTVSWNSGALTSGVHTFPAVLQSS | |
| | GLYSLSSVVTVPSSSLGTQTYICNVNHKPSNTKVDKKVEPKSCDK | |
| | THTCPPCPAPELLGGPSVFLFPPKPKDTLMISRTPEVTCVVVDVS | |
| | HEDPEVKFNWYVDGVEVHNAKTKPREEQYNSTYRVVSVLTVLHQD | |
| | WLNGKEYKCKVSSKAFPAPIEKTISKAKGQPREPQVYTLPPSRDE | |
| | LTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGK | |
| MTK-16.2 | QVQLVQSGSELKKPGASVKVSCKASGYTFTNHGMNWVRQAPGQGL | 20 |
| IgG4 | EWMGWINTYTGEPTYADDFKGRFVFSLDTSVSTAYLQISSLKAED | |
| heavy chain | TAVYYCARKGVTAARYFDYWGQGTLVTVSSASTKGPSVFPLAPCS | |
| | RSTSESTAALGCLVKDYFPEPVTVSWNSGALTSGVHTFPAVLQSS | |
| | GLYSLSSVVTVPSSSLGTKTYTCNVDHKPSNTKVDKRVESKYGPP | |
| | CPSCPAPEFLGGPSVFLFPPKPKDTLMISRTPEVTCVVVDVSQED | |
| | PEVQFNWYVDGVEVHNAKTKPREEQFNSTYRVVSVLTVLHQDWLN | |
| | GKEYKCKVSNKGLPSSIEKTISKAKGQPREPQVYTLPPSQEEMTK | |
| | NQVSLTCLVKGFYPSDIAVEWESNGQPENNYKTTPPVLDSDGSFF | |
| | LYSRLTVDKSRWQEGNVFSCSVMHEALHNHYTQKSLSLSLGK | |
| MTK-16.2 | DIQMTQSPSSLSASVGDRVTITCRASQNVRTAVAWYQQKPGKAPK | 21 |
| light chain | RLIYLASNRHTGVPSRFSGSGSGTEFTLTISNLQPEDFATYYCLQ | |
| | HWNYPLTFGGGTKLEIKRTVAAPSVFIFPPSDEQLKSGTASVVCL | |
| | LNNFYPREAKVQWKVDNALQSGNSQESVTEQDSKDSTYSLSSTLT | |
| | LSKADYEKHKVYACEVTHQGLSSPVTKSFNRGEC | |
| MTK-33.11 | QVQLVQSGAEVKKPGASVKVSCKASGYTFTSYWMHWVRQAPGQGL | 22 |
| wild-type | EWIGVIDPSDNYINYNQKFQGRVTMTRDTSTSTVYMELSSLRSED | |
| IgG1 | TAVYYCAREAWTRGYFDYWGQGTLVTVSSASTKGPSVFPLAPSSK | |
| heavy chain | STSGGTAALGCLVKDYFPEPVTVSWNSGALTSGVHTFPAVLQSSG | |
| | LYSLSSVVTVPSSSLGTQTYICNVNHKPSNTKVDKKVEPKSCDKT | |
| | HTCPPCPAPELLGGPSVFLFPPKPKDTLMISRTPEVTCVVVDVSH | |
| | EDPEVKFNWYVDGVEVHNAKTKPREEQYNSTYRVVSVLTVLHQDW | |
| | LNGKEYKCKVSNKALPAPIEKTISKAKGQPREPQVYTLPPSRDEL | |
| | TKNQVSLTCLVKGFYPSDIAVEWESNGQPENNYKTTPPVLDSDGS | |
| | FFLYSKLTVDKSRWQQGNVFSCSVMHEALHNHYTQKSLSLSPGK | |
| MTK-33.11 | QVQLVQSGAEVKKPGASVKVSCKASGYTFTSYWMHWVRQAPGQGL | 23 |
| LALAPS | EWIGVIDPSDNYINYNQKFQGRVTMTRDTSTSTVYMELSSLRSED | |
| heavy chain | TAVYYCAREAWTRGYFDYWGQGTLVTVSSASTKGPSVFPLAPSSK | |
| | STSGGTAALGCLVKDYFPEPVTVSWNSGALTSGVHTFPAVLQSSG | |
| | LYSLSSVVTVPSSSLGTQTYICNVNHKPSNTKVDKKVEPKSCDKT | |
| | HTCPPCPAPEAAGGPSVFLFPPKPKDTLMISRTPEVTCVVVDVSH | |
| | EDPEVKFNWYVDGVEVHNAKTKPREEQYNSTYRVVSVLTVLHQDW | |
| | LNGKEYKCKVSNKALPASIEKTISKAKGQPREPQVYTLPPSRDEL | |
| | TKNQVSLTCLVKGFYPSDIAVEWESNGQPENNYKTTPPVLDSDGS | |
| | FFLYSKLTVDKSRWQQGNVFSCSVMHEALHNHYTQKSLSLSPGK | |
| MTK-33.11 | QVQLVQSGAEVKKPGASVKVSCKASGYTFTSYWMHWVRQAPGQGL | 24 |
| NSLF | EWIGVIDPSDNYINYNQKFQGRVTMTRDTSTSTVYMELSSLRSED | |
| heavy chain | TAVYYCAREAWTRGYFDYWGQGTLVTVSSASTKGPSVFPLAPSSK | |
| | STSGGTAALGCLVKDYFPEPVTVSWNSGALTSGVHTFPAVLQSSG | |
| | LYSLSSVVTVPSSSLGTQTYICNVNHKPSNTKVDKKVEPKSCDKT | |
| | HTCPPCPAPELLGGPSVFLFPPKPKDTLMISRTPEVTCVVVDVSH | |
| | EDPEVKFNWYVDGVEVHNAKTKPREEQYNSTYRVVSVLTVLHQDW | |
| | LNGKEYKCKVSSKAFPAPIEKTISKAKGQPREPQVYTLPPSRDEL | |
| | TKNQVSLTCLVKGFYPSDIAVEWESNGQPENNYKTTPPVLDSDGS | |
| | FFLYSKLTVDKSRWQQGNVFSCSVMHEALHNHYTQKSLSLSPGK | |
| MTK-33.11 | QVQLVQSGAEVKKPGASVKVSCKASGYTFTSYWMHWVRQAPGQGL | 25 |
| IgG4 | EWIGVIDPSDNYINYNQKFQGRVTMTRDTSTSTVYMELSSLRSED | |
| heavy chain | TAVYYCAREAWTRGYFDYWGQGTLVTVSSASTKGPSVFPLAPCSR | |
| | STSESTAALGCLVKDYFPEPVTVSWNSGALTSGVHTFPAVLQSSG | |
| | LYSLSSVVTVPSSSLGTKTYTCNVDHKPSNTKVDKRVESKYGPPC | |
| | PSCPAPEFLGGPSVFLFPPKPKDTLMISRTPEVTCVVVDVSQEDP | |
| | EVQFNWYVDGVEVHNAKTKPREEQFNSTYRVVSVLTVLHQDWLNG | |
| | KEYKCKVSNKGLPSSIEKTISKAKGQPREPQVYTLPPSQEEMTKN | |
| | QVSLTCLVKGFYPSDIAVEWESNGQPENNYKTTPPVLDSDGSFFL | |
| | YSRLTVDKSRWQEGNVFSCSVMHEALHNHYTQKSLSLSLGK | |
| MTK-33.11 | DIQMTQSPSSLSASVGDRVTITCQASRSVRNTVAWYQQKPGKAPK | 26 |
| light chain | LLIYYASNRYTGVPSRFSGSGSGTDFTFTISSLQPEDIATYYCQQ | |
| | DYRSPFTFGQGTKLEIKRTVAAPSVFIFPPSDEQLKSGTASVVCL | |
| | LNNFYPREAKVQWKVDNALQSGNSQESVTEQDSKDSTYSLSSTLT | |
| | LSKADYEKHKVYACEVTHQGLSSPVTKSFNRGEC | |
| Anti-PDL1 | EVQLVESGGGLVQPGGSLRLSCAASGFTFSDSWIHWVRQAPGKGL | 27 |
| Wildtype | EWVAWISPYGGSTYYADSVKGRFTISADTSKNTAYLQMNSLRAED | |
| IgG1 | TAVYYCARRHWPGGFDYWGQGTLVTVSSASTKGPSVFPLAPSSKS | |
| Heavy chain | TSGGTAALGCLVKDYFPEPVTVSWNSGALTSGVHTFPAVLQSSGL | |
| | YSLSSVVTVPSSSLGTQTYICNVNHKPSNTKVDKKVEPKSCDKTH | |
| | TCPPCPAPELLGGPSVFLFPPKPKDTLMISRTPEVTCVVVDVSHE | |
| | DPEVKFNWYVDGVEVHNAKTKPREEQYNSTYRVVSVLTVLHQDWL | |
| | NGKEYKCKVSNKALPAPIEKTISKAKGQPREPQVYTLPPSRDELT | |
| | KNQVSLTCLVKGFYPSDIAVEWESNGQPENNYKTTPPVLDSDGSF | |
| | FLYSKLTVDKSRWQQGNVFSCSVMHEALHNHYTQKSLSLSPGK | |
| Anti-PDL1 | EVQLVESGGGLVQPGGSLRLSCAASGFTFSDSWIHWVRQAPGKGL | 28 |
| LALAPS | EWVAWISPYGGSTYYADSVKGRFTISADTSKNTAYLQMNSLRAED | |
| heavy chain | TAVYYCARRHWPGGFDYWGQGTLVTVSSASTKGPSVFPLAPSSKS | |
| | TSGGTAALGCLVKDYFPEPVTVSWNSGALTSGVHTFPAVLQSSGL | |
| | YSLSSVVTVPSSSLGTQTYICNVNHKPSNTKVDKKVEPKSCDKTH | |
| | TCPPCPAPEAAGGPSVFLFPPKPKDTLMISRTPEVTCVVVDVSHE | |
| | DPEVKFNWYVDGVEVHNAKTKPREEQYNSTYRVVSVLTVLHQDWL | |
| | NGKEYKCKVSNKALPASIEKTISKAKGQPREPQVYTLPPSRDELT | |
| | KNQVSLTCLVKGFYPSDIAVEWESNGQPENNYKTTPPVLDSDGSF | |
| | FLYSKLTVDKSRWQQGNVFSCSVMHEALHNHYTQKSLSLSPGK | |
| Anti-PDL1 | EVQLVESGGGLVQPGGSLRLSCAASGFTFSDSWIHWVRQAPGKGL | 29 |
| NSLF | EWVAWISPYGGSTYYADSVKGRFTISADTSKNTAYLQMNSLRAED | |
| Heavy chain | TAVYYCARRHWPGGFDYWGQGTLVTVSSASTKGPSVFPLAPSSKS | |
| | TSGGTAALGCLVKDYFPEPVTVSWNSGALTSGVHTFPAVLQSSGL | |
| | YSLSSVVTVPSSSLGTQTYICNVNHKPSNTKVDKKVEPKSCDKTH | |
| | TCPPCPAPELLGGPSVFLFPPKPKDTLMISRTPEVTCVVVDVSHE | |
| | DPEVKFNWYVDGVEVHNAKTKPREEQYNSTYRVVSVLTVLHQDWL | |
| | NGKEYKCKVSSKAFPAPIEKTISKAKGQPREPQVYTLPPSRDELT | |
| | KNQVSLTCLVKGFYPSDIAVEWESNGQPENNYKTTPPVLDSDGSF | |
| | FLYSKLTVDKSRWQQGNVFSCSVMHEALHNHYTQKSLSLSPGK | |
| Anti-PDL1 | EVQLVESGGGLVQPGGSLRLSCAASGFTFSDSWIHWVRQAPGKGL | 30 |
| IgG4 | EWVAWISPYGGSTYYADSVKGRFTISADTSKNTAYLQMNSLRAED | |
| Heavy chain | TAVYYCARRHWPGGFDYWGQGTLVTVSSASTKGPSVFPLAPCSRS | |
| | TSESTAALGCLVKDYFPEPVTVSWNSGALTSGVHTFPAVLQSSGL | |
| | YSLSSVVTVPSSSLGTKTYTCNVDHKPSNTKVDKRVESKYGPPCP | |
| | SCPAPEFLGGPSVFLFPPKPKDTLMISRTPEVTCVVVDVSQEDPE | |
| | VQFNWYVDGVEVHNAKTKPREEQFNSTYRVVSVLTVLHQDWLNGK | |
| | EYKCKVSNKGLPSSIEKTISKAKGQPREPQVYTLPPSQEEMTKNQ | |
| | VSLTCLVKGFYPSDIAVEWESNGQPENNYKTTPPVLDSDGSFFLY | |
| | SRLTVDKSRWQEGNVFSCSVMHEALHNHYTQKSLSLSLGK | |
| Anti-PDL1 | DIQMTQSPSSLSASVGDRVTITCRASQDVSTAVAWYQQKPGKAPK | 31 |
| light chain | LLIYSASFLYSGVPSRFSGSGSGTDFTLTISSLQPEDFATYYCQQ | |
| | YLYHPATFGQGTKVEIKRTVAAPSVFIFPPSDEQLKSGTASVVCL | |
| | LNNFYPREAKVQWKVDNALQSGNSQESVTEQDSKDSTYSLSSTLT | |
| | LSKADYEKHKVYACEVTHQGLSSPVTKSFNRGEC | |

Table 2 above also includes the amino acid sequence for an anti-PDL1 antibody comprising the heavy chain variable region and light chain variable region from anti-PDL1 antibody atezolizumab comprising human IgG1 Fc wildtype, human IgG1 Fc LALAPS, human IgG1 Fc NSLF, human IgG4, and human IgG light chain, respectively.

### Example 9: Production of Anti-MerTK Antibodies

Anti-MerTK hybridoma clones were cultured in serum free hybridoma media and the anti-MerTK antibodies in the supernatants purified on Hamilton STAR platform (Hamilton Company, Reno, NV) using Protein A tips (Phynexus Inc, San Jose, CA). Anti-MerTK antibodies were also produced via direct cloning of the variable gene regions obtained from the hybridomas into a recombinant expression plasmid for production of chimeric antibodies containing a human Fc domain (human IgG1 containing LALAPS amino acid substitutions described above). Using the Tuna293™ Process (LakePharma, San Carlos, CA), HEK293 cell were seeded into shake flasks and expanded using serum-free chemically defined media. The expression plasmids were transiently transfected into the cells and the culture supernatants were harvested 7 days later. After clarification by centrifugation and filtration, the anti-MerTK antibodies in the supernatants were purified via Protein A chromatography.

### Example 10: Bispecific Anti-MerTK:Anti-PDL1 Antibody Sequences

Table 3 shows amino acid sequences for various anti-MerTK:anti-PDL1 bispecific antibodies of the present disclosure, comprising “knob” amino acid modifications in the Fc regions of the heavy chain anti-MerTK antibody sequences, and comprising “hole” amino acid modifications in the Fc region of the heavy chain anti-PDL1 amino acid sequence, associated with Fc region heterodimerization. In Table 3, IgG1 and IgG4 Fc region modifications for “knob” configurations comprise the amino acid substitution T366W (EU numbering), and “hole” configurations comprise the amino acid substitutions T366S, L368A, and Y407V. Certain IgG4 configurations also include Fc region hinge modifications comprising the amino acid substitution S228P (EU numbering) to prevent Fab arm exchange (See Silva et al, (2015), J Biol Chem, 290:5462-5469). Specifically, wildtype hinge region of hulgG4 comprises the amino acid sequence ESKYGPPCPSCP (SEQ ID NO:54) whereas the S228P amino acid substitution of IgG4 hinge region comprises the amino acid sequence ESKYGPPCPPCP (SEQ ID NO:55).

TABLE 3

| | | |
|---|---|---|
| | | SEQ ID |
| Antibody | Amino Acid Sequence | NO |

MTK-16.2

| | | |
|---|---|---|
| MTK-16.2 | QVQLVQSGSELKKPGASVKVSCKASGYTFTNHGMNWVRQAPGQ | 32 |
| IgG1 knob | GLEWMGWINTYTGEPTYADDFKGRFVFSLDTSVSTAYLQISSL | |
| Heavy chain | KAEDTAVYYCARKGVTAARYFDYWGQGTLVTVSSASTKGPSVF | |
| | PLAPSSKSTSGGTAALGCLVKDYFPEPVTVSWNSGALTSGVHT | |
| | FPAVLQSSGLYSLSSVVTVPSSSLGTQTYICNVNHKPSNTKVD | |
| | KKVEPKSCDKTHTCPPCPAPELLGGPSVFLFPPKPKDTLMISR | |
| | TPEVTCVVVDVSHEDPEVKFNWYVDGVEVHNAKTKPREEQYNS | |
| | TYRVVSVLTVLHQDWLNGKEYKCKVSNKALPASELFPIEKTIS | |
| | KAKGQPREPQVYTLPPSRDELTKNQVSLWCLVKGFYPSDIAVE | |
| | WESNGQPENNYKTTPPVLDSDGSFFLYSKLTVDKSRWQQGNVF | |
| | SCSVMHEALHNHYTQKSLSLSPGK | |
| MTK-16.2 | QVQLVQSGSELKKPGASVKVSCKASGYTFTNHGMNWVRQAPGQ | 33 |
| LALAPS knob | GLEWMGWINTYTGEPTYADDFKGRFVFSLDTSVSTAYLQISSL | |
| Heavy chain | KAEDTAVYYCARKGVTAARYFDYWGQGTLVTVSSASTKGPSVF | |
| | PLAPSSKSTSGGTAALGCLVKDYFPEPVTVSWNSGALTSGVHT | |
| | FPAVLQSSGLYSLSSVVTVPSSSLGTQTYICNVNHKPSNTKVD | |
| | KKVEPKSCDKTHTCPPCPAPEAAGGPSVFLFPPKPKDTLMISR | |
| | TPEVTCVVVDVSHEDPEVKFNWYVDGVEVHNAKTKPREEQYNS | |
| | TYRVVSVLTVLHQDWLNGKEYKCKVSNKALPASIEKTISKAKG | |
| | QPREPQVYTLPPSRDELTKNQVSLWCLVKGFYPSDIAVEWESN | |
| | GQPENNYKTTPPVLDSDGSFFLYSKLTVDKSRWQQGNVFSCSV | |
| | MHEALHNHYTQKSLSLSPGK | |
| MTK-16.2 | QVQLVQSGSELKKPGASVKVSCKASGYTFTNHGMNWVRQAPGQ | 34 |
| NSLF knob | GLEWMGWINTYTGEPTYADDFKGRFVFSLDTSVSTAYLQISSL | |
| Heavy chain | KAEDTAVYYCARKGVTAARYFDYWGQGTLVTVSSASTKGPSVF | |
| | PLAPSSKSTSGGTAALGCLVKDYFPEPVTVSWNSGALTSGVHT | |
| | FPAVLQSSGLYSLSSVVTVPSSSLGTQTYICNVNHKPSNTKVD | |
| | KKVEPKSCDKTHTCPPCPAPELLGGPSVFLFPPKPKDTLMISR | |
| | TPEVTCVVVDVSHEDPEVKFNWYVDGVEVHNAKTKPREEQYNS | |
| | TYRVVSVLTVLHQDWLNGKEYKCKVSSKAFPAPIEKTISKAKG | |
| | QPREPQVYTLPPSRDELTKNQVSLTCLVKGFYPSDIAVEWESN | |
| | GQPENNYKTTPPVLDSDGSFFLYSKLTVDKSRWQQGNVFSCSV | |
| | MHEALHNHYTQKSLSLSPGK | |
| MTK-16.2 | QVQLVQSGSELKKPGASVKVSCKASGYTFTNHGMNWVRQAPGQ | 35 |
| IgG4 knob | GLEWMGWINTYTGEPTYADDFKGRFVFSLDTSVSTAYLQISSL | |
| | KAEDTAVYYCARKGVTAARYFDYWGQGTLVTVSSASTKGPSVF | |
| | PLAPCSRSTSESTAALGCLVKDYFPEPVTVSWNSGALTSGVHT | |
| | FPAVLQSSGLYSLSSVVTVPSSSLGTKTYTCNVDHKPSNTKVD | |
| | KRVESKYGPPCPSCPAPEFLGGPSVFLFPPKPKDTLMISRTPE | |
| | VTCVVVDVSQEDPEVQFNWYVDGVEVHNAKTKPREEQFNSTYR | |
| | VVSVLTVLHQDWLNGKEYKCKVSNKGLPSSIEKTISKAKGQPR | |
| | EPQVYTLPPSQEEMTKNQVSLWCLVKGFYPSDIAVEWESNGQP | |
| | ENNYKTTPPVLDSDGSFFLYSRLTVDKSRWQEGNVFSCSVMHE | |
| | ALHNHYTQKSLSLSLGK | |
| MTK-16.2 | QVQLVQSGSELKKPGASVKVSCKASGYTFTNHGMNWVRQAPGQG | 36 |
| IgG4 knob | LEWMGWINTYTGEPTYADDFKGRFVFSLDTSVSTAYLQISSLKA | |
| S228P | EDTAVYYCARKGVTAARYFDYWGQGTLVTVSSASTKGPSVFPLA | |
| Heavy chain | PCSRSTSESTAALGCLVKDYFPEPVTVSWNSGALTSGVHTFPAV | |
| | LQSSGLYSLSSVVTVPSSSLGTKTYTCNVDHKPSNTKVDKRVES | |
| | KYGPPCPPCPAPEFLGGPSVFLFPPKPKDTLMISRTPEVTCVVV | |
| | DVSQEDPEVQFNWYVDGVEVHNAKTKPREEQFNSTYRVVSVLTV | |
| | LHQDWLNGKEYKCKVSNKGLPSSIEKTISKAKGQPREPQVYTLP | |
| | PSQEEMTKNQVSLWCLVKGFYPSDIAVEWESNGQPENNYKTTPP | |
| | VLDSDGSFFLYSRLTVDKSRWQEGNVFSCSVMHEALHNHYTQKS | |
| | LSLSLGK | |

MTK-33.11

| | | |
|---|---|---|
| MTK-33.11 | QVQLVQSGAEVKKPGASVKVSCKASSYSFTSYWMHWVRQAPGQ | 37 |
| IgG1 knob | GLEWIGVIDPSDNYINYNQKFRGRVTMTRDTSTSTVYMELSSL | |
| Heavy chain | RSEDTAVYYCAREAGTRGYFDYWGQGTLVTVSSASTKGPSVFP | |
| | LAPSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCDKTHTCPPCPAPELLGGPSVFLFPPKPKDTLMISRT | |
| | PEVTCVVVDVSHEDPEVKFNWYVDGVEVHNAKTKPREEQYNST | |
| | YRVVSVLTVLHQDWLNGKEYKCKVSNKALPAPIEKTISKAKGQ | |
| | PREPQVYTLPPSRDELTKNQVSLWCLVKGFYPSDIAVEWESNG | |
| | QPENNYKTTPPVLDSDGSFFLYSKLTVDKSRWQQGNVFSCSVM | |
| | HEALHNHYTQKSLSLSPGK | |
| MTK-33.11 | QVQLVQSGAEVKKPGASVKVSCKASSYSFTSYWMHWVRQAPGQ | 38 |
| LALAPS knob | GLEWIGVIDPSDNYINYNQKFRGRVTMTRDTSTSTVYMELSSL | |
| Heavy chain | RSEDTAVYYCAREAGTRGYFDYWGQGTLVTVSSASTKGPSVFP | |
| | LAPSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCDKTHTCPPCPAPEAAGGPSVFLFPPKPKDTLMISRT | |
| | PEVTCVVVDVSHEDPEVKFNWYVDGVEVHNAKTKPREEQYNST | |
| | YRVVSVLTVLHQDWLNGKEYKCKVSNKALPASIEKTISKAKGQ | |
| | PREPQVYTLPPSRDELTKNQVSLWCLVKGFYPSDIAVEWESNG | |
| | QPENNYKTTPPVLDSDGSFFLYSKLTVDKSRWQQGNVFSCSVM | |
| | HEALHNHYTQKSLSLSPGK | |
| MTK-33.11 | QVQLVQSGAEVKKPGASVKVSCKASSYSFTSYWMHWVRQAPGQ | 39 |
| NSLF knob | GLEWIGVIDPSDNYINYNQKFRGRVTMTRDTSTSTVYMELSSL | |
| Heavy chain | RSEDTAVYYCAREAGTRGYFDYWGQGTLVTVSSASTKGPSVFP | |
| | LAPSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCDKTHTCPPCPAPELLGGPSVFLFPPKPKDTLMISRT | |
| | PEVTCVVVDVSHEDPEVKFNWYVDGVEVHNAKTKPREEQYNST | |
| | YRVVSVLTVLHQDWLNGKEYKCKVSSKAFPAPIEKTISKAKGQ | |
| | PREPQVYTLPPSRDELTKNQVSLWCLVKGFYPSDIAVEWESNG | |
| | QPENNYKTTPPVLDSDGSFFLYSKLTVDKSRWQQGNVFSCSVM | |
| | HEALHNHYTQKSLSLSPGK | |
| MTK-33.11 | QVQLVQSGAEVKKPGASVKVSCKASSYSFTSYWMHWVRQAPGQ | 40 |
| IgG4 knob | GLEWIGVIDPSDNYINYNQKFRGRVTMTRDTSTSTVYMELSSL | |
| Heavy chain | RSEDTAVYYCAREAGTRGYFDYWGQGTLVTVSSASTKGPSVFP | |
| | LAPCSRSTSESTAALGCLVKDYFPEPVTVSWNSGALTSGVHTF | |
| | PAVLQSSGLYSLSSVVTVPSSSLGTKTYTCNVDHKPSNTKVDK | |
| | RVESKYGPPCPSCPAPEFLGGPSVFLFPPKPKDTLMISRTPEV | |
| | TCVVVDVSQEDPEVQFNWYVDGVEVHNAKTKPREEQFNSTYRV | |
| | VSVLTVLHQDWLNGKEYKCKVSNKGLPSSIEKTISKAKGQPRE | |
| | PQVYTLPPSQEEMTKNQVSLWCLVKGFYPSDIAVEWESNGQPE | |
| | NNYKTTPPVLDSDGSFFLYSRLTVDKSRWQEGNVFSCSVMHEA | |
| | LHNHYTQKSLSLSLGK | |
| MTK-33.11 | QVQLVQSGAEVKKPGASVKVSCKASSYSFTSYWMHWVRQAPGQ | 41 |
| IgG4 knob | GLEWIGVIDPSDNYINYNQKFRGRVTMTRDTSTSTVYMELSSL | |
| S228P | RSEDTAVYYCAREAGTRGYFDYWGQGTLVTVSSASTKGPSVFP | |
| Heavy chain | LAPCSRSTSESTAALGCLVKDYFPEPVTVSWNSGALTSGVHTF | |
| | PAVLQSSGLYSLSSVVTVPSSSLGTKTYTCNVDHKPSNTKVDK | |
| | RVESKYGPPCPPCPAPEFLGGPSVFLFPPKPKDTLMISRTPEV | |
| | TCVVVDVSQEDPEVQFNWYVDGVEVHNAKTKPREEQFNSTYRV | |
| | VSVLTVLHQDWLNGKEYKCKVSNKGLPSSIEKTISKAKGQPRE | |
| | PQVYTLPPSQEEMTKNQVSLWCLVKGFYPSDIAVEWESNGQPE | |
| | NNYKTTPPVLDSDGSFFLYSRLTVDKSRWQEGNVFSCSVMHEA | |
| | LHNHYTQKSLSLSLGK | |

Anti-PDL1

| | | |
|---|---|---|
| Anti-PDL1 | EVQLVESGGGLVQPGGSLRLSCAASGFTFSDSWIHWVRQAPGK | 42 |
| IgG hole | GLEWVAWISPYGGSTYYADSVKGRFTISADTSKNTAYLQMNSL | |
| heavy chain | RAEDTAVYYCARRHWPGGFDYWGQGTLVTVSSASTKGPSVFPL | |
| | APSSKSTSGGTAALGCLVKDYFPEPVTVSWNSGALTSGVHTFP | |
| | AVLQSSGLYSLSSVVTVPSSSLGTQTYICNVNHKPSNTKVDKK | |
| | VEPKSCDKTHTCPPCPAPELLGGPSVFLFPPKPKDTLMISRTP | |
| | EVTCVVVDVSHEDPEVKFNWYVDGVEVHNAKTKPREEQYNSTY | |
| | RVVSVLTVLHQDWLNGKEYKCKVSNKALPAPIEKTISKAKGQP | |
| | REPQVYTLPPSRDELTKNQVSLSCAVKGFYPSDIAVEWESNGQ | |
| | PENNYKTTPPVLDSDGSFFLVSKLTVDKSRWQQGNVFSCSVMH | |
| | EALHNHYTQKSLSLSPGK | |
| Anti-PDL1 | EVQLVESGGGLVQPGGSLRLSCAASGFTFSDSWIHWVRQAPGK | 43 |
| LALAPS hole | GLEWVAWISPYGGSTYYADSVKGRFTISADTSKNTAYLQMNSL | |
| Heavy chain | RAEDTAVYYCARRHWPGGFDYWGQGTLVTVSSASTKGPSVFPL | |
| | APSSKSTSGGTAALGCLVKDYFPEPVTVSWNSGALTSGVHTFP | |
| | AVLQSSGLYSLSSVVTVPSSSLGTQTYICNVNHKPSNTKVDKK | |
| | VEPKSCDKTHTCPPCPAPEAAGGPSVFLFPPKPKDTLMISRTP | |
| | EVTCVVVDVSHEDPEVKFNWYVDGVEVHNAKTKPREEQYNSTY | |
| | RVVSVLTVLHQDWLNGKEYKCKVSNKALPASIEKTISKAKGQP | |
| | REPQVYTLPPSRDELTKNQVSLSCAVKGFYPSDIAVEWESNGQ | |
| | PENNYKTTPPVLDSDGSFFLVSKLTVDKSRWQQGNVFSCSVMH | |
| | EALHNHYTQKSLSLSPGK | |
| Anti-PDL1 | EVQLVESGGGLVQPGGSLRLSCAASGFTFSDSWIHWVRQAPGK | 44 |
| NSLF hole | GLEWVAWISPYGGSTYYADSVKGRFTISADTSKNTAYLQMNSL | |
| Heavy chain | RAEDTAVYYCARRHWPGGFDYWGQGTLVTVSSASTKGPSVFPL | |
| | APSSKSTSGGTAALGCLVKDYFPEPVTVSWNSGALTSGVHTFP | |
| | AVLQSSGLYSLSSVVTVPSSSLGTQTYICNVNHKPSNTKVDKK | |
| | VEPKSCDKTHTCPPCPAPELLGGPSVFLFPPKPKDTLMISRTP | |
| | EVTCVVVDVSHEDPEVKFNWYVDGVEVHNAKTKPREEQYNSTY | |
| | RVVSVLTVLHQDWLNGKEYKCKVSSKAFPAPIEKTISKAKGQP | |
| | REPQVYTLPPSRDELTKNQVSLSCAVKGFYPSDIAVEWESNGQ | |
| | PENNYKTTPPVLDSDGSFFLVSKLTVDKSRWQQGNVFSCSVMH | |
| | EALHNHYTQKSLSLSPGK | |
| Anti-PDL1 | EVQLVESGGGLVQPGGSLRLSCAASGFTFSDSWIHWVRQAPGK | 45 |
| IgG4 hole | GLEWVAWISPYGGSTYYADSVKGRFTISADTSKNTAYLQMNSL | |
| Heavy chain | RAEDTAVYYCARRHWPGGFDYWGQGTLVTVSSASTKGPSVFPL | |
| | APCSRSTSESTAALGCLVKDYFPEPVTVSWNSGALTSGVHTFP | |
| | AVLQSSGLYSLSSVVTVPSSSLGTKTYTCNVDHKPSNTKVDKR | |
| | VESKYGPPCPSCPAPEFLGGPSVFLFPPKPKDTLMISRTPEVT | |
| | CVVVDVSQEDPEVQFNWYVDGVEVHNAKTKPREEQFNSTYRVV | |
| | SVLTVLHQDWLNGKEYKCKVSNKGLPSSIEKTISKAKGQPREP | |
| | QVYTLPPSQEEMTKNQVSLSCAVKGFYPSDIAVEWESNGQPEN | |
| | NYKTTPPVLDSDGSFFLVSRLTVDKSRWQEGNVFSCSVMHEAL | |
| | HNHYTQKSLSLSLGK | |
| Anti-PDL1 | EVQLVESGGGLVQPGGSLRLSCAASGFTFSDSWIHWVRQAPGK | 46 |
| IgG4 hole | GLEWVAWISPYGGSTYYADSVKGRFTISADTSKNTAYLQMNSL | |
| S228P | RAEDTAVYYCARRHWPGGFDYWGQGTLVTVSSASTKGPSVFPL | |
| Heavy chain | APCSRSTSESTAALGCLVKDYFPEPVTVSWNSGALTSGVHTFP | |
| | AVLQSSGLYSLSSVVTVPSSSLGTKTYTCNVDHKPSNTKVDKR | |
| | VESKYGPPCPPCPAPEFLGGPSVFLFPPKPKDTLMISRTPEVT | |
| | CVVVDVSQEDPEVQFNWYVDGVEVHNAKTKPREEQFNSTYRVV | |
| | SVLTVLHQDWLNGKEYKCKVSNKGLPSSIEKTISKAKGQPREP | |
| | QVYTLPPSQEEMTKNQVSLSCAVKGFYPSDIAVEWESNGQPEN | |
| | NYKTTPPVLDSDGSFFLVSRLTVDKSRWQEGNVFSCSVMHEAL | |
| | HNHYTQKSLSLSLGK | |

TABLE 4

| Antibody Fc | | SEQ ID |
|---|---|---|
| hole | Amino Acid Sequence | NO |
| IgG1 wildtype | DKTHTCPPCPAPELLGGPSVFLFPPKPKDTLMISRTPEVTCVV | 47 |
| Fc hole | VDVSHEDPEVKFNWYVDGVEVHNAKTKPREEQYNSTYRVVSVL | |
| | TVLHQDWLNGKEYKCKVSNKALPAPIEKTISKAKGQPREPQVY | |
| | TLPPSRDELTKNQVSLSCAVKGFYPSDIAVEWESNGQPENNYK | |
| | TTPPVLDSDGSFFLVSKLTVDKSRWQQGNVFSCSVMHEALHNH | |
| | YTQKSLSLSPGK | |
| IgG1 LALAPS | DKTHTCPPCPAPEAAGGPSVFLFPPKPKDTLMISRTPEVTCVV | 48 |
| Fc hole | VDVSHEDPEVKFNWYVDGVEVHNAKTKPREEQYNSTYRVVSVL | |
| | TVLHQDWLNGKEYKCKVSNKALPASIEKTISKAKGOPREPQVY | |
| | TLPPSRDELTKNQVSLSCAVKGFYPSDIAVEWESNGQPENNYK | |
| | TTPPVLDSDGSFFLVSKLTVDKSRWQQGNVFSCSVMHEALHNH | |
| | YTQKSLSLSPGK | |
| IgG1 NSLF | DKTHTCPPCPAPELLGGPSVFLFPPKPKDTLMISRTPEVTCVV | 49 |
| Fc hole | VDVSHEDPEVKFNWYVDGVEVHNAKTKPREEQYNSTYRVVSVL | |
| | TVLHQDWLNGKEYKCKVSSKAFPAPIEKTISKAKGOPREPQVY | |
| | TLPPSRDELTKNQVSLSCAVKGFYPSDIAVEWESNGQPENNYK | |
| | TTPPVLDSDGSFFLVSKLTVDKSRWQQGNVFSCSVMHEALHNH | |
| | YTQKSLSLSPGK | |
| IgG4 wildtype | ESKYGPPCPSCPAPEFLGGPSVFLFPPKPKDTLMISRTPEVTC | 50 |
| Fc hole | VVVDVSQEDPEVQFNWYVDGVEVHNAKTKPREEQFNSTYRVVS | |
| | VLTVLHQDWLNGKEYKCKVSNKGLPSSIEKTISKAKGQPREPQ | |
| | VYTLPPSQEEMTKNQVSLSCAVKGFYPSDIAVEWESNGOPENN | |
| | YKTTPPVLDSDGSFFLVSRLTVDKSRWQEGNVFSCSVMHEALH | |
| | NHYTQKSLSLSLGK | |
| IgG4 S228P | ESKYGPPCPPCPAPEFLGGPSVFLFPPKPKDTLMISRTPEVTCV | 51 |
| Fc hole | VVDVSQEDPEVQFNWYVDGVEVHNAKTKPREEQFNSTYRVVSVL | |
| | TVLHQDWLNGKEYKCKVSNKGLPSSIEKTISKAKGQPREPQVYT | |
| | LPPSQEEMTKNQVSLSCAVKGFYPSDIAVEWESNGOPENNYKTT | |
| | PPVLDSDGSFFLVSRLTVDKSRWQEGNVFSCSVMHEALHNHYTQ | |
| | KSLSLSLGK | |

### Example 11: Binding Activity of Bispecific Anti-MerTK:Anti-PDL1 Antibodies to CHO-Human MerTK OE Cells, CHO-Human PDL1 OE Cells, and M2c-Differentiated Human Macrophages

To determine whether bispecific anti-MerTK:anti-PDL1 antibodies of the present disclosure bind to different cell types, the following experiments were performed. Cells used in these studies included CHO cells overexpressing human MerTK (CHO-human MerTK OE cells), CHO cells overexpressing human PDL1 (CHO-human PDL1 OE cells), and M2c-differentiated human macrophages.

Human macrophages were differentiated from human monocytes for 7 days in the presence of human M-CSF, resulting in M2c-differentiated human macrophages. After 7 days, the differentiated human macrophages were harvested (by scraping), resuspended in PBS, and plated on 96-well plates for use. CHO cells overexpressing human MerTK (CHO-huMerTK OE), CHO cells overexpressing human PDL1 (CHO-huPDL1 OE), and M2c-differentiated human macrophages were stained with Dylight650-conjugated bispecific anti-MerTK:anti-PDL1 antibodies, or bivalent anti-MerTK antibodies for 30 min on ice. A range of antibody concentrations were used in these studies to obtain binding curves. All antibodies tested in these studies were IgG1 having Fc regions comprising LALAPS mutations. Cells were fixed and then acquired on a BD FACS Canto II cytometer. The mean fluorescence intensity (MFI) was calculated using FlowJo software and Kd value was calculated using Prism software. Kd values in these results were similar to EC50 values but are calculated by Prism software from data generated by FACS analysis. A lower Kd value reflects higher binding of antibody to the cells.

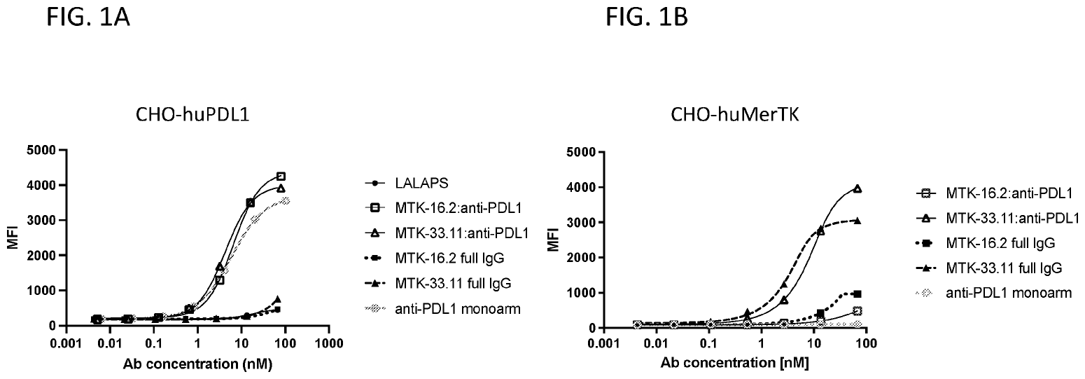

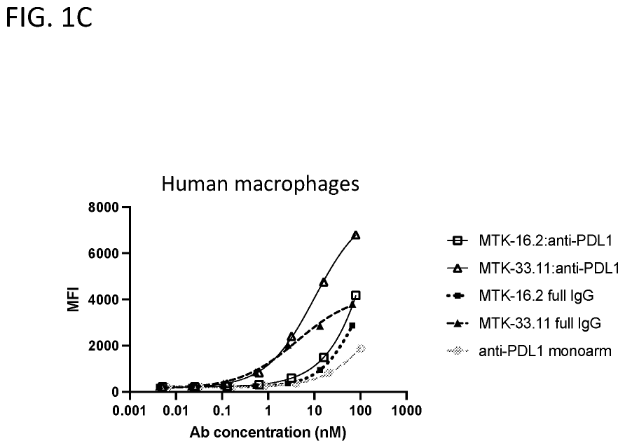

FIG. 1A shows the binding curves of bispecific anti-MerTK MTK-16.2:anti-PDL1 antibody, bispecific anti-MerTK MTK-33.11:anti-PDL1 antibody, bivalent anti-MerTK antibody MTK-16.2 (full IgG), bivalent anti-MerTK antibody MTK33.11 (full IgG), monovalent anti-PDL1 antibody, and IgG1-Fc LALAPS control antibody on CHO-huPDL1 OE cells.

FIG. 1B shows the binding curves of bispecific anti-MerTK MTK-16.2:anti-PDL1 antibody, bispecific anti-MerTK MTK-33.11:anti-PDL1 antibody, bivalent anti-MerTK antibody MTK-16.2, bivalent anti-MerTK antibody MTK33.11, and monovalent (monoarm) anti-PDL1 antibody on CHO-huMerTK OE cells.

As shown in FIG. 1A, bispecific anti-MerTK MTK-16.2:anti-PDL1 antibody and bispecific anti-MerTK MTK-33.11:anti-PDL1 antibody showed similar binding to CHO cells overexpressing human PDL1. Additionally, these two bispecific antibodies showed similar binding to CHO cells expressing human PDL1 as a monovalent (monoarm) configuration of anti-PDL1 antibody. These data indicated that bispecific anti-MerTK:anti-PDL1 antibodies of the present disclosure maintain their ability to bind human PDL1 expressed in cells.

As shown in FIG. 1B, bispecific anti-MerTK-MTK-33.11:anti-PDL1 antibody bound to CHO cells overexpressing human MerTK to a similar extent as that observed with bivalent (full IgG) anti-MerTK antibody MTK-33.11. Additionally, bispecific anti-MerTK-MTK-16.2:anti-PDL1 antibody bound to CHO cells overexpressing human MerTK to a similar extent as that observed with bivalent (full IgG) anti-MerTK antibody MTK-16.2. As expected, monovalent (monoarm) anti-PDL1 antibody did not show binding to CHO cells overexpressing human MerTK.

FIG. 1C shows binding of bispecific antibodies of the present disclosure on M2c-differentiated human macrophages.

Table 5 below summarizes the Kd values (nM) obtained from the FACS analyses. As shown in FIGS. 1A and 1B and in Table 5, both monovalent anti-MerTK antibody MTK-16.2 and bivalent anti-MerTK antibody MTK-16.2 showed binding to CHO-huMerTK OE cells with similar affinities in the approximate range of 40-43 nM. Monovalent anti-MerTK antibody MTK-16.2 and bivalent anti-MerTK antibody MTK-16.2 showed comparable binding to M2c-differentiated human macrophages, with affinities of approximately 78 nM and 52 nM, respectively, as measured by this assay. These results indicated that anti-MerTK antibody MTK-16.2 binds MerTK with similar affinity regardless of the antibody having one or two binding arms.

Bivalent anti-MerTK antibody MTK-33.11 displayed higher binding affinity to both CHO-huMerTK OE cells and M2c-differentiated human macrophages compared to that of monovalent anti-MerTK antibody MTK-33.11, displaying approximately 10-fold higher affinity to CHO-huMerTK OE cells and approximately 5-fold higher affinity to M2c-differentiated human macrophages compared to its monovalent form. As expected, control antibody monovalent anti-PDL1 did not show binding to CHO-huMerTK OE cells but did bind to M2c-differentiated human macrophages.

TABLE 5

| | Average | | |
|---|---|---|---|
| | FACS Kd value | | |
| | (nM) | | |
| | CHO- | CHO- | |
| | huPDL1 | huMerTK | M2c- |
| Antibody | OE | OE | huMacrophages |
| Anti-PDL 1 monovalent | 1.658 | No biding | 38.0 |
| | | detected | |
| MTK-16.2 monovalent | No binding | 40.45 | 77.78 |
| | detected | | |
| MTK-16.2:anti-PDL1 | 1.573 | 48.34 | 38.75 |
| bispecific | | | |
| MTK-16.2 bivalent | No binding | 42.49 | 51.87 |
| | detected | | |
| MTK-33.11 monovalent | No binding | 4.967 | 9.965 |
| | detected | | |
| MTK-33.11:anti-PDL1 | 1.134 | 3.247 | 5.457 |
| bispecific | | | |
| MTK-33.11 bivalent | No binding | 0.439 | 1.998 |
| | detected | | |

Monovalent anti-PDL1 antibody and bispecific antibodies bound to CHO-huPDL1 OE cells with a similar Kd value. Interestingly, bispecific anti-MerTK antibody MTK-16.2:anti-PDL1 showed stronger binding activity than monovalent anti-MerTK antibody MTK-16.2 or bivalent anti-MerTK antibody MTK-16.2 on M2c-differentiated human macrophages, suggesting that bispecific anti-MerTK antibody MTK-16.2:anti-PDL1 may synergistically bind to human macrophages via MerTK and PDL1. The results further suggested that bispecific anti-MerTK antibody MTK-33.11:anti-PDL1 is more dependent on bivalency for binding due to a relative strong affinity to MerTK, while binding of bispecific anti-MerTK antibody MTK-16.2:anti-PDL1 may be relying on PDL1 binding because of its weaker affinity to MerTK.

### Example 12: Reducing Efferocytosis with Bispecific Anti-MerTK:Anti-PDL1 Antibodies

The ability of bispecific anti-MerTK: anti-PDL1 antibodies of the present disclosure to reduce efferocytosis by phagocytic cells (e.g., human macrophages) was evaluated as follows. Human macrophages were differentiated from human monocytes for 7 days in the presence of human M-CSF to obtain M2c-differentiated human macrophages as described above. After 7 days, the M2c-differentiated human macrophages were harvested (by scraping), resuspended in PBS, and plated on 96-well plates. For efferocytosis IC50 determinations, cells were starved for 1 hour followed by the addition anti-MerTK antibody to each well for 30 min at 37° C.

Jurkat cells were treated with 1 μM staurosporin (SigmaAldrich) for 3 hours at 37° C. (to induce apoptosis) and labeled with pHrodo (ThermoFisher) for 30 min at room temperature. After washing with PBS, pHrodo labeled Jurkat cells were added into each well containing the human macrophages at 1:4 ratio (1 macrophage:4 Jurkat cells) for 1 hour. The plates were washed with PBS and then the cells were stained with APC-conjugated anti-human CD14 for 30 minutes on ice in the dark. Cells were fixed and then acquired on a BD FACS Canto II cytometer. Data were analyzed using FlowJo software.

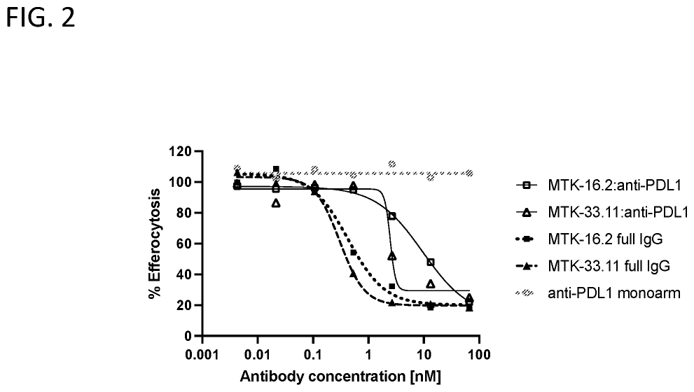

In these experiments, efferocytosis-positive macrophages were identified by setting pHrodo CD14 double positive cells as an analysis gate and then applying this exact gate to all the samples. Baseline efferocytosis levels were established using macrophages cultured with media alone and this was set to 100% efferocytosis activity. Relative efferocytosis levels were calculated as a percent of efferocytosis observed in cells treated with media alone compared to that observed in cells treated with anti-MerTK antibodies. Results of these studies are shown in FIG. 2 and in Table 6 below.

TABLE 6

| | Average |
|---|---|
| | Efferocytosis blocking |
| Antibody | IC50 (nM) |
| MTK-16.2 monovalent | 37.86 |
| MTK-16.2:anti-PDL1 bispecific | 15.68 |
| MTK-16.2 bivalent | 0.436 |
| MTK-33.11 monovalent | 4.447 |
| MTK-33.11:anti-PDL1 bispecific | 4.417 |
| MTK-33.11 bivalent | 0.341 |

As shown in Table 6, monovalent anti-MerTK antibodies of the present disclosure were able to block efferocytosis by M2c-differentiated human macrophages with an IC50 of approximately 4.4 nM and 37.9 nM for monovalent anti-MerTK antibodies MTK-33.11 and MTK-16.2, respectively. These data also showed that monovalent anti-MerTK antibodies MTK-16.2 and MTK-33.11 displayed relatively weaker efferocytosis reducing activity compared to their bivalent antibody counterparts; bivalent anti-MTK-33.11 antibody showed an IC50 value of 0.341 nM, whereas monovalent anti-MerTK antibody MTK-33.11 had an IC50 value of about 4.4 nM; further, bivalent anti-MTK-16.2 antibody had an IC50 value of 0.436 nM, whereas monovalent anti-MerTK antibody MTK-16.2 had an IC50 value of 37.86 nM. Monovalent anti-PDL1 antibody showed no inhibitory activity on efferocytosis (see FIG. 2).

These data showed that bispecific anti-MerTK:anti-PDL1 antibodies of the present disclosure were effective at reducing efferocytosis by phagocytic cells.

### Example 13: pAKT Activity of Bispecific Anti-MerTK:Anti-PDL1 Antibodies

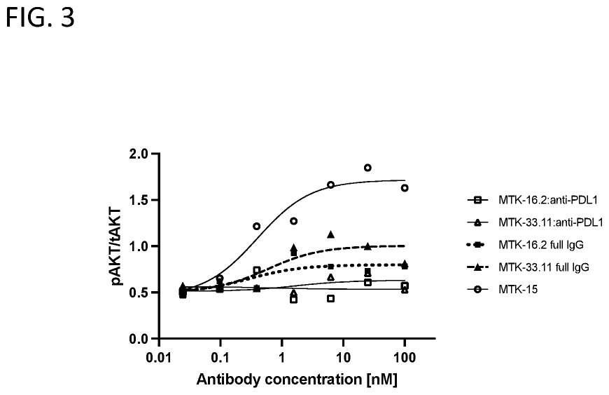

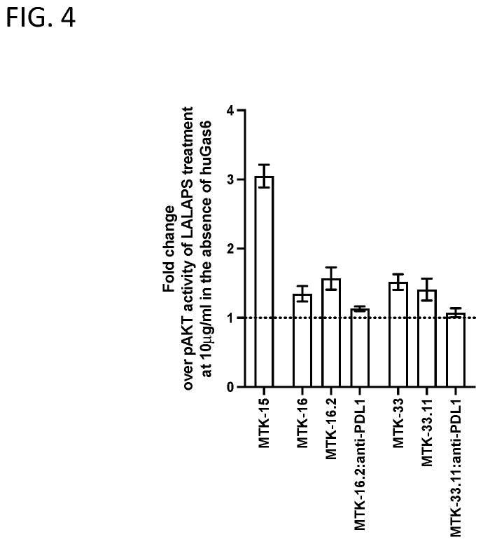

The ability of bispecific anti-MerTK:anti-PDL1 antibodies of the present disclosure to either increase or reduce pAKT signaling in the absence or presence of the MerTK ligand Gas6 was examined as follows. M2c-differentiated human macrophages (generated as described above) were treated with various anti-MerTK antibodies (10 μg/ml) for 30 min at 37° C., followed by the addition of 200 nM of recombinant human Gas6 or PBS. After an additional 30 min incubation, the cells were lysed and analyzed for pAKT levels in the cells compared to total AKT using homogenous time resolved fluorescence (HTRF) assays. The pAKT signal was normalized to total AKT signal to quantitate a final pAKT activity determination. Results of these studies are shown in FIG. 3 and Table 7. FIG. 4 shows fold-change in pAKT activity in the absence of huGas6.

TABLE 7

| | Average fold change to |
|---|---|
| | pAKT activity of |
| | LALAPS treatment |
| | at 10 μg/ml |
| Antibody | in the absence of huGas6 |
| MTK-16.2 monovalent | 1.220 |
| MTK-16.2:anti-PDL1 bispecific | 1.133 |
| MTK-16.2 bivalent | 1.568 |
| MTK-33.11 monovalent | 1.288 |
| MTK-33.11:anti-PDL1 bispecific | 1.074 |
| MTK-33.11 bivalent | 1.407 |

As shown in FIG. 3 and Table 7, monovalent anti-MerTK antibodies MTK-16.2 and MTK-33.11 showed a lower fold-change in pAKT/tAKT in the absence of the MerTK ligand Gas6 compared to their bivalent counterparts. This data indicated that monovalent anti-MerTK antibodies of the present disclosure are less effective at increasing pAKT levels in human macrophages in the absence of the MerTK ligand Gas6 (e.g., ligand-independent pAKT activity) compared to that observed with bivalent anti-MerTK antibodies of the present disclosure. Anti-MerTK antibody MTK-15 served as a positive control antibody that is effective at increasing pAKT activity. As it may be desirable to have less pAKT activity associated with anti-MerTK antibody administration, these data suggested that use of a bispecific anti-MerTK:anti-PDL1 antibody may provide a safer toxicity profile compared to using a combination of a bivalent anti-MerTK antibody and a bivalent anti-PDL1 antibody in a therapeutic setting.

As stated above, Gas6 is a ligand for MerTK and increases pAKT activation upon interaction with MerTK. The present studies also compared the effect of monovalent versus bivalent anti-MerTK antibodies of the present disclosure on pAKT in the presence of MerTK ligand Gas6. FIG. 8 below shows IC50 values of huGas6-mediated pAKT reducing activity by monovalent and bivalent anti-MerTK antibodies of the present disclosure. As shown in Table 8, monovalent anti-MerTK antibodies MTK-16.2 and MTK-33.11 showed higher IC50 values than their bivalent counterparts on affecting pAKT activity in the presence of Gas6. Monovalent anti-MerTK antibodies MTK-16.2 and MTK-33.11 reduced huGas6-mediated (e.g., ligand dependent) pAKT activity with IC50 values in the range of approximately 15.35 nM to 17.85 nM as measured in this assay; whereas bispecific anti-MerTK:anti-PDL1 antibodies of the present disclosure reduced ligand-dependent pAKT activity with IC50 values in the range of approximately 12.26 nM to 9.027 nM. These data indicated that bivalent anti-MerTK antibodies of the present disclosure are more effective at reducing Gas6-mediated pAKT activity compared to monovalent and bispecific anti-MerTK:anti-PDL1 antibodies.

TABLE 8

| | Average |
|---|---|
| | IC50 (nM) |
| Antibody | in the presence of huGas6 |
| MTK-16.2 monovalent | 15.35 |
| MTK-16.2:anti-PDL1 bispecific | 12.26 |
| MTK-16.2 bivalent | 3.218 |
| MTK-33.11 monovalent | 17.85 |
| MTK-33.11:anti-PDL1 bispecific | 9.027 |
| MTK-33.11 bivalent | 1.248 |

### Example 14: Binding Kinetics of Bispecific Anti-MerTK:Anti-PDL1 Antibodies

Binding kinetics of bispecific anti-MerTK:anti-PDL1 antibodies of the present disclosure to human, cyno, and murine MerTK were evaluated using a Carterra LSA instrument. Briefly, anti-MerTK antibodies were prepared by diluting to 10 μg/ml in 10 mM Acetate, pH 4.25 (Carterra). A HC30 sensor chip (Carterra) was activated using the single channel flow cell with a 7-minute injection of a 1:1:1 mixture of 100 mM MES pH 5.5, 100 mM sulfo-NHS, 400 mM EDC (all reconstituted in MES pH 5.5; 100 μl of each mixed in vial immediately before running assay). After switching to the multi-channel array flow cell, the antibodies were injected over the activated chip in a 96-spot array for 15 minutes. A duplicate injection of the antibodies on a second block of the chip was spotted as before. The remaining unconjugated active groups on the chip were then blocked by injecting 1M Ethanolamine pH 8.5 (Carterra) for 7 minutes using the single channel flow cell.

After priming with running buffer (HBS-TE, Carterra) with 0.5 mg/ml BSA (Sigma), the immobilized anti-MerTK antibodies were tested for their ability to bind to several forms of recombinant MerTK extracellular domain, including that of human, cynomolgus, and mouse orthologs as described above. Estimates of affinity were generated by injecting each analyte over the entire antibody array using the single channel flow cell. Six 3-fold serial dilutions of MerTK analytes (human MerTK 1.2 mM-4.9 nM; cyno and mouse MerTK 3-1.2 nM) were prepared in running buffer, and injected for 300 seconds in serial from lowest to highest concentration. Dissociation was followed for 300 seconds before regenerating after each injection with 2×30 seconds of 10 mM Glycine pH2.5. Three buffer blanks were run between each series (one species per series). After all concentrations of all three species of MerTK were injected, a duplicate set of injections was performed, so that each concentration for all three species was injected in widely spaced duplicates. Data were processed and analyzed using NextGenKIT high-throughput kinetics analysis software (Carterra). Duplicate injections overlaid nearly perfectly for all samples, indicating that the surface was not degraded during the run.

The equilibrium dissociation constants (K_{D}) were then calculated from the fitted association and dissociation rate constants (k-on and k-off) for anti-MerTK antibodies of the present disclosure. In general, the association profiles of the antibodies were complex, and did not fit ideally to a 1:1 model; therefore, the K_{D} values are summarized in Table 9 below represents estimates for comparison between antibodies and antigens.

TABLE 9

| | K_{D} (nM) | | |
|---|---|---|---|
| Antibody | huMerTK | moMerTK | cynoMerTK |
| MTK-16.2 monovalent | 50 | No binding | 60 |
| | | detected | |
| MTK-16.2:anti-PDL1 | 30 | No binding | 30 |
| bispecific | | detected | |
| MTK-16.2 bivalent | 50 | No binding | 60 |
| | | detected | |
| MTK-33.11 monovalent | 2 | 40 | 3 |
| MTK-33.11:anti-PDL1 | 2 | 40 | 2 |
| bispecific | | | |
| MTK-33.11 full IgG | 2 | 50 | 2 |

As shown in Table 9, No affinity differences were observed for monovalent or bivalent anti-MerTK antibodies tested, indicating monovalent and bivalent anti-MerTK antibodies maintain monovalent binding characteristics.

### Example 15: The Effect of Bivalent Anti-MerTK Antibody Treatment in MC38 Mouse Tumor Model

The ability of bivalent anti-MerTK antibodies of the present disclosure to delay MC38 tumor growth in vivo was examined as follows. MC38 cells were implanted subcutaneously on huMerTK Knock-In (KI) mice. When tumor size reached approximately 80-100 mm³ in volume, mice were treated with 10 mg/kg of bivalent anti-MerTK antibody MTK-33.11 plus 3 mg/kg of bivalent anti-PDL1 antibody, bivalent anti-MerTK antibody MTK-16.2 plus 3 mg/kg bivalent anti-PDL1 antibody, 3 mg/kg of anti-PDL1 alone, or 10 mg/kg of control antibody twice a week for three weeks. Tumor volume was measured three times a week.

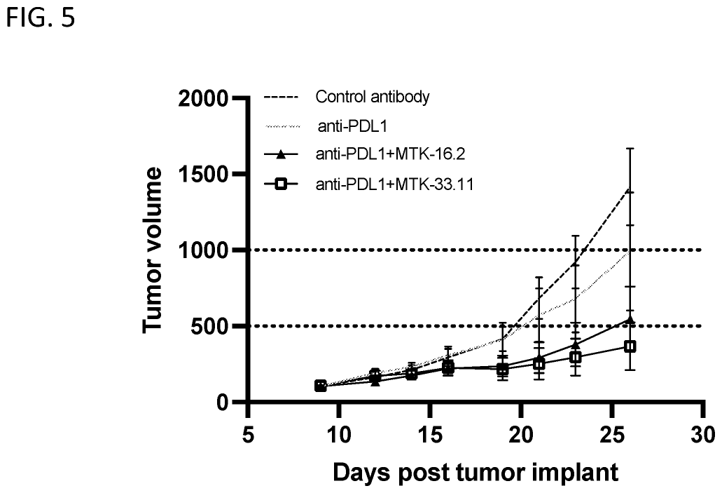

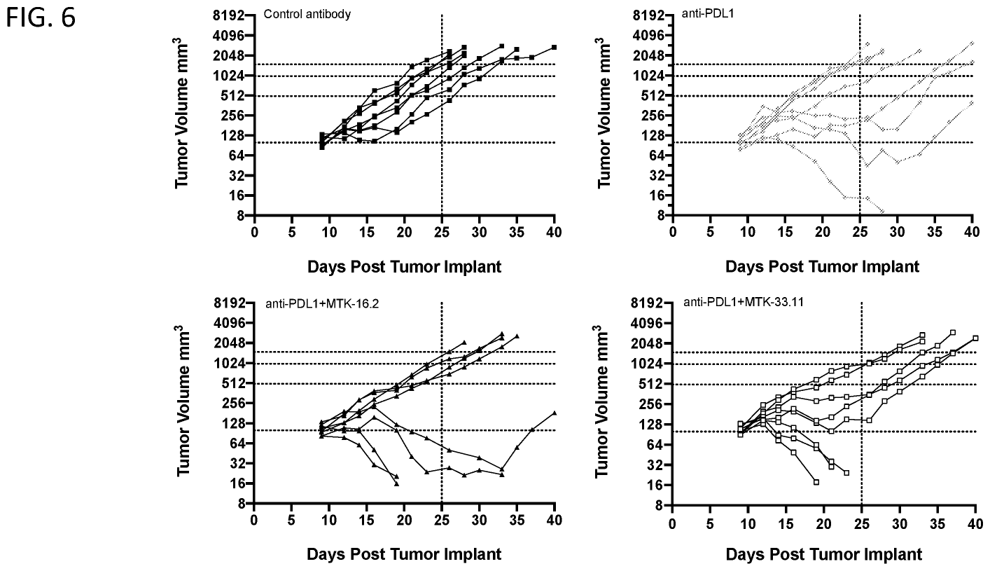

FIG. 5 shows that combination of bivalent anti-PDL1 antibody with either bivalent anti-MerTK MTK-33.11 antibody or bivalent anti-MerTK antibody MTK-16.2 reduced the tumor growth rate (as measured by changes in tumor volume over time) in mice compared to that observed in mice treated with bivalent anti-PDL1 antibody alone. FIG. 6 shows the tumor growth of individual mice in each group (control antibody, bivalent anti-PDL1 antibody, bivalent anti-PLD1 antibody+bivalent anti-MerTK antibody MTK-33.11, and bivalent anti-PLD1 antibody+bivalent anti-MerTK antibody MTK-16.2. These results show that bivalent anti-MerTK antibodies of the present disclosure in combination with bivalent anti-PDL1 antibody are more effective at in inhibiting tumor growth compared to that observed with bivalent anti-PLD1 antibody alone.

### Example 16: Certain Bispecific Anti-MerTK:Anti-PDL1 Antibody Configurations

Table 10 and Table 11 below provide certain bivalent anti-MerTK:anti-PDL1 antibody configurations of the present disclosure.

TABLE 10
Anti-MerTK 16.2:Anti-PDL1

| Fc configuration | 16.2 heavy chain | 16.2 light chain | PDL1 heavy chain | PDL1 light chain |
|---|---|---|---|---|
| IgG1 wildtype Fc | SEQ ID NO: 17 | SEQ ID NO: 21 | SEQ ID NO: 27 | SEQ ID NO: 31 |
| IgG1 LAPAPS Fc | SEQ ID NO: 18 | SEQ ID NO: 21 | SEQ ID NO: 28 | SEQ ID NO: 31 |
| IgG1 NSLF Fc | SEQ ID NO: 19 | SEQ ID NO: 21 | SEQ ID NO: 29 | SEQ ID NO: 31 |
| IgG4 Fc | SEQ ID NO: 20 | SEQ ID NO: 21 | SEQ ID NO: 30 | SEQ ID NO: 31 |

TABLE 11
Anti-MerTK 33.11:Anti-PDL1

| Fc configuration | 33.11 heavy chain | 33.11 light chain | PDL1 heavy chain | PDL1 light chain |
|---|---|---|---|---|
| IgG1 wildtype Fc | SEQ ID NO: 22 | SEQ ID NO: 26 | SEQ ID NO: 27 | SEQ ID NO: 31 |
| IgG1 LAPAPS Fc | SEQ ID NO: 23 | SEQ ID NO: 26 | SEQ ID NO: 28 | SEQ ID NO: 31 |
| IgG1 NSLF Fc | SEQ ID NO: 24 | SEQ ID NO: 26 | SEQ ID NO: 29 | SEQ ID NO: 31 |
| IgG4 Fc | SEQ ID NO: 25 | SEQ ID NO: 26 | SEQ ID NO: 30 | SEQ ID NO: 31 |

Table 12 and Table 13 below provide certain bispecific anti-MerTK:anti-PDL1 antibody configurations comprising heavy chain “knob” amino acid substitutions within the Fc region of the anti-MerTK heavy chain arm, and comprising heavy chain “hole” amino acid substitutions within the Fc region of the anti-PDL1 heavy chain arm.

TABLE 12
Anti-MerTK 16.2 (knob):Anti-PDL1 (hole)

| | 16.2 heavy | | PDL1 heavy | |
|---|---|---|---|---|
| Fc configuration | chain (knob) | 16.2 light chain | chain (hole) | PDL1 light chain |
| IgG1 wildtype Fc | SEQ ID NO: 32 | SEQ ID NO: 21 | SEQ ID NO: 42 | SEQ ID NO: 31 |
| IgG1 LAPAPS Fc | SEQ ID NO: 33 | SEQ ID NO: 21 | SEQ ID NO: 43 | SEQ ID NO: 31 |
| IgG1 NSLF Fc | SEQ ID NO: 34 | SEQ ID NO: 21 | SEQ ID NO: 44 | SEQ ID NO: 31 |
| IgG4 Fc | SEQ ID NO: 35 | SEQ ID NO: 21 | SEQ ID NO: 45 | SEQ ID NO: 31 |
| IgG4 S228P Fc | SEQ ID NO: 36 | SEQ ID NO: 21 | SEQ ID NO: 46 | SEQ ID NO: 31 |

TABLE 13
Anti-MerTK 33.11 (knob):Anti-PDL1 (hole)

| | 33.11 heavy | | PDL1 heavy | |
|---|---|---|---|---|
| Fc configuration | chain (knob) | 33.11 light chain | chain (hole) | PDL1 light chain |
| IgG1 wildtype Fc | SEQ ID NO: 37 | SEQ ID NO: 26 | SEQ ID NO: 42 | SEQ ID NO: 31 |
| IgG1 LAPAPS Fc | SEQ ID NO: 38 | SEQ ID NO: 26 | SEQ ID NO: 43 | SEQ ID NO: 31 |
| IgG1 NSLF Fc | SEQ ID NO: 39 | SEQ ID NO: 26 | SEQ ID NO: 44 | SEQ ID NO: 31 |
| IgG4 Fc | SEQ ID NO: 40 | SEQ ID NO: 26 | SEQ ID NO: 45 | SEQ ID NO: 31 |
| IgG4 S228P Fc | SEQ ID NO: 41 | SEQ ID NO: 26 | SEQ ID NO: 46 | SEQ ID NO: 31 |

What is claimed is:

1. A bispecific antibody that binds to human Mer Tyrosine Kinase (MerTK) and programmed death-ligand 1 (PDL1), wherein the bispecific antibody contains a first antigen-binding domain that binds to human MerTk and a second antigen-binding domain that binds to PDL1.

2. The antibody of claim 1, wherein the first antigen-binding domain binds to the Ig1 domain of MerTK protein.

3. The bispecific antibody of claim 1 or 2, wherein the first antigen binding domain competitively inhibits binding to the MerTK of an antibody comprising a variable heavy chain comprising the amino acid sequence of SEQ ID NO:9 and a variable light chain comprising the amino acid sequence of SEQ ID NO:10.

4. The bispecific antibody of any one of claims 1-3, wherein the first antigen binding domain binds to the same MerTK epitope as an antibody comprising a variable heavy chain comprising the amino acid sequence of SEQ ID NO:9 and a variable light chain comprising the amino acid sequence of SEQ ID NO:10.

5. The bispecific antibody of any one of claims 1-4, wherein the first antigen binding domain an HVR-H1 comprising amino acids 31-35 of SEQ ID NO:9, an HVR-H2 comprising amino acids 50-66 of SEQ ID NO:9, an HVR-H3 comprising amino acids 99-109 of SEQ ID NO:9, an HVR-L1 comprising amino acids 24-34 of SEQ ID NO:10, an HVR-L2 comprising amino acids 50-56 of SEQ ID NO:10, and an HVR-L3 comprising amino acids 89-97 of SEQ ID NO:10.

6. The bispecific antibody of any one of claims 1-4, wherein the first antigen binding domain comprises the HVRs of the 16.2 antibody, optionally wherein the HVRs are the Kabat-defined HVRs, the Chothia-defined HVRs, the AbM-defined HVRs, or the contact-defined HVRs

7. The bispecific antibody of any one of claims 1-6, wherein the first antigen binding domain comprises a variable heavy chain comprising an amino acid sequence that is at least 85%, at least 90%, or at least 95% identical to the amino acid sequence of SEQ ID NO:9.

8. The bispecific antibody of any one of claims 1-7, wherein the first antigen binding domain comprises a variable light chain comprising an amino acid sequence that is at least 85%, at least 90%, or at least 95% identical to the amino acid sequence of SEQ ID NO:10.

9. The bispecific antibody of any one of claims 1-8, wherein the first antigen binding domain comprises a variable heavy chain comprising the amino acid sequence of SEQ ID NO:9 and/or a variable light chain comprising the amino acid sequence of SEQ ID NO:10.

10. The bispecific antibody of claim 1 or 2, wherein the first antigen binding domain binds to the same MerTK epitope as an antibody comprising a variable heavy chain comprising the amino acid sequence of SEQ ID NO: 13 and a variable light chain comprising the amino acid sequence of SEQ ID NO: 14.

11. The bispecific antibody of any one of claims 1, 2 and 10, wherein the first antigen binding domain competitively inhibits binding to the MerTK of an antibody comprising a variable heavy chain comprising the amino acid sequence of SEQ ID NO: 13 and a variable light chain comprising the amino acid sequence of SEQ ID NO:14.

12. The bispecific antibody of any one of claims 1, 2, 10, and 11, wherein the first antigen binding domain comprises an HVR-H1 comprising amino acids 31-35 of SEQ ID NO:13, an HVR-H2 comprising amino acids 50-66 of SEQ ID NO:13, an HVR-H3 comprising amino acids 99-108 of SEQ ID NO:13, an HVR-L1 comprising amino acids 24-34 of SEQ ID NO:14, an HVR-L2 comprising amino acids 50-56 of SEQ ID NO:14, and an HVR-L3 comprising amino acids 89-97 of SEQ ID NO: 14.

13. The bispecific antibody of any one of claims 1, 2, 10 and 11, wherein the first antigen binding domain comprises the HVRs of the 13.11 antibody, optionally wherein the HVRs are the Kabat-defined HVRs, the Chothia-defined HVRs, the AbM-defined HVRs, or the contact-defined HVRs.

14. The bispecific antibody of any one of claims 1, 2, and 10-13, wherein the first antigen binding domain comprises a variable heavy chain comprising an amino acid sequence that is at least 85%, at least 90%, or at least 95% identical to the amino acid sequence of SEQ ID NO:13.

15. The bispecific antibody of any one of claims 1, 2, and 10-14, wherein the first antigen binding domain comprises a variable light chain comprising an amino acid sequence that is at least 85%, at least 90%, or at least 95% identical to the amino acid sequence of SEQ ID NO: 14.

16. The bispecific antibody of any one of claims 1, 2, and 10-15, wherein the first antigen binding domain comprises a variable heavy chain comprising the amino acid sequence of SEQ ID NO:13 and a variable light chain comprising the amino acid sequence of SEQ ID NO:14.

17. The bispecific antibody of any one of claims 1-16, wherein the second antigen binding domain binds to the same PDL1 epitope as an antibody comprising a variable heavy chain comprising the amino acid sequence of SEQ ID NO:52 and a variable light chain comprising the amino acid sequence of SEQ ID NO:53.

18. The bispecific antibody of any one of claims 1-17, wherein the second antigen binding domain competitively inhibits binding to the PDL1 of an antibody comprising a variable heavy chain comprising the amino acid sequence of SEQ ID NO:52 and a variable light chain comprising the amino acid sequence of SEQ ID NO:53.

19. The bispecific antibody of any one of claims 1-18, wherein the second antigen binding domain comprises an HVR-H1 comprising amino acids 31-35 of SEQ ID NO:52, an HVR-H2 comprising amino acids 50-66 of SEQ ID NO:52, an HVR-H3 comprising amino acids 99-107 of SEQ ID NO:52, an HVR-L1 comprising amino acids 24-34 of SEQ ID NO:53, an HVR-L2 comprising amino acids 50-56 of SEQ ID NO:53, and an HVR-L3 comprising amino acids 89-97 of SEQ ID NO:53

20. The bispecific antibody of any one of claims 1-18, wherein the second antigen binding domain comprises the HVRs of the atezolizumab antibody, optionally wherein the HVRs are the Kabat-defined HVRs, the Chothia-defined HVRs, the AbM-defined HVRs, or the contact-defined HVRs.

21. The bispecific antibody of any one of claims 1-20, wherein the second antigen binding domain comprises a variable heavy chain comprising an amino acid sequence that is at least 85%, at least 90%, or at least 95% identical to the amino acid sequence of SEQ ID NO:52.

22. The bispecific antibody of any one of claims 1-21, wherein the second antigen binding domain comprises a variable light chain comprising an amino acid sequence that is at least 85%, at least 90%, or at least 95% identical to the amino acid sequence of SEQ ID NO:53.

23. The bispecific antibody of any one of claims 1-22, wherein the second antigen binding domain comprises a variable heavy chain comprising the amino acid sequence of SEQ ID NO:52 and a variable light chain comprising the amino acid sequence of SEQ ID NO:53.

24. The bispecific antibody of any one of claims 1-23, wherein the bispecific antibody is of the IgG class, the IgM class, or the IgA class.

25. The bispecific antibody of claim 24, wherein the bispecific antibody is of the IgG class, optionally wherein the bispecific antibody has an IgG1, an IgG2, or an IgG4 isotype.

26. The bispecific antibody of claim 25, wherein the bispecific antibody is an IgG1 antibody.

27. The bispecific antibody of claim 25, wherein the bispecific antibody is an IgG4 antibody.

28. The bispecific antibody of any one of claims 1-27, wherein the bispecific antibody (i) has two arms comprising different antigen-binding domains, (ii) is a single chain antibody that has specificity to two different epitopes, (iii) is a chemically-linked bispecific (Fab′)2 fragment, (iv) is a fusion of two single chain diabodies resulting in a tetravalent bispecific antibody that has two binding sites for each of the target antigens, (v) is a combination of scFvs with a diabody resulting in a multivalent molecule, (vi) comprises two scFvs fused to both termini of a human Fab-arm, or (vii) is a diabody.

29. The bispecific antibody of any one of claims 1-27, wherein the bispecific antibody is a kappa-lambda body, a dual-affinity re-targeting molecule (DART), a knob-in-hole antibody, a strand-exchange engineered domain body (SEEDbody), or a DuoBody.

30. The bispecific antibody of any one of claims 1-29, wherein the bispecific antibody comprises an Fc region comprising a first polypeptide and a second polypeptide wherein:
(a) the first polypeptide comprises the amino acid substitution T366Y, and the second polypeptide comprises the amino acid substation Y407T;
(b) the first polypeptide comprises the amino acid substitution T366W, and the second polypeptide comprises the amino acid substitutions T366S, L368W, and Y407V;
(c) the first polypeptide comprises the amino acid substitution T366W, and the second polypeptide comprises the amino acid substitutions T366S, L368A, and Y407V;
(d) the first polypeptide comprises the amino acid substitutions T366W, and S354C and the second polypeptide comprises the amino acid substitutions T366S, L368A, Y407V, and Y349C;
(e) the first polypeptide comprises the amino acid substitutions T350V, L351Y, F405A, Y407V, and the second polypeptide comprises the amino acid substitutions T350V, T366L, K392L, and T394W;
(f) the first polypeptide comprises the amino acid substitutions K360D, D399M, and Y407A, and the second polypeptide comprises the amino acid substitutions E345R, Q347R, T366V, and K409V;
(g) the first polypeptide comprises the amino acid substitutions K409D and K392D, and the second polypeptide comprises the amino acid substitutions D399K and E356K;
(h) the first polypeptide comprises the amino acid substitutions K360E and K409W, and the second polypeptide comprises the amino acid substitutions Q347R, D399V, and F405T;
(i) the first polypeptide comprises the amino acid substitutions L360E, K409W, and Y349C, and the second polypeptide comprises the amino acid substitutions Q347R, D399V, F405T, and S354C; or
(j) the first polypeptide comprises the amino acid substitutions K370E and K409W, and the second polypeptide comprises the amino acid substitutions E357N, D399V, and F405T;
wherein the substitution is according to EU numbering.

31. The bispecific antibody of any one of claims 1-30, wherein the bispecific antibody comprises a knob mutation and a hole mutation.

32. The bispecific antibody of claim 31, wherein the knob mutation comprises the amino acid substitution T366W according to EU numbering.

33. The bispecific antibody of claim 31 or 32, wherein the hole mutation comprises the amino acids substitutions T366S, L368A, and Y407V according to EU numbering.

34. The bispecific antibody of any one of claims 1-33, wherein the bispecific antibody comprises an Fc region comprising an amino acid substitution, addition, or deletion that promotes heterodimerization.

35. The bispecific antibody of any one of claims 1-25, and 27-34, wherein the bispecific antibody comprises the amino acid substitution S228P according to EU numbering.

36. The bispecific antibody of any one of claims 1-35, wherein the bispecific antibody comprises the amino acid substitutions L234A, L235A, and P331S (LALAPS) accordingly to EU numbering.

37. The bispecific antibody of any one of claims 1-36, wherein the bispecific antibody comprises the amino acid substitutions N325S and L328F (NSLF) according to EU numbering.

38. The bispecific antibody of any one of claims 1-9, 17-26, 28, and 29, wherein the bispecific antibody comprises a heavy chain comprising amino acids 1-449 of SEQ ID NO:17 or the amino acid sequence of SEQ ID NO:17.

39. The bispecific antibody of any one of claims 1-9, 17-26, 28, 29, and 36, wherein the bispecific antibody comprises a heavy chain comprising amino acids 1-449 of SEQ ID NO:18 or the amino acid sequence of SEQ ID NO: 18.

40. The bispecific antibody of any one of claims 1-9, 17-26, 28, 29, and 37, wherein the bispecific antibody comprises a heavy chain comprising amino acids 1-449 of SEQ ID NO: 19 or the amino acid sequence of SEQ ID NO: 19.

41. The bispecific antibody of any one of claims 1-9, 17-25, 27, 28, and 29, wherein the bispecific antibody comprises a heavy chain comprising amino acids 1-446 of SEQ ID NO:20 or the amino acid sequence of SEQ ID NO:20.

42. The bispecific antibody of any one of claims 1-9, 17-26, and 28-34, wherein the bispecific antibody comprises a heavy chain comprising amino acids 1-453 of SEQ ID NO:32 or the amino acid sequence of SEQ ID NO:32.

43. The bispecific antibody of any one of claims 1-9, 17-26, 28-34, and 36, wherein the bispecific antibody comprises a heavy chain comprising amino acids 1-449 of SEQ ID NO:33 or the amino acid sequence of SEQ ID NO:33.

44. The bispecific antibody of any one of claims 1-9, 17-26, 28-34, and 37, wherein the bispecific antibody comprises a heavy chain comprising amino acids 1-449 of SEQ ID NO:34 or the amino acid sequence of SEQ ID NO:34.

45. The bispecific antibody of any one of claims 1-9, 17-25, and 27-34, wherein the bispecific antibody comprises a heavy chain comprising amino acids 1-446 of SEQ ID NO:35 or the amino acid sequence of SEQ ID NO:35.

46. The bispecific antibody of any one of claims 1-9, 17-25, and 27-35, wherein the bispecific antibody comprises a heavy chain comprising amino acids 1-446 of SEQ ID NO:36 or the amino acid sequence of SEQ ID NO:36.

47. The bispecific antibody of any one of claims 1-9, 17-26, 28, 29, 36 and 37, wherein the bispecific antibody comprises a light chain comprising the amino acid sequence of SEQ ID NO:21.

48. The bispecific antibody any one of claims 1, 2, 10-26, 28, and 29, wherein the bispecific antibody comprises a heavy chain comprising amino acids 1-448 of SEQ ID NO:22 or the amino acid sequence of SEQ ID NO:22.

49. The bispecific antibody of any one of claims 1, 2, 10-26, 28, 29, and 36, wherein the bispecific antibody comprises a heavy chain comprising amino acids 1-448 of SEQ ID NO:23 or the amino acid sequence of SEQ ID NO:23.

50. The bispecific antibody of any one of claims 1, 2, 10-26, 28, 29, and 37, wherein the bispecific antibody comprises a heavy chain comprising amino acids 1-448 of SEQ ID NO:24 or the amino acid sequence of SEQ ID NO:24.

51. The bispecific antibody of any one of claims 1, 2, 10-25, and 27-29, wherein the bispecific antibody comprises a heavy chain comprising amino acids 1-445 of SEQ ID NO:25 or the amino acid sequence of SEQ ID NO:25.

52. The bispecific antibody of any one of claims 1, 2, 10-26, and 28-34, wherein the bispecific antibody comprises a heavy chain comprising amino acids 1-448 of SEQ ID NO:37 or the amino acid sequence of SEQ ID NO:37.

53. The bispecific antibody of any one of claims 1, 2, 10-26, 28-34, and 36, wherein the bispecific antibody comprises a heavy chain comprising amino acids 1-448 of SEQ ID NO:38 or the amino acid sequence of SEQ ID NO:38.

54. The bispecific antibody of any one of claims 1, 2, 10-26, 28-34, and 37, wherein the bispecific antibody comprises a heavy chain comprising amino acids 1-448 of SEQ ID NO:39 or the amino acid sequence of SEQ ID NO:39.

55. The bispecific antibody of any one of claims 1, 2, 10-25, and 27-34, wherein the bispecific antibody comprises a heavy chain comprising amino acids 1-445 of SEQ ID NO:40 or the amino acid sequence of SEQ ID NO:40.

56. The bispecific antibody of any one of claims 1, 2, 10-25, and 27-35, wherein the bispecific antibody comprises a heavy chain comprising amino acids 1-445 of SEQ ID NO:41 or the amino acid sequence of SEQ ID NO:41.

57. The bispecific antibody of any one of claims 1, 2, 10-26, 28, 29, 36, and 37, wherein the bispecific antibody comprises a light chain comprising the amino acid sequence of SEQ ID NO:26.

58. The bispecific antibody of any one of claims 1-26, 28, and 29, wherein the bispecific antibody comprises a heavy chain comprising amino acids 1-447 of SEQ ID NO:27 or the amino acid sequence of SEQ ID NO:27.

59. The bispecific antibody of any one of claims 1-26, 28, 29, and 36, wherein the bispecific antibody comprises a heavy chain comprising amino acids 1-447 of SEQ ID NO:28 or the amino acid sequence of SEQ ID NO:28.

60. The bispecific antibody of any one of claims 1-26, 28, 29, and 37, wherein the bispecific antibody comprises a heavy chain comprising amino acids 1-447 of SEQ ID NO:29 or the amino acid sequence of SEQ ID NO:29.

61. The bispecific antibody of any one of claims 1-25 and 27-29, wherein the bispecific antibody comprises a heavy chain comprising amino acids 1-444 of SEQ ID NO:30 or the amino acid sequence of SEQ ID NO:30.

62. The bispecific antibody of any one of claims 1-26 and 28-34, wherein the bispecific antibody comprises a heavy chain comprising amino acids 1-447 of SEQ ID NO:42 or the amino acid sequence of SEQ ID NO:42.

63. The bispecific antibody of any one of claims 1-26, 28-34, and 36, wherein the bispecific antibody comprises a heavy chain comprising amino acids 1-447 of SEQ ID NO:43 or the amino acid sequence of SEQ ID NO:43.

64. The bispecific antibody of any one of claims 1-26, 28-34, and 37, wherein the bispecific antibody comprises a heavy chain comprising amino acids 1-447 of SEQ ID NO:44 or the amino acid sequence of SEQ ID NO:44.

65. The bispecific antibody of any one of claims 1-25 and 27-34, wherein the bispecific antibody comprises a heavy chain comprising amino acids 1-444 of SEQ ID NO:45 or the amino acid sequence of SEQ ID NO:45.

66. The bispecific antibody of any one of claims 1-25 and 27-35, wherein the bispecific antibody comprises a heavy chain comprising amino acids 1-444 of SEQ ID NO:46 or the amino acid sequence of SEQ ID NO:46.

67. The bispecific antibody of any one of claims 1-26, 28, 29, 36, and 37, wherein the bispecific antibody comprises a light chain comprising the amino acid sequence of SEQ ID NO:31.

68. The bispecific antibody of any one of claims 1-26, 28-34, 36-37, 47, 57, and 67, wherein the bispecific antibody comprises a heavy chain constant region comprising the amino acid sequence of SEQ ID NO:47.

69. The bispecific antibody of any one of claims 1-26, 28-34, 36-37, 47, 57, and 67, wherein the bispecific antibody comprises a heavy chain constant region comprising the amino acid sequence of SEQ ID NO:48.

70. The bispecific antibody of any one of claims 1-26, 28-34, 36-37, 47, 57, and 67, wherein the bispecific antibody comprises a heavy chain constant region comprising the amino acid sequence of SEQ ID NO:49.

71. The bispecific antibody of any one of claims 1-26, 28-34, 36-37, 47, 57, and 67, wherein the bispecific antibody comprises a heavy chain constant region comprising the amino acid sequence of SEQ ID NO:50.

72. The bispecific antibody of any one of claims 1-26, 28-34, 36-37, 47, 57, and 67, wherein the bispecific antibody comprises a heavy chain constant region comprising the amino acid sequence of SEQ ID NO:51.

73. The bispecific antibody of any one of claims 1-9, 17-26, 28, and 29, wherein the bispecific antibody comprises a heavy chain comprising amino acids 1-449 of SEQ ID NO:17 or the amino acid sequence of SEQ ID NO:17, a light chain comprising the amino acid sequence of SEQ ID NO:21, a heavy chain comprising amino acids 1-447 of SEQ ID NO:27 or the amino acid sequence of SEQ ID NO:27 and a light chain comprising the amino acid sequence of SEQ ID NO:31.

74. The bispecific antibody of any one of claims 1-9, 17-26, 28, and 29, wherein the bispecific antibody comprises a heavy chain comprising amino acids 1-449 of SEQ ID NO:18 or the amino acid sequence of SEQ ID NO:18, a light chain comprising the amino acid sequence of SEQ ID NO:21, a heavy chain comprising amino acids 1-447 of SEQ ID NO:28 or the amino acid sequence of SEQ ID NO:28 and a light chain comprising the amino acid sequence of SEQ ID NO:31.

75. The bispecific antibody of any one of claims 1-9, 17-26, 28, and 29, wherein the bispecific antibody comprises a heavy chain comprising amino acids 1-449 of SEQ ID NO: 19 or the amino acid sequence of SEQ ID NO: 19, a light chain comprising the amino acid sequence of SEQ ID NO:21, a heavy chain comprising amino acids 1-447 of SEQ ID NO:29 or the amino acid sequence of SEQ ID NO:29 and a light chain comprising the amino acid sequence of SEQ ID NO:31.

76. The bispecific antibody of any one of claims 1-9, 17-25, and 27-29, wherein the bispecific antibody comprises a heavy chain comprising amino acids 1-446 of SEQ ID NO:20 or the amino acid sequence of SEQ ID NO:20, a light chain comprising the amino acid sequence of SEQ ID NO:21, a heavy chain comprising amino acids 1-444 of SEQ ID NO:30 or the amino acid sequence of SEQ ID NO:30 and a light chain comprising the amino acid sequence of SEQ ID NO:31.

77. The bispecific antibody of any one of claims 1, 2, 10-26, 28, and 29, wherein the bispecific antibody comprises a heavy chain comprising amino acids 1-448 of SEQ ID NO:22 or the amino acid sequence of SEQ ID NO:22, a light chain comprising the amino acid sequence of SEQ ID NO:26, a heavy chain comprising amino acids 1-447 of SEQ ID NO:27 or the amino acid sequence of SEQ ID NO:27 and a light chain comprising the amino acid sequence of SEQ ID NO:31.

78. The bispecific antibody of any one of claims 1, 2, 10-26, 28, and 29, wherein the bispecific antibody comprises a heavy chain comprising amino acids 1-448 of SEQ ID NO:23 or the amino acid sequence of SEQ ID NO:23, a light chain comprising the amino acid sequence of SEQ ID NO:26, a heavy chain comprising amino acids 1-447 of SEQ ID NO:28 or the amino acid sequence of SEQ ID NO:28 and a light chain comprising the amino acid sequence of SEQ ID NO:31.

79. The bispecific antibody of any one of claims 1, 2, 10-26, 28, and 29, wherein the bispecific antibody comprises a heavy chain comprising amino acids 1-448 of SEQ ID NO:24 or the amino acid sequence of SEQ ID NO:24, a light chain comprising the amino acid sequence of SEQ ID NO:26, a heavy chain comprising amino acids 1-447 of SEQ ID NO:29 or the amino acid sequence of SEQ ID NO:29 and a light chain comprising the amino acid sequence of SEQ ID NO:31.

80. The bispecific antibody of any one of claims 1, 2, 10-25, and 27-29, wherein the bispecific antibody comprises a heavy chain comprising amino acids 1-445 of SEQ ID NO:25 or the amino acid sequence of SEQ ID NO:25, a light chain comprising the amino acid sequence of SEQ ID NO:26, a heavy chain comprising amino acids 1-444 of SEQ ID NO:30 or the amino acid sequence of SEQ ID NO:30 and a light chain comprising the amino acid sequence of SEQ ID NO:31.

81. The bispecific antibody of any one of claims 1-9, 17-26, 28, and 29, wherein the bispecific antibody comprises a heavy chain comprising amino acids 1-453 of SEQ ID NO:32 or the amino acid sequence of SEQ ID NO:32, a light chain comprising the amino acid sequence of SEQ ID NO:21, a heavy chain comprising amino acids 1-447 of SEQ ID NO:42 or the amino acid sequence of SEQ ID NO:42 and a light chain comprising the amino acid sequence of SEQ ID NO:31.

82. The bispecific antibody of any one of claims 1-9, 17-26, 28, and 29, wherein the bispecific antibody comprises a heavy chain comprising amino acids 1-449 of SEQ ID NO:33 or the amino acid sequence of SEQ ID NO:33, a light chain comprising the amino acid sequence of SEQ ID NO:21, a heavy chain comprising amino acids 1-447 of SEQ ID NO:43 or the amino acid sequence of SEQ ID NO:43 and a light chain comprising the amino acid sequence of SEQ ID NO:31.

83. The bispecific antibody of any one of claims 1-9, 17-26, 28, and 29, wherein the bispecific antibody comprises a heavy chain comprising amino acids 1-449 of SEQ ID NO:34 or the amino acid sequence of SEQ ID NO:34, a light chain comprising the amino acid sequence of SEQ ID NO:21, a heavy chain comprising amino acids 1-447 of SEQ ID NO:44 or the amino acid sequence of SEQ ID NO:44 and a light chain comprising the amino acid sequence of SEQ ID NO:31.

84. The bispecific antibody of any one of claims 1-9, 17-25, and 27-29, wherein the bispecific antibody comprises a heavy chain comprising amino acids 1-446 of SEQ ID NO:35 or the amino acid sequence of SEQ ID NO:35, a light chain comprising the amino acid sequence of SEQ ID NO:21, a heavy chain comprising amino acids 1-444 of SEQ ID NO:45 or the amino acid sequence of SEQ ID NO:45 and a light chain comprising the amino acid sequence of SEQ ID NO:31.

85. The bispecific antibody of any one of claims 1-9, 17-25, and 27-29, wherein the bispecific antibody comprises a heavy chain comprising amino acids 1-446 of SEQ ID NO:36 or the amino acid sequence of SEQ ID NO:36, a light chain comprising the amino acid sequence of SEQ ID NO:21, a heavy chain comprising amino acids 1-444 of SEQ ID NO:46 or the amino acid sequence of SEQ ID NO:46 and a light chain comprising the amino acid sequence of SEQ ID NO:31.

86. The bispecific antibody of any one of claims 1, 2, 10-26, 28, and 29, wherein the bispecific antibody comprises a heavy chain comprising amino acids 1-448 of SEQ ID NO:37 or the amino acid sequence of SEQ ID NO:37, a light chain comprising the amino acid sequence of SEQ ID NO:26, a heavy chain comprising amino acids 1-447 of SEQ ID NO:42 or the amino acid sequence of SEQ ID NO:42 and a light chain comprising the amino acid sequence of SEQ ID NO:31.

87. The bispecific antibody of any one of claims 1, 2, 10-26, 28, and 29, wherein the bispecific antibody comprises a heavy chain comprising amino acids 1-448 of SEQ ID NO:38 or the amino acid sequence of SEQ ID NO:38, a light chain comprising the amino acid sequence of SEQ ID NO:26, a heavy chain comprising amino acids 1-447 of SEQ ID NO:43 or the amino acid sequence of SEQ ID NO:43 and a light chain comprising the amino acid sequence of SEQ ID NO:31.

88. The bispecific antibody of any one of claims 1, 2, 10-26, 28, and 29, wherein the bispecific antibody comprises a heavy chain comprising amino acids 1-448 of SEQ ID NO:39 or the amino acid sequence of SEQ ID NO:39, a light chain comprising the amino acid sequence of SEQ ID NO:26, a heavy chain comprising amino acids 1-447 of SEQ ID NO:44 or the amino acid sequence of SEQ ID NO:44 and a light chain comprising the amino acid sequence of SEQ ID NO:31.

89. The bispecific antibody of any one of claims 1, 2, 10-25, and 27-29, wherein the bispecific antibody comprises a heavy chain comprising amino acids 1-445 of SEQ ID NO:40 or the amino acid sequence of SEQ ID NO:40, a light chain comprising the amino acid sequence of SEQ ID NO:26, a heavy chain comprising amino acids 1-444 of SEQ ID NO:45 or the amino acid sequence of SEQ ID NO:45 and a light chain comprising the amino acid sequence of SEQ ID NO:31.

90. The bispecific antibody of any one of claims 1, 2, 10-25, and 27-29, wherein the bispecific antibody comprises a heavy chain comprising amino acids 1-445 of SEQ ID NO:41 or the amino acid sequence of SEQ ID NO:41, a light chain comprising the amino acid sequence of SEQ ID NO:26, a heavy chain comprising amino acids 1-444 of SEQ ID NO:46 or the amino acid sequence of SEQ ID NO:46 and a light chain comprising the amino acid sequence of SEQ ID NO:31.

91. The bispecific antibody of any one of claims 1-90, wherein the bispecific antibody is capable of binding MerTK and PDL1 simultaneously.

92. The bispecific antibody of any one of claims 1-91, wherein the bispecific antibody reduces efferocytosis by a phagocytic cell.

93. The bispecific antibody of claim 92, wherein the bispecific antibody reduces efferocytosis with an IC50 value of about 4 nM to about 16 nM.

94. The bispecific antibody of claim 93, wherein the bispecific antibody reduces efferocytosis with an IC50 value of about 4 nM to about 5 nM, or about 15 nM to about 16 nM.

95. The bispecific antibody of claim 93 or 49, wherein the phagocytic cell is a macrophage, a tumor-associated macrophage, or a dendritic cell.

96. The bispecific antibody of claim 95, wherein the phagocytic cell is a macrophage.

97. The bispecific antibody of any one of claims 1-96, wherein the bispecific antibody inhibits tumor growth.

98. The bispecific antibody of any one of claims 1-97, wherein the bispecific antibody reduces binding of ProS to MerTK.

99. The bispecific antibody of any one of claims 1-98, wherein the bispecific antibody reduces binding of Gas6 to MerTK.

100. The bispecific antibody of any one of claims 1-99, wherein the bispecific antibody reduces binding of ProS to MerTK and reduces the binding of Gas6 to MerTK.

101. The bispecific antibody of any one of claims 1-100, wherein the bispecific antibody binds to human MerTK with a binding affinity of about 2 nM to about 30 nM, optionally wherein the bispecific antibody binds to human MerTK with a binding affinity of about 2 nM or about 30 nM.

102. The bispecific antibody of any one of claims 1-101, wherein the bispecific antibody also binds to cynomolgus monkey MerTk.

103. The bispecific antibody of claim 102, wherein the bispecific antibody binds to cynomolgus MerTK with a binding affinity of about 2 nM to about 30 nM, optionally wherein the bispecific antibody binds to cynomolgus MerTK with a binding affinity of about 2 nM or about 30 nM.

104. The bispecific antibody of any one of claims 1-103, wherein the bispecific antibody also binds to murine MerTK.

105. The bispecific antibody of claim 104, wherein the bispecific antibody binds to murine MerTK with a binding affinity of about 40 nM.

106. The bispecific antibody of any one of claims 1-103, wherein the bispecific antibody does not bind to murine MerTK.

107. The bispecific antibody of any one of claims 1-106, wherein the bispecific antibody reduces Gas6-mediated phosphorylation of AKT.

108. The bispecific antibody of any one of claims 1-107, wherein the bispecific antibody reduces Gas6-mediated phosphorylation of AKT with an IC50 value of about 9 nM to about 13 nM, optionally wherein the bispecific antibody reduces Gas6-mediated phosphorylation of AKT with an IC50 value of about 9 nM or about 13 nM.

109. The bispecific antibody of any one of claims 1-108, wherein the bispecific antibody is a murine antibody, a human antibody, a humanized antibody, a monoclonal antibody, a multivalent antibody, a conjugated antibody, or a chimeric antibody.

110. A humanized form of the bispecific antibody of any one of claims 1-109.

111. The bispecific antibody of any one of claims 1-110, wherein the bispecific antibody is a recombinant antibody.

112. The bispecific antibody of any one of claims 1-111, wherein the bispecific antibody is an isolated antibody.

113. An isolated nucleic acid comprising a nucleic acid sequence encoding the bispecific antibody of any one of claims 1-112.

114. A vector comprising the nucleic acid of claim 113.

115. An isolated host cell comprising the nucleic acid of claim 113 or the vector of claim 114.

116. An isolated host cell comprising (i) a nucleic acid comprising a nucleic acid sequence encoding the variable heavy chain of the first antigen binding domain of the bispecific antibody of any one of claims 1-112; (ii) a nucleic acid comprising a nucleic acid sequence encoding the variable light chain of the first antigen binding domain of the bispecific antibody; (iii) a nucleic acid comprising a nucleic acid sequence encoding the variable heavy chain of the second antigen binding domain of the bispecific antibody; and (iv) a nucleic acid comprising a nucleic acid sequence encoding the variable light chain of the second antigen binding domain of the bispecific antibody;

117. An isolated host cell comprising (i) a nucleic acid comprising a nucleic acid sequence encoding the heavy chain comprising the variable heavy chain the first antigen binding domain of the bispecific antibody of any one of claims 1-112; (ii) a nucleic acid comprising a nucleic acid sequence encoding a light chain comprising the variable light chain of the first antigen binding domain of the bispecific antibody; (iii) a nucleic acid comprising a nucleic acid sequence encoding a heavy chain comprising the variable heavy chain of the second antigen binding domain of the bispecific antibody; and (iv) a nucleic acid comprising a nucleic acid sequence encoding a light chain comprising the variable light chain of the second antigen binding domain of the bispecific antibody.

118. A method of producing a bispecific antibody that binds to human MerTK and PDL1, the method comprising culturing the cell of any one of claims 115-117 so that the bispecific antibody is produced.

119. The method of claim 118, further comprising recovering the bispecific antibody produced by the cell.

120. A bispecific antibody produced by the method of claim 118 or 119.

121. A pharmaceutical composition comprising the bispecific antibody of any one of claim 1-112 or 120 and a pharmaceutically acceptable carrier.

122. A method of treating cancer in an individual, the method comprising administering to an individual a therapeutically effective amount of the bispecific antibody of any one of claim 1-112 or 120 or the pharmaceutical composition of claim 121.

123. The method of claim 122, wherein the cancer is colon cancer, ovarian cancer, liver cancer, or endometrial cancer.

124. The method of claim 122 or 123, wherein the administration does not lead to a retinal pathology in the individual.

125. A method for detecting MerTK in a sample comprising contacting said sample with the bispecific antibody of any one of claim 1-112 or 120.
